(19) United States
(12) Patent Application Publication
(10) Pub. No.: US 2020/0038418 A1
(43) Pub. Date: Feb. 6, 2020

(54) METHODS OF TREATING AUTISM SPECTRUM DISORDER USING AMINOSTEROL COMPOSITIONS

(71) Applicant: Enterin, Inc., Philadelphia, PA (US)

(72) Inventors: Denise Barbut, Philadelphia, PA (US); Michael Zasloff, Philadelphia, PA (US)

(73) Assignee: Enterin, Inc., Philadelphia, PA (US)

(21) Appl. No.: 16/530,200

(22) Filed: Aug. 2, 2019

Related U.S. Application Data
(60) Provisional application No. 62/714,468, filed on Aug. 3, 2018.
(60) Provisional application No. 62/714,470, filed on Aug. 3, 2018.
(60) Provisional application No. 62/789,439, filed on Jan. 7, 2019.

(51) Int. Cl. A61K 31/575; A61K 9/00; A61P 25/28

(52) U.S. Cl. CPC A61K 31/575; A61K 9/0043; A61K 9/0053; A61K 45/06; A61K 9/0019; A61P 25/28; A61K 9/0075

(57) ABSTRACT

The present application relates generally to compositions and methods for treating, preventing, and/or slowing the onset or progression of autism spectrum disorder (ASD) and a variety of symptoms related thereto with aminosterols or pharmaceutically acceptable salts or derivatives thereof.

### CROSS-REFERENCE TO RELATED APPLICATIONS

This application claims the priority benefits under 35 USC § 119 to U.S. provisional Application No. 62/714,470, filed Aug. 3, 2018; U.S. provisional Application No. 62/714,468, filed Aug. 3, 2018; and U.S. provisional Application No. 62/789,439, filed Jan. 7, 2019, the entire contents of which are incorporated herein by reference in their entirety

### FIELD

The present application relates generally to methods for treating, preventing, and/or slowing the onset or progression of autism spectrum disorder (ASD) and/or a variety of symptoms and disorders related thereto with aminosterols or pharmaceutically acceptable salts or derivatives thereof.

### BACKGROUND

Amino sterols are amino derivatives of a sterol. Examples of aminosterols include squalamine and Aminosterol 1436 (also known as trodusquemine and MSI-1436).

Squalamine is a unique compound with a structure of a bile acid coupled to a polyamine (spermidine):

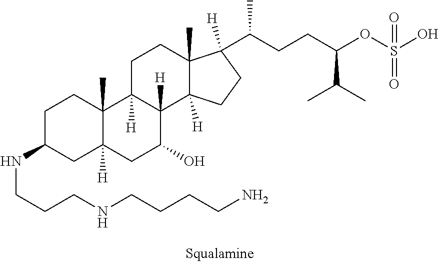

The discovery of squalamine, the structure of which is shown above, was reported by Michael Zasloff in 1993 (U.S. Pat. No. 5,192,756). Squalamine was discovered in various tissues of the dogfish shark (Squalus acanthias) in a search for antibacterial agents. The most abundant source of squalamine is in the livers of Squalus acanthias, although it is found in other sources, such as lampreys (Yun et al., 2007).

Several clinical trials have been conducted relating to the use of squalamine, including the following:

(1) ClinicalTrials.gov Identifier NCT01769183 for “Squalamine for the Treatment in Proliferative Diabetic Retinopathy,” by Elman Retina Group (6 participants; study completed August 2014);

(2) ClinicalTrials.gov Identifier NCT02727881 for “Efficacy and Safety Study of Squalamine Ophthalmic Solution in Subjects With Neovascular AMD (MAKO),” by Ohr Pharmaceutical Inc. (230 participants; study completed December 2017);

(3) ClinicalTrials.gov Identifier NCT02614937 for “Study of Squalamine Lactate for the Treatment of Macular Edema Related to Retinal Vein Occlusion,” by Ohr Pharmaceutical Inc. (20 participants; study completed December 2014);

(4) ClinicalTrials.gov Identifier NCT01678963 for “Efficacy and Safety of Squalamine Lactate Eye Drops in Subjects With Neovascular (Wet) Age-related Macular Degeneration (AMD),” by Ohr Pharmaceutical Inc. (142 participants; study completed March 2015);

(5) ClinicalTrials.gov Identifier NCT00333476 for “A Study of MSI-1256F (Squalamine Lactate) To Treat “Wet” Age-Related Macular Degeneration,” by Genaera Corporation (140 participants; study terminated);

(6) ClinicalTrials.gov Identifier NCT00094120 for “MSI-1256F (Squalamine Lactate) in Combination With Verteporfin in Patients With “Wet” Age-Related Macular Degeneration (AMD),” by Genaera Corporation (60 participants; study completed February 2007);

(7) ClinicalTrials.gov Identifier NCT00089830 for “A Safety and Efficacy Study of MSI-1256F (Squalamine Lactate) To Treat “Wet” Age-Related Macular Degeneration,” by Genaera Corporation (120 participants; study completed May 2007); and

(8) ClinicalTrials.gov Identifier NCT03047629 for Evaluation of Safety and Tolerability of ENT-01 for the Treatment of Parkinson's Disease Related Constipation (RASMET) (50 participants; study completed Jun. 14, 2018).

Aminosterol 1436 is an aminosterol isolated from the dogfish shark, which is structurally related to squalamine (U.S. Pat. No. 5,840,936). It is also known as MSI-1436, trodusquemine and produlestan.

Several clinical trials have been conducted relating to the use of Aminosterol 1436:

(1) ClinicalTrials.gov Identifier NCT00509132 for “A Phase I, Double-Blind, Randomized, Placebo-Controlled Ascending IV Single-Dose Tolerance and Pharmacokinetic Study of Trodusquemine in Healthy Volunteers,” by Genaera Corp.;

(2) ClinicalTrials.gov Identifier NCT00606112 for “A Single Dose, Tolerance and Pharmacokinetic Study in Obese or Overweight Type 2 Diabetic Volunteer,” by Genaera Corp.;

(3) ClinicalTrials.gov Identifier NCT00806338 for “An Ascending Multi-Dose, Tolerance and Pharmacokinetic Study in Obese or Overweight Type 2 Diabetic Volunteers,” by Genaera Corp.; and

(4) ClinicalTrials.gov Identifier: NCT02524951 for “Safety and Tolerability of MSI-1436C in Metastatic Breast Cancer,” by DepyMed Inc.

ASD is a range of developmental disorders characterized by trouble with social interaction and communication and by restricted and repetitive behavior. ASD may include impairments regarding joint attention and social reciprocity, challenges with verbal language cues, and poor nonverbal communication skills. Symptoms of ASD generally lead to problems with friendships, romantic relationships, daily living, and vocational success.

Specific causes of ASD are yet to be found. Risk factors have been identified. Of all of the conjectured causes of ASD, genetics have proven to give the highest risk of being diagnosed with ASD. If a family member is on the autism spectrum, there is a 50% chance that another member of the family will be diagnosed with the disorder as well, and being a twin gives a 69 to 90% chance of an ASD diagnosis (Frith et al., 2005).

The U.S. Center for Disease Control's most recent estimate is that 1 out of every 68 children, or 14.7 per 1,000, have some form of ASD as of 2010 (CDC.gov, 2018). Reviews tend to estimate a prevalence of 6 per 1,000 for autism spectrum disorders as a whole (Newschaffer et al., 2007), although prevalence rates vary for each of the developmental disorders in the spectrum. Autism prevalence has been estimated at 1-2 per 1,000, Asperger syndrome at roughly 0.6 per 1,000, childhood disintegrative disorder at 0.02 per 1,000, and PDD-NOS at 3.7 per 1,000 (Newschaffer et al., 2007). These rates are consistent across cultures and ethnic groups, as autism is considered a universal disorder.

The full potential of aminosterols for use in treatment has yet to be determined.

### SUMMARY

The present application relates generally to methods for treating, preventing and/or slowing progression of ASD and/or a related symptom with aminosterols or pharmaceutically acceptable salts or derivatives thereof.

In one embodiment, encompassed are methods of treating, preventing, and/or slowing the onset or progression of autism spectrum disorder (ASD) and/or a related symptom in a subject in need comprising administering to the subject a therapeutically effective amount of at least one aminosterol or a salt or derivative thereof, provided that the administering does not comprise oral administration. In exemplary embodiments, the method of administration comprises nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In another embodiment, the method of administration comprises nasal administration.

The amino sterol or a salt or derivative thereof can be formulated with one or more pharmaceutically acceptable carriers or excipients. Preferably the aminosterol is a pharmaceutically acceptable grade of the amino sterol.

The therapeutically effect amount of the at least one aminosterol or a salt or derivative thereof in the methods of the invention can be, for example, about 0.1 to about 20 mg/kg, about 0.1 to about 15 mg/kg, about 0.1 to about 10 mg/kg, about 0.1 to about 5 mg/kg, or about 0.1 to about 2.5 mg/kg body weight of the subject. In another aspect, the therapeutically effect amount of the at least one aminosterol or a salt or derivative thereof in the methods of the invention can be, for example, about 0.001 to about 500 mg/day, about 0.001 to about 250 mg/day, about 0.001 to about 125 mg/day, about 0.001 to about 50 mg/day, about 0.001 to about 25 mg/day, or about 0.001 to about 10 mg/day.

Certain embodiments describe the determination and administration of a “fixed aminosterol dose” that is not age, size, or weight dependent but rather is individually calibrated. Thus, in another embodiment, encompassed is a method of treating, preventing and/or slowing progression ASD and/or a related symptom in a subject in need comprising (a) determining a dose of an aminosterol or a salt or derivative thereof for the subject, wherein the amino sterol dose is determined based on the effectiveness of the aminosterol dose in improving or resolving an ASD symptom being evaluated, (b) followed by administering the amino sterol dose to the subject for a period of time, wherein the method comprises (i) identifying an ASD symptom to be evaluated; (ii) identifying a starting dose of an aminosterol or a salt or derivative thereof for the subject; and (iii) administering an escalating dose of the aminosterol or a salt or derivative thereof to the subject over a period of time until an effective dose for the ASD symptom being evaluated is identified, wherein the effective dose is the aminosterol dose where improvement or resolution of the ASD symptom is observed, and fixing the dose of the aminosterol or a salt or derivative thereof at that level for that particular ASD symptom in that particular subject. In this aspect of the invention, the aminosterol or a salt or derivative thereof can be administered via any pharmaceutically acceptable means. For example, the aminosterol or a salt or derivative thereof can be administered via any pharmaceutically acceptable means, such as orally, intranasally, by injection (IV, IP, or IM) or any combination thereof. The aminosterol or a salt or derivative thereof can be formulated with one or more pharmaceutically acceptable carriers or excipients.

In one embodiment, starting dosages of the aminosterol or a salt or derivative thereof for oral administration can range, for example, from about 1 mg up to about 175 mg/day, or any amount in-between these two values. In another embodiment, the composition is administered orally and the dosage of the amino sterol or a salt or derivative thereof is escalated in about 25 mg increments. In yet another embodiment, the composition is administered orally and the dose of the aminosterol or a salt or derivative thereof for the subject following dose escalation is fixed at a range of from about 1 mg up to about 500 mg/day, or any amount in-between these two values.

In another embodiment, the composition is administered intranasally (IN) and the starting aminosterol or a salt or derivative thereof dosage ranges from about 0.001 mg to about 3 mg/day, or any amount in-between these two values. For example, the starting aminosterol dosage for IN administration, prior to dose escalation, can be, for example, about 0.001, about 0.005, about 0.01, about 0.02, about 0.03, about 0.05, about 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 1.0, about 1.1, about 1.25, about 1.3, about 1.4, about 1.5, about 1.6, about 1.7, about 1.75, about 1.8, about 1.9, about 2.0, about 2.1, about 2.25, about 2.3, about 2.4, about 2.5, about 2.6, about 2.7, about 2.75, about 2.8, about 2.9, or about 3 mg/day.

In another embodiment, the composition is administered IN and the dosage of the aminosterol or a salt or derivative thereof is escalated in increments of about 0.01, about 0.05, about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

Finally, in yet another embodiment, the composition is administered IN and the dose of the aminosterol or a salt or derivative thereof for the subject following escalation is fixed at a range of from about 0.001 mg up to about 6 mg/day, or any amount in-between these two values. In yet a further embodiment, the amino sterol composition is administered intranasally and the dose of the aminosterol or a salt or derivative thereof for the subject following dose escalation is a dose which is sub therapeutic when given orally or by injection.

In one embodiment, the dosage of the amino sterol or a salt or derivative thereof is escalated every about 3 to about 5 days. In another embodiment, the dose of the aminosterol or a salt or derivative thereof is escalated about 1×/week, about 2×/week, about every other week, or about 1×/month.

In another embodiment, the fixed dose of the aminosterol or a salt or derivative thereof is given once per day, every other day, once per week, twice per week, three times per week, four times per week, five times per week, six times per week, every other week, or every few days. In addition, the fixed dose of the aminosterol or a salt or derivative thereof can be administered for a first period of time of administration, followed by a cessation of administration for a second period of time, followed by resuming administration upon recurrence of autism or a symptom of autism. For example, the fixed aminosterol dose can be incrementally reduced after the fixed dose of aminosterol or a salt or derivative thereof has been administered to the subject for a period of time. Alternatively, the fixed dose of the aminosterol or a salt or derivative thereof is varied plus or minus a defined amount to enable a modest reduction or increase in the fixed dose. For example, the fixed dose of the aminosterol or a salt or derivative thereof can be increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%.

In another embodiment, the starting amino sterol or a salt or derivative thereof dose is higher if the ASD symptom being evaluated is severe. In one embodiment, the fixed escalated dose of the aminosterol or a salt or derivative thereof reverses dysfunction caused by the ASD and treats, prevents, improves, and/or resolves the symptom being evaluated.

In yet another embodiment, the ASD symptom to be evaluated can be selected from the group consisting of (a) a symptom from the Autism Spectrum Rating Scales (ASRS™) selected from the group consisting of social skills, communication skills, unusual behavior, self-regulation ability, peer socialization, adult socialization, atypical language, and stereotypy; (b) a symptom from the Autism Diagnostic Observation Schedule (ADOS) selected from the group consisting of performance in Module 1 (used with children who use little or no phrase speech), performance in Module 2 (used with subjects that use phrase speech but who do not speak fluently), performance in Module 3 (used with younger subjects who are verbally fluent), and performance in Module 4 (used with adolescents and adults who are verbally fluent); (c) a symptom from the Autism Diagnostic Interview-Revised (ADI-R), wherein the symptom is selected from the group consisting of emotional sharing, offering and seeking comfort, social smiling, responding to other children, stereotyped utterances, pronoun reversal, social usage of language, preoccupation with unusual things, hand and finger mannerism, unusual sensory interests, self-injury, aggression, and overactivity; (d) failure to respond to name; (e) failure to point at objects of interest; (f) inability to role play; (g) avoidance of eye contact; (h) preference to be alone; (i) inability to understand feelings of others; (j) no speech or delayed development of speech; (k) echolalia and/or palilalia; (l) answering questions with unrelated answers; (m) upset by minor changes; (n) obsessive interests; (o) lining-up or stacking of objects; (p) repetitive motion; (q) avoidance of physical contact with others; (r) lack of awareness of danger; (s) sleep disorder or sleep disturbance; (r) constipation; (s) cognitive impairment; (t) gastrointestinal (GI) problems; (u) epilepsy; (v) feeding issues; (w) Attention-deficit/hyperactivity disorder (ADHD); (x) anxiety; (y) depression; (z) Obsessive compulsive disorder (OCD); (aa) schizophrenia; (bb) Bipolar Disorder; and (cc) neurodegeneration associated with ASD. Examples of GI issues/disorders include, but are not limited to, chronic constipation, abdominal pain, gastroesophageal reflux, and bowel inflammation.

In some embodiments, the ASD symptom is a sleep disorder or sleep disturbance and is selected from the group consisting of decreased quantity of REM sleep, increased undifferentiated sleep, immature organization of eye movements into discrete bursts during REM sleep, decreased time in bed, decreased total sleep time, decreased REM sleep latency, increased proportion of stage 1 sleep, circadian rhythm disruption, and any combination thereof.

In some embodiments, the symptom to be evaluated is a sleep disorder or sleep disturbance wherein (a) the method results in a positive change in the sleeping pattern of the subject; (b) the method results in a positive change in the sleeping pattern of the subject, wherein the positive change is defined as (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (c) as a result of the method the subject obtains the total number of hours of sleep recommended by a medical authority for the age group of the subject.

In some embodiments, the ASD symptom to be evaluated is a avoidance of eye contact, wherein (a) the method results in a positive change in the amount of eye contact engaged in by the subject; (b) the method results in a positive change in the amount of eye contact engaged in by the subject, wherein the positive change is defined as (i) an increase in the amount of eye contact of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of instances in which the subject avoids eye contact selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, the ASD symptom to be evaluated is echolalia (unsolicited repetition of vocalizations made by another person) and/or palilalia (repetition of vocalizations made by the same person), wherein (a) the method results in a decreased number or severity of instances in which the subject engages in echolalia and/or palilalia; (b) the method results in a decreased number or severity of instances in which the subject engages in echolalia and/or palilalia and the decrease in number or severity of instances in which the subject engages in echolalia and/or palilalia is defined as a reduction in engagement in echolalia and/or palilalia selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject ceasing to engage in echolalia and/or palilalia.

In some embodiments, the ASD symptom to be evaluated is self-injury wherein (a) the method results in a decreased number or severity of instances in which the subject engages in self-injury; (b) the method results in a decreased number or severity of instances in which the subject engages in self-injury and the decrease is selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject ceasing to engage in self-injury.

In some embodiments, the ASD symptom to be evaluated is repetitive motion wherein (a) the method results in a decreased number or severity of instances in which the subject engages in repetitive motion; (b) the method results in a decreased number or severity of instances in which the subject engages in repetitive motion and the decrease is selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject ceasing to engage in repetitive motion.

In some embodiments, the ASD symptom to be evaluated is constipation, wherein (a) the fixed escalated aminosterol dose causes the subject to have a bowel movement; (b) the method results in an increase in the frequency of bowel movement in the subject; (c) the method results in an increase in the frequency of bowel movement in the subject and the increase in the frequency of bowel movement is defined as (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (d) as a result of the method the subject has the frequency of bowel movement recommended by a medical authority for the age group of the subject; and/or (e) the starting amino sterol dose is determined by the severity of the constipation, wherein (i) if the average complete spontaneous bowel movement (CSBM) or spontaneous bowel movement (SBM) is one or less per week, then the starting aminosterol dose is at least about 150 mg/day; and (ii) if the average CSBM or SBM is greater than one per week, then the starting amino sterol dose is about 75 mg/day or less.

In some embodiments, the ASD symptom to be evaluated is cognitive impairment, and (a) progression or onset of the cognitive impairment is slowed, halted, or reversed over a defined period of time following administration of the fixed escalated dose of the aminosterol or a salt or derivative thereof, as measured by a medically-recognized technique; and/or (b) the cognitive impairment is positively impacted by the fixed escalated dose of the aminosterol or a salt or derivative thereof, as measured by a medically-recognized technique; (c) the cognitive impairment is positively impacted by the fixed escalated dose of the aminosterol or a salt or derivative thereof, as measured by a medically-recognized technique and the positive impact on and/or progression of cognitive impairment is measured quantitatively or qualitatively by one or more techniques selected from the group consisting of ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, and a computerized tested selected from Cantab Mobile, Cognigram, Cognivue, Cognision, or Automated Neuropsychological Assessment Metrics Cognitive Performance Test (CPT); and/or (d) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In embodiments where the ASD symptom to be evaluated is depression, (a) treating the depression may prevent and/or delay the onset and/or progression of ASD; (b) the method may result in improvement in a subject's depression, as measured by one or more clinically-recognized depression rating scale; (c) the method may result in improvement in a subject's depression, as measured by one or more clinically-recognized depression rating scale and the improvement can be in one or more depression characteristics selected from the group consisting of mood, behavior, bodily functions such as eating, sleeping, energy, and sexual activity, and/or episodes of sadness or apathy; and/or (d) the method may result in improvement in a subject's depression, as measured by one or more clinically-recognized depression rating scale, and the improvement a subject experiences following treatment can be about 5, about 10, about 15, about 20, about 25, about 30, about 35, about 40, about 45, about 50, about 55, about 60, about 65, about 70, about 75, about 80, about 85, about 90, about 95 or about 100%.

In embodiments where the ASD symptom to be evaluated is neurodegeneration correlated with ASD, (a) treating the neurodegeneration may prevent and/or delay the onset and/or progression of the ASD; (b) the method may result in treating, preventing, and/or delaying the progression and/or onset of neurodegeneration in the subject; (c) progression or onset of the neurodegeneration can be slowed, halted, or reversed over a defined period of time following administration of the fixed escalated dose of the aminosterol or a salt or derivative thereof, as measured by a medically-recognized technique; and/or (d) the neurodegeneration can be positively impacted by the fixed escalated dose of the aminosterol or a salt or derivative thereof, as measured by a medically-recognized technique. In further embodiments, (a) the positive impact and/or progression of neurodegeneration can be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or (b) the progression or onset of neurodegeneration can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

For all of the embodiments described herein, each defined period of time is independently selected from the group consisting of about 1 day to about 10 days, about 10 days to about 30 days, about 30 days to about 3 months, about 3 months to about 6 months, about 6 months to about 12 months, and about greater than 12 months.

In some embodiments, the amino sterol or a salt or derivative thereof is administered in combination with at least one additional active agent to achieve either an additive or synergistic effect. In some embodiments, the additional active agent is administered via a method selected from the group consisting of (a) concomitantly; (b) as an admixture; (c) separately and simultaneously or concurrently; and (d) separately and sequentially.

In some embodiments, the additional active agent is a different amino sterol or a salt or derivative thereof from that administered in the method described herein.

In some embodiments, the method comprises administering a first amino sterol or a salt or derivative thereof which is aminosterol 1436 or a salt or derivative thereof administered intranasally and a second amino sterol or a salt or derivative thereof which is squalamine or a salt or derivative thereof administered orally. In some embodiments, the additional active agent is an active agent used to treat ASD or a symptom thereof.

In some embodiments, the active agent is selected from the group consisting of serotonin-norepinephrine reuptake inhibitors such as venlafaxine, (Effexor®); selective serotonin reuptake inhibitor such as fluoxetine (Prozac®) or citalopram (Celexa®); N-methyl D-aspartate (NMDA) antagonists such as memantine (Namenda®); dopamine receptor antagonists such as haloperidol (Haldol®); a loop diuretic such as bumetanide; an acetylcholinesterase inhibitor such as rivastigmine (Exelon®); a central nervous system stimulant such as methylphenidate (Ritalin®) or amphetamine (Adderall®); and/or atypical antipsychotics such as risperidone (Risperdol®), aripiprazole (Abilify®), ziprasidone (Geodon®), paliperidone (Invega®), or clozapine (Clozaril®).

For all of the methods of the invention, in one embodiment each amino sterol dose is taken on an empty stomach, optionally within about two hours of the subject waking. In another embodiment for all of the methods of the invention, no food is taken or consumed after about 60 to about 90 minutes of taking the dose of the aminosterol or a salt or derivative thereof. Further, in yet another embodiment applicable to all of the methods of the invention, the aminosterol or a salt or derivative thereof can be a pharmaceutically acceptable grade of at least one aminosterol or a pharmaceutically acceptable salt or derivative thereof. For all of the methods of the invention the subject can be a human.

The amino sterol or a salt or derivative thereof utilized in the methods of the invention can be: (a) isolated from the liver of Squalus acanthias; (b) a synthetic aminosterol; (c) squalamine or a pharmaceutically acceptable salt thereof; (d) a squalamine isomer; (e) the phosphate salt of squalamine; (f) aminosterol 1436 or a pharmaceutically acceptable salt thereof; (g) an aminosterol 1436 isomer; (h) the phosphate salt of aminosterol 1436; (i) a compound comprising a sterol nucleus and a polyamine attached at any position on the sterol, such that the molecule exhibits a net charge of at least +1; (j) a compound comprising a bile acid nucleus and a polyamine, attached at any position on the bile acid, such that the molecule exhibits a net charge of at least +1; (k) a derivative modified to include one or more of the following: (i) substitutions of the sulfate by a sulfonate, phosphate, carboxylate, or other anionic moiety chosen to circumvent metabolic removal of the sulfate moiety and oxidation of the cholesterol side chain; (ii) replacement of a hydroxyl group by a non-metabolizable polar substituent, such as a fluorine atom, to prevent its metabolic oxidation or conjugation; and (iii) substitution of one or more ring hydrogen atoms to prevent oxidative or reductive metabolism of the steroid ring system; and/or (1) a derivative of squalamine or aminosterol 1436 modified through medical chemistry to improve bio-distribution, ease of administration, metabolic stability, or any combination thereof. In one embodiment, the aminosterol is selected from the group consisting aminosterol 1436 or a pharmaceutically acceptable salt thereof, squalamine or a pharmaceutically acceptable salt thereof, or a combination thereof. In another embodiment, the aminosterol is a phosphate salt.

Further, the aminosterol composition can comprise, for example, one or more of the following: an aqueous carrier, a buffer, a sugar, and/or a polyol compound.

Both the foregoing summary and the following description of the drawings and detailed description are exemplary and explanatory. They are intended to provide further details of the invention, but are not to be construed as limiting. Other objects, advantages, and novel features will be readily apparent to those skilled in the art from the following detailed description of the invention.

### BRIEF DESCRIPTION OF THE DRAWINGS

FIGS. 1A and 1B show prokinetic activity of squalamine (ENT-01, a synthetic squalamine salt comprising squalamine as the active ion). As shown in panel A, in Stage 1 (single dose), cumulative prokinetic response rate was defined as the proportion of patients who had a complete spontaneous bowel movements (CSBM) within 24 hours of dosing. In Stage 2 (daily dosing), a prokinetic response was defined as the fraction of patients who had a CSBM within 24 hours of dosing on at least 2 out of 3 days at any given dose. As shown in panel B, the prokinetic dose of squalamine was significantly related to baseline constipation severity (p=0.00055). Patients with baseline CSBM<1 required a higher dose (mean, 192 mg) of squalamine than patients with CSBM≥1 (mean, 120 mg).

FIG. 2 is a schematic (flowchart) showing patient disposition in Stage 2 of the clinical study. (1) Patients first enrolled (n=40); (2) 6 patients failed to meet dosing criteria and were excluded; (3) 34 patients were dosed; (4) 5 patients were discontinued; 3 patients withdrew consent (with 1 patient lost to follow up and 2 patients withdrew because of diarrhea); and 2 patients discontinued because of an adverse event (recurrent dizziness after medication); (5) 31 patients had an assessable prokinetic response; and (6) 29 patients completed dosing.

FIG. 3 is a chart of total sleep time in relation to squalamine dose. Total sleep time was obtained from the sleep diary by subtracting awake time during the night from total time spent in bed. Total sleep time per night was logged for each patient at baseline, each dosing period and at washout, and the means were determined. The light grey bar represents the baseline value for each cohort at a given dose level and the dark grey bar represents the value for the same cohort at the stated dose of squalamine (ENT-01; Kenterin™). The number of patients represented at each value are: Baseline, 33; 75 mg, 21; 100 mg, 28; 125 mg, 18; 150 mg, 15; 175 mg, 12; 200 mg, 7; 225 mg, 3; 250 mg, 2; washout, 33. P values were as follows: 75 mg, p=0.4; 100 mg, p=0.1; 125 mg, p=0.3; 150 mg, p=0.07; 175 mg, p=0.03; 200 mg, p=0.3; 225 mg, p=0.5; 250 mg, p=0.3; wash-out, p=0.04 (paired t test).

FIG. 4 shows the effect of squalamine (ENT-01) on circadian rhythm. The figure depicts the mean waveform of temperature under three conditions per patient: baseline (Line #1), treatment with highest drug dose (Line #2), and washout (Line #3). Each mean waveform is double plotted for better visualization. Low temperatures indicate higher activation, while higher values are associated with drowsiness and sleepiness. The top black bar indicates a standard rest period from 23:00 to 07:00 h.

FIGS. 5A-F show the effect of squalamine (ENT-01) on circadian rhythm. The figures depict the results of circadian non-parametric analysis of wrist skin temperature rhythm throughout each condition (baseline, treatment with highest dose of squalamine (ENT-01) and washout). The following parameters were measured: Inter-daily variability (FIG. 5A), inter-daily stability (IS) (FIG. 5B), relative amplitude (RA) (FIG. 5C), circadian function index (FIG. 5D), M5V (FIG. 5E), which refers to the five consecutive hours with the highest temperature or high somnolence, and L10V (FIG. 5F), which indicates the mean of the ten consecutive hours with lowest temperature or high activation. The circadian function index (CFI) is an integrated score that ranges from 0 (absence of circadian rhythm) to 1 (robust circadian rhythm). Student's paired t-test, *p<0.05, **p<01, ***p<0.001. Values expressed as mean±SEM (n=12 in each condition).

### DETAILED DESCRIPTION

### I. Overview

The present application relates generally to methods for treating, preventing, and/or slowing the onset or progression of ASD and symptoms related thereto. The methods comprise administering one or more aminosterols or pharmaceutically acceptable salts or derivatives thereof to a subject in need. Certain embodiments describe the determination and administration of a “fixed dose” of an amino sterol or a pharmaceutically acceptable salt or derivative thereof that is not age, size, or weight dependent but rather is individually calibrated.

The amino sterol or a salt or derivative thereof can be formulated with one or more pharmaceutically acceptable carriers or excipients. Preferably the aminosterol is a pharmaceutically acceptable grade of the amino sterol.

In one embodiment, the invention encompasses a method of treating, preventing and/or slowing the onset or progression of schizophrenia and/or a related symptom in a subject in need comprising administering to the subject a therapeutically effective amount of at least one aminosterol or a salt or derivative thereof via any non-oral pharmaceutically acceptable method. Examples of such non-oral administration methods include but are not limited to nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof. In another aspect, the at least one amino sterol or a salt or derivative thereof is administered nasally.

In another embodiment, the present invention is directed to methods of treating, preventing, and/or slowing the onset or progression of ASD and/or a related symptom in a subject in need, comprising (a) determining a dose of an aminosterol or a salt or derivative thereof for the subject, wherein the amino sterol dose is determined based on the effectiveness of the aminosterol dose in improving or resolving an ASD symptom being evaluated; (b) followed by administering the dose of the amino sterol or a salt or derivative thereof to the subject for a period of time. The method of determining the aminosterol dose comprises (i) identifying an ASD symptom to be evaluated; (ii) identifying a starting dose of an aminosterol or a salt or derivative thereof for the subject; and (iii) administering an escalating dose of the amino sterol or a salt or derivative thereof to the subject over a period of time until an effective dose for the ASD symptom being evaluated is identified, wherein the effective dose is the dose of the amino sterol or a salt or derivative thereof where improvement or resolution of the ASD symptom is observed, and fixing the dose of the aminosterol or a salt or derivative thereof at that level for that particular ASD symptom in that particular subject.

A. Background Regarding Autism Spectrum Disorder

ASD is a range of neurodevelopmental disorders that includes autism and related conditions. Individuals diagnosed with ASD may present symptoms including problems in social communication and social interaction, and restricted, repetitive patterns of behavior, interests or activities. Symptoms are typically recognized between one and two years of age. Long term issues may include difficulties in creating and keeping relationships, maintaining a job, and performing daily tasks. The Diagnostic and Statistical Manual of Mental Disorders, 5th Edition (DSM-5) redefined the autism spectrum disorders to encompass the previous diagnoses of autism, Asperger syndrome, pervasive developmental disorder not otherwise specified (PDD-NOS), and childhood disintegrative disorder. Autism is the fastest growing developmental disorder. The prevalence of ASD in children increased by 119.4% from 2000 to 2010.

Asperger syndrome is an ASD characterized by significant difficulties in social interaction and nonverbal communication, along with restricted and repetitive patterns of behavior and interests. As a milder autism spectrum disorder (ASD), it differs from other ASDs by relatively normal language and intelligence. Physical clumsiness and unusual use of language are common. Signs usually begin before two years old and typically last for a subject's entire life.

Pervasive developmental disorder not otherwise specified (PDD-NOS) is an ASD also known as “atypical autism.” PDD-NOS may present late age of onset, atypical symptomatology, subthreshold symptomatology, or all of the preceding. Subjects with PDD-NOS may display communication difficulties (e.g., using and understanding language), difficulty with social behavior, difficulty with changes in routines or environments, uneven skill development (strengths in some areas and delays in others), unusual play with toys and other objects, repetitive body movements or behavior patterns, and preoccupation with fantasy, such as imaginary friends in childhood.

Childhood disintegrative disorder may be characterized by late onset of developmental delays, or severe and sudden reversals in language, social function, and motor skills.

The U.S. Center for Disease Control's most recent estimate is that 1 out of every 68 children, or 14.7 per 1,000, have some form of ASD as of 2010 (CDC.gov, 2018). Reviews tend to estimate a prevalence of 6 per 1,000 for autism spectrum disorders as a whole (Newschaffer et al., 2007), although prevalence rates vary for each of the developmental disorders in the spectrum. Autism prevalence has been estimated at 1-2 per 1,000, Asperger syndrome at roughly 0.6 per 1,000, childhood disintegrative disorder at 0.02 per 1,000, and PDD-NOS at 3.7 per 1,000 (Newschaffer et al., 2007). These rates are consistent across cultures and ethnic groups, as autism is considered a universal disorder (Mash and Barkley, 2003). Autism's economic costs for 2015 in the United States was estimated at $268 billion and projected to rise to $461 billion by 2025 (Leigh et al., 2015).

The symptoms of ASD are primarily marked by abnormal social interaction, for example, lack of empathy, failure to develop friendships, lack of social or emotional reciprocity, and impaired non-verbal behaviors including eye contact, facial expression, posture and gesture; restricted behaviors or interests, for example, abnormally intense focus on routine or repetition, inflexible routine, repetitive movements, reoccupation with parts of objects, arranging items into lines or patterns, and extreme interest in a narrow topic; unusual usage of language, for example, verbosity, abrupt transitions, literal interpretation, misconception of nuance, and oddities in loudness pitch, intonation and rhythm of speech; and absence of speech.

A reliable diagnosis for ASD can usually be made by the age of two years. Individuals with an ASD may present symptoms at various times of development (e.g., toddler, child, or adolescent), and symptom expression may vary over the course of development. After a child shows initial evidence of ASD tendencies, psychologists administer various psychological assessment tools to assess for ASD. Among these measurements, the Autism Diagnostic Interview-Revised (ADI-R) and the Autism Diagnostic Observation Schedule (ADOS) are often used.

Relationship Between ASD and PD:

Recent research has suggested a correlation between ASD and Parkinson's Disease. Specifically, researchers from North Carolina, USA, and Perth, Australia, noticed a trend of high rates of parkinsonism in adults with autism. Starkstein et al., 2015. The researchers report the findings of two studies. In the first study, 19 men with autism were included (with an average age of 57 years). When the researchers investigated the cardinal features of PD, they found that 22% of the subjects exhibited bradykinesia (or slowness of movement), 16% had a resting tremor, 32% displayed rigidity, and 15% had postural instability issues. In fact, 16% of the subjects actually met the criteria for a full diagnosis of PD (one of who was already responding well to L-dopa treatment). The second study was larger and involved 32 men and 5 women (with an average age of 51 years). 46% of the subjects in this study exhibited bradykinesia, 19% had a resting tremor, 19% displayed rigidity, and 19% had postural instability problems. In the second study, 32% of the subjects met the full diagnostic criteria for PD. Given this collective result, the researchers concluded that there may be an increased frequency of Parkinsonism in aged people with autism.

There are some interesting similarities between autism and PD. For example, there are genetic variations shared by both PD and autism. Approximately 10-20% of people with PD have a genetic variation in one of the PARK genes. In addition, one of the genes associated with autism is the Parkinson's associated gene, PARK2. Glessner et al. 2009.

Further, recent studies have evaluated the presence of alpha synuclein (αS) and tau protein in ASD subjects. Kadak et al. 2015. αS and tau proteins are thought to be related with the synaptic loss and cell death underlying several important neurodegenerative diseases. Serum levels of αS and tau were measured, and ASD severity was assessed at admission using the Childhood Autism Rating Scale (CARS) total score. The mean CARS score of the autism group on admission was 47.91 points (SD: 5.97). The results indicated that the mean serum αS and serum tau levels were significantly lower in children with ASD as compared with normal cases. There was a significant positive correlation between serum αS levels and serum levels of tau. Synaptic abnormality in autism may result from microglial activity. Furthermore, αS and tau aggregation may lead to synaptic dysfunction, and this may contribute to either neuronal or synaptic dysfunction or neurodegeneration. The study authors concluded that low levels of serum αS and tau may be implicated in the relationship between synaptic activity and autism.

In 2003, Braak proposed that PD begins with the formation of toxic αS aggregates within the ENS and manifests clinically as constipation in a majority of people years before the onset of motor symptoms. It was recently reported that αS is induced in the ENS in response to viral, bacterial and fungal infections and that excessive intraneuronal accumulation of αS promotes formation of toxic aggregates. As a result of the normal trafficking of αS aggregates from the ENS to the central nervous system (CNS) via afferent nerves such as the vagus, neurotoxic aggregates accumulate progressively within the brainstem and more rostral structures. Thus, inhibiting αS aggregation in the ENS may reduce the continuing disease process in conditions such as ASD and PD in both the ENS and CNS.

A strategy that targets neurotoxic aggregates of αS in the gastrointestinal tract represents a novel approach to the treatment of PD that may restore the function of enteric nerve cells and prevent retrograde trafficking to the brain. Such actions may potentially slow progression of PD in addition to restoring gastrointestinal function. It is theorized that given the commonalities observed between ASD and PD, the same treatment approach will be effective in treating, preventing, and/or slowing the onset of ASD. Accordingly but not to be bound by theory, the methods described herein are expected to apply to the treatment of any of the described symptoms as well as treatment, prevention, and/or slowing the onset of ASD and/or related symptoms.

Even more recently, another study evaluated the plasma αS and β-synuclein levels in 39 male children with ASD as compared to sex- and age-matched controls. W. Sriwimol and P. Limprasert, 2018. The study reported significant differences between plasma αS and β-synuclein levels between the male ASD subjects and control groups, with plasma αS level significantly lower and plasma β-synuclein significantly higher in ASD subjects. This result is interesting, given that other research has reported that αS plays an important role in synaptic functions, contributing to normal neuronal network activity and connectivity of the healthy brain. Additionally, other studies have suggested that dopamine homeostasis and neurotransmitter release may be controlled by αS level. Cheng et al. 2011.

The correlation between PD and ASD is also demonstrated by the commonality of symptoms. For example, many children began their descent into the symptoms of autism with gastrointestinal (GI) issues, usually constipation. Similarly, in recent years an important PD research focus has been on gut-related pathology, pathophysiology, and symptoms. Gastrointestinal dysfunction, in particular constipation, affects up to 80% of PD patients and idiopathic constipation is one of the strongest risk-factors for PD.

It has also been reported that constipation occurs in 85% of children with both autism and GI disorder and was most likely to occur in children who were younger, nonverbal and/or had significant social difficulties. Further, researchers have found a six-fold increase in communication disturbances in the group of children who had both ASD and GI disorder, as compared to children with ASD only. http://www.ageofautism.com/2015/10/parkinsons-autism-and-back-to-the-future.html, downloaded on Sep. 24, 2018.

In another embodiment, administration of a therapeutically effective fixed dose of a composition comprising at least one aminosterol or a salt or derivative thereof to an ASD subject results in improvement of one or more symptoms of ASD on one or more clinically accepted scoring metrics, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

Progression of neurodevelopmental pathology associated with ASD can be measured using well known techniques. For example, ADOS, ADI-R, The Childhood Autism Rating Scale, Autism Treatment Evaluation Checklist, and tests of cognitive functioning (e.g., The Peabody Picture Vocabulary Test).

A variety of neuroimaging techniques may be useful for the early diagnosis and/or measurement of progression of ASD. Examples of such techniques include but are not limited to neuroimaging, functional MRI (fMRI), magnetoencephalography (MEG), and functional connectivity magnetic resonance imaging (fcMRI) (Emerson et al., 2017). Combinations of these techniques can also be used to measure ASD disease progression.

For example, functional brain connections may be defined in 6-month old infants that correlate with 24-month old ASD subject's scores on measures of social behavior, language, motor development, and repetitive behavior, which are all features common to the diagnosis of ASD. A machine learning algorithm applied at age 6 months has a positive predictive value for the development of ASD in the subject. Id.

In one embodiment of the invention, the progression or onset of ASD is slowed or prevented over a defined period of time, following administration of a fixed dose of an aminosterol or a salt or derivative thereof according to the invention to a subject in need, as measured by a medically-recognized technique. For example, the progression or onset of ASD can be slowed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The medically recognized technique may be one or more of Autism Spectrum Rating Scales (ASRS™), Autism Diagnostic Observation Schedule (ADOS), and Autism Diagnostic Interview-Revised (ADI-R).

The period of time over which the progression or onset of ASD is measured can be for example, one or more months or one or more years, e.g., about 6 months, about 1 year, about 18 months, about 2 years, about 36 months, about 3, about 4, about 5, about 6, about 7, about 8, about 9, about 10, about 11, about 12, about 13, about 14, about 15, about 16, about 17, about 18, about 19, or about 20 years, or any amount of months or years in between the values of about 6 months to about 20 years or more.

In another embodiment of the invention, ASD may be positively impacted by administration of a fixed amino sterol dose according to the invention. A “positive impact” includes for example slowing advancement of the condition, improving one or more symptoms, etc.

B. Background Regarding the Experimental Data

As described in Example 1, a study was conducted in patients with Parkinson's disease (PD). PD is a progressive neurodegenerative disorder caused by shaking, rigidity, slowness of movement, and cognitive and behavioral problems. Studies have shown high rates of PD in autistic individuals, suggesting a link between the two conditions (Starkstein et al., 2015).

While the study described in Example 1 assessed patients with PD, many symptoms assessed and contemplated to be resolved by aminosterol treatment are not restored by the replacement of dopamine and are thus not unique to PD but also correlated with ASD. Examples of such symptoms include, but are not limited to, constipation, cognitive impairment, and sleep disturbances and disorders. Other relevant symptoms are described herein. All of these symptoms result from impaired function of neural pathways not restored by replacement of dopamine in PD subjects.

A strategy that targets neurotoxic aggregates of α-synuclein protein (αS) in the gastrointestinal tract represents a novel approach to the treatment of PD and potentially other neurodiseases including ASD. Regulation of αS levels is suggested to play a role in ASD pathogenesis (Sriwimol et al., 2018). Treatment and conditions described herein that may restore the function of enteric nerve cells and prevent retrograde trafficking to the brain. Such actions may potentially slow progression of the disease in addition to restoring gastrointestinal function.

Not to be bound by theory, it is believed that aminosterols target neurotoxic aggregates of αS in the gastrointestinal tract, and restore function of the enteric nerve cells. The now-functional enteric nerve cells prevent retrograde trafficking of proteins, such as alpha-synuclein, to the brain. In addition to restoring gastrointestinal function, this effect is believed to slow and possibly reverse ASD progression.

Constipation serves as symptom of many neurodiseases such as ASD. Not to be bound by theory, based on the data described herein, it is believed that aminosterols improve bowel function by acting locally on the gastrointestinal tract (as supported by the oral bioavailability <0.3%). An orally administered amino sterol such as squalamine, the active ion of ENT-01, stimulates gastro-intestinal motility in mice with constipation due to overexpression of human αS (West et al, manuscript in preparation). Perfusion of an aminosterol such as squalamine through the lumen of an isolated segment of bowel from the PD mouse model results in excitation of IPANs (intrinsic primary afferent neuron), the major sensory neurons of the ENS that communicate with the myenteric plexus, increasing the frequency of propulsive peristaltic contractions and augmenting neural signals projecting to the afferent arm of the vagus.

Systemic absorption of the aminosterol following oral administration was negligible both in this study and in prior studies involving mice, rats and dogs. Prior studies demonstrated that intravenous administration of squalamine was not associated with increased gastrointestinal motility, despite reaching systemic blood levels one thousand-fold greater than that achieved by orally administered squalamine. These data suggest that the effect is mediated by local action in the GI tract. The topical action would also explain why adverse events were largely confined to the gastrointestinal tract.

Several exploratory endpoints were incorporated into the trial described in Example 1 to evaluate the impact of an aminosterol on neurologic symptoms associated with a neurodisease such as PD. Following amino sterol treatment, the Unified Parkinson's Disease Rating Scale (UPDRS) score, a global assessment of motor and non-motor symptoms, showed significant improvement. Improvement was also seen in the motor component. The improvement in the motor component is unlikely to be due to improved gastric motility and increased absorption of dopaminergic medications, since improvement persisted during the 2-week wash-out period, i.e., in the absence of study drug (Table 12).

Improvements were also seen in cognitive function (MMSE scores) and sleep. Interestingly, most indices related to bowel function returned to baseline value by the end of the 2-week wash-out period while improvement in the CNS symptoms persisted. The rapid improvement in certain CNS symptoms is consistent with a mechanism whereby nerve impulses initiated from the ENS following amino sterol administration augment afferent neural signaling to the CNS. This may stimulate the clearance of αS aggregates within the afferent neurons themselves as well as the secondary and tertiary neurons projecting rostrally within the CNS, since it is known that neural stimulation is accompanied by increased neuronal autophagic activity (Shehata et al. 2012). It is believed that after cessation of amino sterol administration, the neurons of the CNS gradually re-accumulate an αS burden either locally or via trafficking from αS re-aggregation within the gut.

Disturbance of the circadian rhythm has been described in neurodiseases such as ASD and PD both clinically and in animal models and might play a role in the abnormal sleep architecture, dementia, mood and autonomic dysfunction associated with neurodiseases such as PD (Breen et al., 2014; Videnovic et al., 2017; Antonio-Rubio et al. 2015; Madrid-Navarro et al. 2018). Circadian rhythm was monitored through the use of a temperature sensor that continuously captured wrist skin temperature (Sarabia et al., 2008), an objective measure of the autonomic regulation of vascular perfusion (Videnovic et al., 2017). Circadian cycles of wrist skin temperature have been shown to correlate with sleep wake cycles, reflecting the impact of nocturnal heat dissipation from the skin on the decrease in core temperature and the onset of sleep (Sarabia et al. 2008; Ortiz-Tuleda et al., 2014). Oral administration of ENT-01 had a significant positive impact on the circadian rhythm of skin temperature in the 12 patients with evaluable data. Not to be bound by theory, it is believed that aminosterols could be affecting neuronal circuits involving the master clock (the suprachiasmatic nucleus) and its autonomic projections and opens the possibility of therapeutic correction of circadian dysfunction.

As described in Example 1, aminosterol dosing is patient specific, as the dose is likely related to the extent of neuronal damage, with greater neuronal damage correlating with the need for a higher aminosterol dose to obtain a desired therapeutic result. As described in greater detail herein, aminosterol dosing can range from about 0.01 to about 500 mg/day, with dosage determination described in more detail below.

### II. Methods of Treatment

The present application provides methods for the treatment of autism using aminosterols. Thus, in one aspect a method of treating, preventing, and/or slowing progression of autism and/or a related symptom in a subject in need is provided, the method comprising administering to the subject a therapeutically effective amount of at least one amino sterol, or a salt or derivative thereof, provided that the administering does not comprise oral administration.

Administration may be via any route of administration other than oral administration. Non-limiting examples include nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof.

The therapeutically effect amount of the at least one aminosterol or a salt or derivative thereof in the methods of the invention can be, for example, about 0.1 to about 20 mg/kg, about 0.1 to about 15 mg/kg, about 0.1 to about 10 mg/kg, about 0.1 to about 5 mg/kg, or about 0.1 to about 2.5 mg/kg body weight of the subject. In another aspect, the therapeutically effect amount of the at least one aminosterol or a salt or derivative thereof in the methods of the invention can be, for example, about 0.001 to about 500 mg/day, about 0.001 to about 250 mg/day, about 0.001 to about 125 mg/day, about 0.001 to about 50 mg/day, about 0.001 to about 25 mg/day, or about 0.001 to about 10 mg/day.

In one embodiment, administering comprises nasal administration. Nasal administration may be accomplished via insufflation of solids or powders, or via inhalation of a mist comprising the at least one aminosterol, or a salt or derivative thereof, in a suitable carrier and optionally excipients. Suitable carriers and excipients are known to the skilled artisan and include buffers such as sodium phosphate, sodium citrate, and citric acid; solubilizers such as glycols, small quantities of alcohol, transcutol (diethylene glycol monoethyl ether), medium chain glycerides, labrasol (saturated polyglycolyzed C₈-C₁₀ glyceride), surfactants and cyclodextrins; preservatives such as parabens, phenyl ethyl alcohol, benzalkonium chloride, EDTA (ethylene diaminetetraaceticacid), and benzoyl alcohol; antioxidants such as sodium bisulfite, butylated hydroxytoluene, sodium metabisulfite and tocopherol; humectants such as glycerin, sorbitol and mannitol; surfactants such as polysorbet; bioadhesive polymers such as mucoadhesives; and penetration enhancers such as dimethyl sulfoxide (DMSO).

Nasal administration via inhalation of a mist may employ the use of metered-dose spray pumps. Typical volumes of aminosterol comprising mist, delivered via a single pump of a metered-dose spray pump may be about 20-100 μl, 100-150 μl, or 150-200 μl. Such pumps offer high reproducibility of the emitted dose and plume geometry in in vitro tests. The particle size and plume geometry can vary within certain limits and depend on the properties of the pump, the formulation, the orifice of the actuator, and the force applied.

In some embodiments, administering comprises nasal administration and the therapeutically effective amount of the at least one amino sterol, or a salt or derivative thereof comprises about 0.1 to about 20 mg/kg body weight of the subject. In some embodiments, administering comprises nasal administration and the therapeutically effective amount of the at least one amino sterol, or a salt or derivative thereof comprises about 0.1 to about 5 mg/kg body weight of the subject. In some embodiments, administering comprises nasal administration and the therapeutically effective amount of the at least one aminosterol, or a salt or derivative thereof comprises about 5 to about 10 mg/kg body weight of the subject. In some embodiments, administering comprises nasal administration and the therapeutically effective amount of the at least one amino sterol, or a salt or derivative thereof comprises about 10 to about 15 mg/kg body weight of the subject. In some embodiments, administering comprises nasal administration and the therapeutically effective amount of the at least one aminosterol, or a salt or derivative thereof comprises about 15 to about 20 mg/kg body weight of the subject.

In some embodiments, administering comprises nasal administration and the therapeutically effective amount of the at least one amino sterol, or a salt or derivative thereof comprises about 0.1 to about 20 mg/kg body weight of the subject. In some embodiments, administering comprises nasal administration and the therapeutically effective amount of the at least one amino sterol, or a salt or derivative thereof comprises about 0.1 to about 15 mg/kg body weight of the subject. In some embodiments, administering comprises nasal administration and the therapeutically effective amount of the at least one aminosterol, or a salt or derivative thereof comprises about 0.1 to about 10 mg/kg body weight of the subject. In some embodiments, administering comprises nasal administration and the therapeutically effective amount of the at least one amino sterol, or a salt or derivative thereof comprises about 0.1 to about 5 mg/kg body weight of the subject.

In some embodiments, administering comprises nasal administration and the therapeutically effective amount of the at least one amino sterol, or a salt or derivative thereof comprises about 0.001 to about 2 mg/day. In some embodiments, administering comprises nasal administration and the therapeutically effective amount of the at least one amino sterol, or a salt or derivative thereof comprises about 2 to about 4 mg/day. In some embodiments, administering comprises nasal administration and the therapeutically effective amount of the at least one amino sterol, or a salt or derivative thereof comprises about 4 to about 6 mg/day. In some embodiments, administering comprises nasal administration and the therapeutically effective amount of the at least one amino sterol, or a salt or derivative thereof comprises about 0.001 to about 6 mg/day.

In some embodiments, administering comprises nasal administration and the therapeutically effective amount of the at least one amino sterol, or a salt or derivative thereof comprises about 0.001 to about 4 mg/day. In some embodiments, administering comprises nasal administration and the therapeutically effective amount of the at least one amino sterol, or a salt or derivative thereof comprises about 0.001 to about 2 mg/day. In some embodiments, administering comprises nasal administration and the therapeutically effective amount of the at least one amino sterol, or a salt or derivative thereof comprises about 0.001 to about 1 mg/day.

In another embodiments, administering comprises nasal administration and the therapeutically effective amount of the at least one amino sterol, or a salt or derivative thereof comprises about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6 mg/day.

### III. Methods of Determining and Compositions

Comprising a “Fixed Dose” of an Aminosterol

The present application also relates to the surprising discovery of a method to determine a “fixed dose” of an aminosterol composition useful in treating, preventing, and/or slowing the onset or progression of ASD or a related symptom, where the dose is not age, size, or weight dependent but rather is individually calibrated. The “fixed amino sterol dose” obtained through this method yields highly effective results in treating the symptom(s) based on which the “fixed aminosterol dose” was determined, related symptoms along the “brain-gut” axis, and the underlying ASD. Further, contemplated herein are methods of leveraging this same “fixed dose” method for methods of prevention of ASD.

A. “Fixed Aminosterol Dose”

A “fixed aminosterol dose,” also referred to herein as a “fixed escalated aminosterol dose,” which will be therapeutically effective is determined for each patient by establishing a starting dose of an amino sterol composition and a threshold for improvement of a particular ASD symptom. Following determining a starting amino sterol dosage for a particular patient, the aminosterol dose is then progressively escalated by a consistent amount over consistent time intervals until the desired improvement is achieved; this amino sterol dosage is the “fixed escalated aminosterol dosage” for that particular patient for that particular symptom.

In exemplary embodiments, an orally administered aminosterol dose is escalated every about 3 to about 5 days by about 25 mg until the desired improvement is reached. Symptoms evaluated, along with tools for measuring symptom improvement, may be specifically described below, including but not limited to constipation, sleep disturbances (e.g. REM disturbed sleep, undifferentiated sleep, circadian rhythm disruption or decreased REM sleep latency), or cognitive impairment.

This therapeutically effective “fixed aminosterol dose” is then maintained throughout treatment and/or prevention. Thus, even if the patient goes “off drug” and ceases taking the aminosterol composition, the same “fixed dose” is taken with no ramp up period following re-initiation of amino sterol treatment.

Not to be bound by theory, it is believed that the aminosterol dose is dependent on the severity of nerve damage relating to the symptom establishing the “fixed amino sterol dose” threshold—e.g. for constipation, the dose may be related to the extent of nervous system damage in the patient's gut.

The amino sterol can be administered via any pharmaceutically acceptable means, such as by injection (e.g., IM, IV, or IP), oral, pulmonary, intranasal, etc. Preferably, the aminosterol is administered orally, intranasally, or a combination thereof.

Oral dosage of an amino sterol can range from about 1 to about 500 mg/day, or any amount in-between these two values. Other exemplary dosages of orally administered aminosterols include, but are not limited to, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day.

Intranasal dosages of an aminosterol are much lower than oral dosages of an aminosterol. Examples of such intranasal aminosterol low dosages include, but are not limited to, about 0.001 to about 6 mg/day, or any amount in-between these two values. For example, the low dosage of an intranasal administered amino sterol can be about 0.001, about 0.005, about 0.01, about 0.02, about 0.03, about 0.04, about 0.05, about 0.06, about 0.07, about 0.08, about 0.09, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.1, about 2.2, about 2.3, about 2.4, about 2.5, about 2.6, about 2.7, about 2.8, about 2.9, about 3, about 3.1, about 3.2, about 3.3, about 3.4, about 3.5, about 3.6, about 3.7, about 3.8, about 3.9, about 4, about 4.1, about 4.2, about 4.3, about 4.4, about 4.5, about 4.6, about 4.7, about 4.8, about 4.9, about 5, about 5.1, about 5.2, about 5.3, about 5.4, about 5.5, about 5.6, about 5.7, about 5.8, about 5.9, or about 6 mg/day.

For intranasal (IN) administration, it is contemplated that the aminosterol dosage may be selected such that it would not provide any pharmacological effect if administered by any other route—e.g., a “subtherapeutic” dosage, and, in addition, does not result in negative effects. For example, Amino sterol 1436 is known to have the pharmacological effects of a reduction in food intake and weight loss. Therefore, in the IN methods of the invention, if the amino sterol is Aminosterol 1436 or a salt or derivative thereof, then if the IN Aminosterol 1436 dosage is administered via another route, such as oral, IP, or IV, then the Aminosterol 1436 dosage will not result in a noticeable reduction in food intake or noticeable weight loss. Similarly, squalamine is known to produce the pharmacological effects of nausea, vomiting and/or reduced blood pressure. Thus, in the IN methods of the invention, if the aminosterol is squalamine or a salt or derivative thereof, then if the IN squalamine dosage is administered via another route, such as oral, IP, or IV, then the squalamine dosage will not result in noticeable nausea, vomiting, and/or a reduction in blood pressure. Suitable exemplary aminosterol dosages are described above.

Dose Escalation:

When determining a “fixed aminosterol dosage” for a particular patient, a patient is started at a lower dose and then the dose is escalated until a positive result is observed for the symptom being evaluated. For example, constipation is exemplified in Example 1. Aminosterol doses can also be de-escalated (reduced) if any given aminosterol dose induces a persistent undesirable side effect, such as diarrhea, vomiting, or nausea.

The starting aminosterol dose is dependent on the severity of the symptom—e.g. for a patient experiencing severe constipation, defined as less than one spontaneous bowel movement (SBM) a week, the starting oral aminosterol dose can be about 150 mg/day or greater. In contrast, for a patient having moderate constipation, e.g., defined as having more than one SBM a week, the starting amino sterol dose can be about 75 mg/day. Thus, as an example, a patient experiencing moderate constipation can be started at an amino sterol dosage of about 75 mg/day, whereas a patient experiencing severe constipation can be started at an aminosterol dosage of about 150 mg/day.

In other embodiments, a patient experiencing moderate symptoms (for the symptom being used to calculate a fixed escalated aminosterol dose) can be started at an oral aminosterol dosage of from about 10 mg/day to about 75 mg/day, or any amount in-between these values. For example, the starting oral amino sterol dosage for a moderate symptom can be about 10, about 15, about 20, about 25, about 30, about 35, about 40, about 45, about 60, about 65, about 70, or about 75 mg/day.

In yet further embodiments, when the patient is experiencing severe symptoms (for the symptom being used to calculate the fixed escalated aminosterol dose), the patient can be started at an oral aminosterol dosage ranging from about 75 to about 175 mg/day, or any amount in-between these two values. For example, the starting oral aminosterol dosage for a severe symptom can be about 75, about 80, about 85, about 90, about 95, about 100, about 105, about 110, about 115, about 120, about 125, about 130, about 135, about 140, about 145, about 150 about 155, about 160, about 165, about 170, or about 175 mg/day.

In some embodiments, the starting oral aminosterol dose may be about 125 mg/day or about 175 mg/day; again dependent on the severity of the symptom, such as constipation.

Starting IN aminosterol dosages prior to dose escalation can be, for example, about 0.001 mg to about 3 mg/day, or any amount in-between these two values. For example, the starting aminosterol dosage for IN administration, prior to dose escalation, can be, for example, about 0.001, about 0.005, about 0.01, about 0.02, about 0.03, about 0.05, about 0.06, about 0.07, about 0.08, about 0.09, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 1.0, about 1.1, about 1.25, about 1.3, about 1.4, about 1.5, about 1.6, about 1.7, about 1.75, about 1.8, about 1.9, about 2.0, about 2.1, about 2.25, about 2.3, about 2.4, about 2.5, about 2.6, about 2.7, about 2.75, about 2.8, about 2.9, or about 3 mg/day.

In exemplary embodiments, the aminosterol dose is given periodically as needed. For example, the aminosterol dose can be given once per day. The amino sterol dose can also be given every other day, 2, 3, 4, or 5× per week, once/week, or 2×/week. In another embodiment, the aminosterol dose can be given every other week, or it can be administered for a first period of time of administration, followed by a cessation of administration for a second period of time, followed by resuming administration upon recurrence of autism or a symptom of autism.

When calculating a fixed escalated aminosterol dose, the dose can be escalated following any suitable period of time. In one embodiment, the aminosterol dose is escalated every about 3 to about 7 days by about a defined amount until a desired improvement is reached. For example, when the symptom being treated/measured is constipation, threshold improvement can be an increase of one SBM per week or at least a total of three bowel movements per week. In other embodiments, the aminosterol dose can be escalated every about 1, about 2, about 3, about 4, about 5, about 6, about 7, about 8, about 9, about 10, about 11, about 12, about 13, or about 14 days. In other embodiments, the amino sterol dose can be escalated about 1×/week, about 2×/week, about every other week, or about 1×/month.

During dose escalation, the aminosterol dosage can be increased by a defined amount. For example, when the aminosterol is administered orally, the dose can be escalated in increments of about 5, about 10, about 15, about 20, about 25, about 30, about 35, about 40, about 45, or by about 50 mg. When the aminosterol is administered intranasally, then the dosage can be increased in increments of about, for example, about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg.

Other symptoms that can be used as an endpoint to determine aminosterol dosage for a patient's fixed escalated aminosterol dosage are described herein and include, but are not limited to, (a) a symptom from the Autism Spectrum Rating Scales (ASRS™) selected from the group consisting of social skills, communication skills, unusual behavior, self-regulation ability, peer socialization, adult socialization, atypical language, and stereotypy; (b) a symptom from the Autism Diagnostic Observation Schedule (ADOS) selected from the group consisting of performance in Module 1, performance in Module 2, performance in Module 3, and performance in Module 4; (c) a symptom from the Autism Diagnostic Interview-Revised (ADI-R) wherein the symptom is selected from the group consisting of emotional sharing, offering and seeking comfort, social smiling, responding to other children, stereotyped utterances, pronoun reversal, social usage of language, preoccupation with unusual things, hand and finger mannerism, unusual sensory interests, self-injury, aggression, and overactivity; (d) failure to respond to name; (e) failure to point at objects of interest; (f) inability to role play; (g) avoidance of eye contact; (h) preference to be alone; (i) inability to understand feelings of others; (j) no speech or delayed development of speech; (k) echolalia and/or palilalia; (l) answering questions with unrelated answers; (m) upset by minor changes; (n) obsessive interests; (o) lining-up or stacking of objects; (p) repetitive motion; (q) avoidance of physical contact with others; (r) lack of awareness of danger; (s) sleep disorder or sleep disturbance; (r) constipation; (s) cognitive impairment; (t) gastrointestinal (GI) problems; (u) epilepsy (observed in up to a third of the autism population); (v) feeding issues; (w) Attention-deficit/hyperactivity disorder (ADHD); (x) anxiety; (y) depression; (z) Obsessive compulsive disorder (OCD); (aa) schizophrenia; and (bb) Bipolar Disorder. Examples of GI issues/disorders include, but are not limited to, chronic constipation, abdominal pain, gastroesophageal reflux, and bowel inflammation.

B. Aminosterols

U.S. Pat. No. 6,962,909, entitled “Treatment of neovascularization disorders with squalamine,” discloses various aminosterols, and this disclosure is specifically incorporated by reference with respect to its teaching of aminosterol compounds. Any aminosterol known in the art, including those described in U.S. Pat. No. 6,962,909, can be used in the disclosed compositions. In some embodiments, the aminosterol present in the compositions of the invention is Aminosterol 1436 or a salt or derivative thereof, squalamine or a salt or derivative thereof, or a combination thereof. An exemplary salt is a phosphate salt.

For instance, useful aminosterol compounds comprise a bile acid nucleus and a polyamine, attached at any position on the bile acid, such that the molecule exhibits a net positive charge contributed by the polyamine.

Thus, in some embodiments, the disclosed methods comprise administering a therapeutically effective amount of one or more aminosterols having the chemical structure of Formula I:

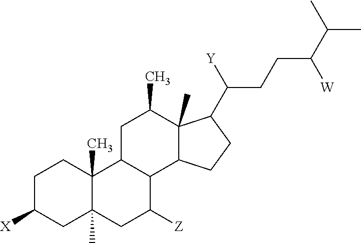

wherein,

W is 24S—OSO₃ or 24R—OSO₃;

X is 3β-H₂N—(CH₂)₄—NH—(CH₂)₃—NH— or 3α-H₂N—(CH₂)₄—NH—(CH₂)₃—NH—;

Y is 20R—CH₃; and

Z is 7α or 7β-OH.

In another embodiment of the invention, the amino sterol is one of the naturally occurring aminosterols (1-8) isolated from Squalus acanthias:

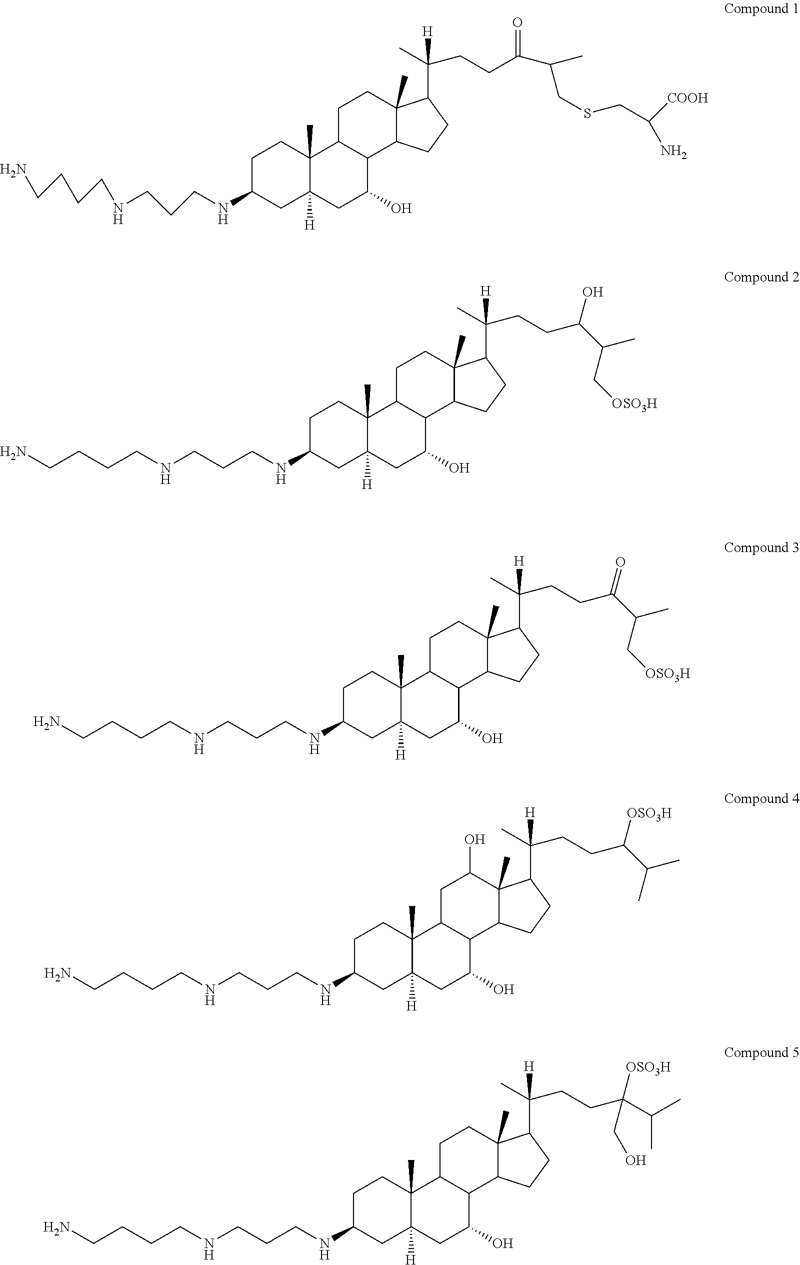

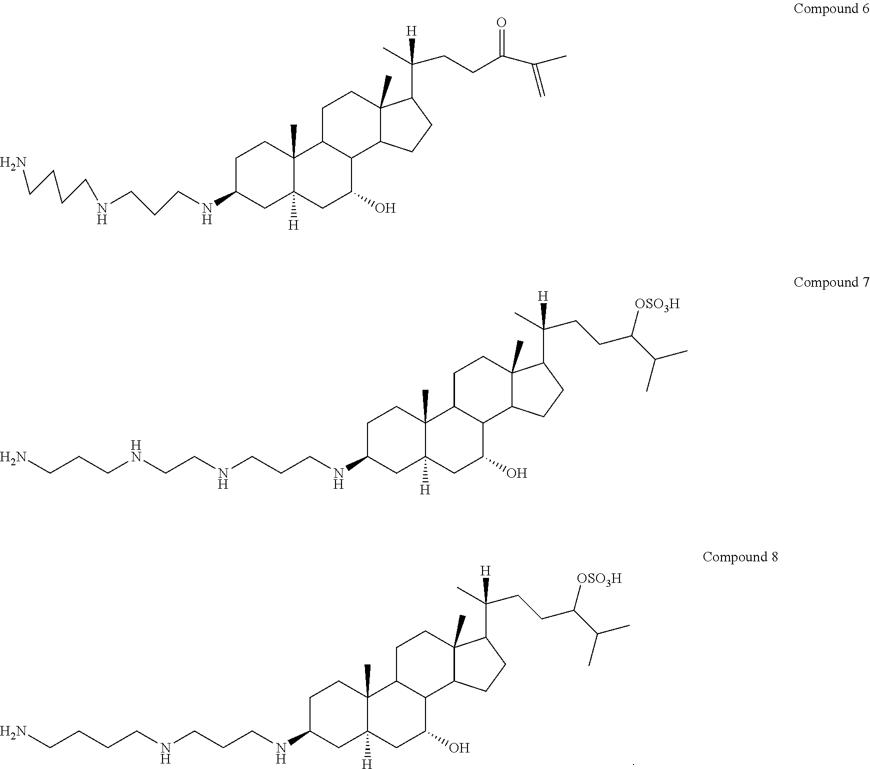

In one aspect of the invention, the aminosterol is Aminosterol 1436 or a salt or derivative thereof or squalamine or a salt or derivative thereof.

Variants or derivatives of known aminosterols, such as squalamine, Aminosterol 1436, or an aminosterol isolated from Squalus acanthias, may be used in the disclosed compositions and methods.

In one embodiment, the amino sterol is a derivative of squalamine, amino sterol 1436, or another naturally occurring aminosterol modified through medical chemistry to improve biodistribution, ease of administration, metabolic stability, or any combination thereof. In another embodiment, the aminosterol is modified to include one or more of the following: (1) substitutions of the sulfate by a sulfonate, phosphate, carboxylate, or other anionic moiety chosen to circumvent metabolic removal of the sulfate moiety and oxidation of the cholesterol side chain; (2) replacement of a hydroxyl group by a non-metabolizable polar substituent, such as a fluorine atom, to prevent its metabolic oxidation or conjugation; and (3) substitution of various ring hydrogen atoms to prevent oxidative or reductive metabolism of the steroid ring system.

In yet another embodiment, the aminosterol comprises a sterol nucleus and a polyamine, attached at any position on the sterol, such that the molecule exhibits a net charge of at least +1, the charge being contributed by the polyamine.

In yet another embodiment, the aminosterol comprises a bile acid nucleus and a polyamine, attached at any position on the bile acid, such that the molecule exhibits a net positive charge being contributed by the polyamine.

In some embodiments, the compositions used in the methods of the invention comprise: (a) at least one pharmaceutical grade amino sterol; and optionally (b) at least one phosphate selected from the group consisting of an inorganic phosphate, an inorganic pyrophosphate, and an organic phosphate. In some embodiments, the aminosterol is formulated as a weakly water soluble salt of the phosphate. In some embodiments, the phosphate is an inorganic polyphosphate, and the number of phosphates can range from about 3 (tripolyphosphate) to about 400, or any number in-between these two values. In other embodiments, the phosphate is an organic phosphate which comprises glycerol 2 phosphates.

In some embodiments, the amino sterol is selected from the group consisting of: (a) squalamine or a pharmaceutically acceptable salt or derivative thereof; (b) a squalamine isomer; (c) Aminosterol 1436; (d) an aminosterol comprising a sterol or bile acid nucleus and a polyamine, attached at any position on the sterol or bile acid, such that the molecule exhibits a net charge of at least +1, the charge being contributed by the polyamine; (e) an aminosterol which is a derivative of squalamine modified through medical chemistry to improve biodistribution, ease of administration, metabolic stability, or any combination thereof; (f) an aminosterol modified to include one or more of the following: (i) substitutions of the sulfate by a sulfonate, phosphate, carboxylate, or other anionic moiety chosen to circumvent metabolic removal of the sulfate moiety and oxidation of the cholesterol side chain; (ii) replacement of a hydroxyl group by a non-metabolizable polar substituent, such as a fluorine atom, to prevent its metabolic oxidation or conjugation; and (iii) substitution of various ring hydrogen atoms to prevent oxidative or reductive metabolism of the steroid ring system; (g) an aminosterol that can inhibit the formation of actin stress fibers in endothelial cells stimulated by a ligand known to induce stress fiber formation, having the chemical structure of Formula I (above); or (h) any combination thereof.

In some embodiments, the methods of the invention can employ a formulation of Aminosterol 1436 or squalamine as an insoluble salt of phosphate, polyphosphate, or an organic phosphate ester.

Any pharmaceutically acceptable salt of an amino sterol can be used in the compositions and methods of the invention. For example, a phosphate salt or buffer, free base, succinate, phosphate, mesylate or other salt form associated with low mucosal irritation can be utilized in the methods and compositions of the invention.

C. Routes of Administration

It is appreciated that the “fixed aminosterol dose” disclosed herein can be administered via any suitable route of administration, including but not limited to oral or intranasal delivery, injection (IP, IV, or IM) or a combination thereof.

Further, co-administration of the “fixed dose” with injectable (e.g., IP, IV, IM) aminosterol formulations is also contemplated herein. For injectable dosage forms, the dosage form can comprise an aminosterol at a dosage of, for example, about 0.1 to about 20 mg/kg body weight. In other embodiments, the effective daily dosing amount is about 0.1, about 0.5, about 1, about 2, about 3, about 4, about 5, about 6, about 7, about 8, about 9, about 10, about 11, about 12, about 13, about 14, about 15, about 16, about 17, about 18, about 19, or about 20 mg/kg body weight.

The invention also encompasses methods of treatment using a combination of an aminosterol composition administered via one route, e.g., oral, with a second amino sterol composition, comprising the same or a different amino sterol, administered via a different route, e.g., intranasal. For example, in a method of the invention, squalamine can be administered orally and aminosterol 1436 can be administered IN.

D. Dosing Period

The pharmaceutical composition comprising an aminosterol or a derivative or salt thereof can be administered for any suitable period of time, including as a maintenance dose for a prolonged period of time. Dosing can be done on an as needed basis using any pharmaceutically acceptable dosing regimen. Aminosterol dosing can be no more than 1× per day, once every other day, once every three days, once every four days, once every five days, once every six days, once a week, or divided over multiple time periods during a given day (e.g., twice daily).

In other embodiments, the composition can be administered: (1) as a single dose, or as multiple doses over a period of time; (2) at a maintenance dose for an indefinite period of time; (3) once, twice or multiple times; (4) daily, every other day, every 3 days, weekly, or monthly; (5) for a period of time such as about 1, about 2, about 3, or about 4 weeks, about 1, about 2, about 3, about 4, about 5, about 6, about 7, about 8, about 9, about 10, about 11, or about 12 months, about 1 year, about 1.5 years, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5, about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, about 24, about 24.5, or about 25 years, or (6) any combination of these parameters, such as daily administration for 6 months, weekly administration for 1 or more years, etc.

Yet another exemplary dosing regimen includes periodic dosing, where an effective dose can be delivered once every about 1, about 2, about 3, about 4, about 5, about 6 days, or once weekly.

In a preferred embodiment, the aminosterol dose is taken in the morning, i.e. on an empty stomach preferably within about two hours of waking up and may be followed by a period without food, such as for example about 60 to about 90 minutes. In other embodiments, the aminosterol dose is taken within about 15 min, about 30 min, about 45 min, about 1 hr, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs within waking up. In yet further embodiments, the aminosterol dose is followed by about period without food, wherein the period is at least about 30 min, about 45 mins, about 60 mins, about 1.25 hrs, about 1.5 hrs, about 1.75 hrs, or about 2 hrs.

Not to be bound by theory, it is believed that since aminosterols have an impact on circadian rhythms, likely due to ENS signaling thereof, taking the amino sterol dose in the morning enables the synchronization of all the autonomic physiological functions occurring during the day. In other embodiments of the invention, the amino sterol dosage is taken within about 15 mins, about 30 mins, about 45 mins, about 1 hour, about 1.25 hrs, about 1.5 hrs, about 1.75 hrs, about 2 hrs, about 2.25 hrs, about 2.5 hrs, about 2.75 hrs, about 3 hrs, about 3.25 hrs, about 3.5 hrs, about 3.75 hrs, or about 4 hrs of waking up. In addition, in other embodiments of the invention, following the aminosterol dosage the subject has a period of about 15 mins, about 30 mins, about 45 mins, about 1 hours, about 1.25 hrs, about 1.5 hrs, about 1.75 hrs, about 2 hrs, about 2.25 hrs, about 2.5 hrs, about 2.75 hrs, or about 3 hours without food.

In some embodiments, each defined period of time is independently selected from the group consisting of about 1 day to about 10 days, about 10 days to about 30 days, about 30 days to about 3 months, about 3 months to about 6 months, about 6 months to about 12 months, and about greater than 12 months.

E. Composition Components

In some embodiments, a pharmaceutical composition disclosed herein comprises one or more pharmaceutically acceptable carriers, such as an aqueous carrier, buffer, and/or diluent.

In some embodiments, a pharmaceutical composition disclosed herein further comprises a simple polyol compound, such as glycerin. Other examples of polyol compounds include sugar alcohols. In some embodiments, a pharmaceutical composition disclosed herein comprises an aqueous carrier and glycerin at about a 2:1 ratio.

The formulations may conveniently be presented in unit dosage form and may be prepared by any of the methods well known in the art of pharmacy. An exemplary oral dosage form is a tablet or capsule. An exemplary intranasal dosage form is a liquid or powder nasal spray. A nasal spray is designed to deliver drug to the upper nasal cavity, and can be a liquid or powder formulation, and in a dosage form such as an aerosol, liquid spray, or powder.

The amino sterol may be combined or coordinately administered with a suitable carrier or vehicle depending on the route of administration. As used herein, the term “carrier” means a pharmaceutically acceptable solid or liquid filler, diluent or encapsulating material. A water-containing liquid carrier can comprise pharmaceutically acceptable additives such as acidifying agents, alkalizing agents, antimicrobial preservatives, antioxidants, buffering agents, chelating agents, complexing agents, solubilizing agents, humectants, solvents, suspending and/or viscosity-increasing agents, tonicity agents, wetting agents or other biocompatible materials. A tabulation of ingredients listed by the above categories can be found in the U.S. Pharmacopeia National Formulary, 1857-1859, and (1990). Some examples of the materials which can serve as pharmaceutically acceptable carriers are sugars, such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols, such as propylene glycol; polyols such as glycerin, sorbitol, mannitol and polyethylene glycol; esters such as ethyl oleate and ethyl laurate; agar; buffering agents such as magnesium hydroxide and aluminum hydroxide; alginic acid; pyrogen free water; isotonic saline; Ringer's solution, ethyl alcohol and phosphate buffer solutions, as well as other nontoxic compatible substances used in pharmaceutical formulations. Wetting agents, emulsifiers and lubricants such as sodium lauryl sulfate and magnesium stearate, as well as coloring agents, release agents, coating agents, sweetening, flavoring and perfuming agents, preservatives and antioxidants can also be present in the compositions, according to the desires of the formulator. Examples of pharmaceutically acceptable antioxidants include water soluble antioxidants such as ascorbic acid, cysteine hydrochloride, sodium bisulfite, sodium metabisulfite, sodium sulfite and the like; oil-soluble antioxidants such as ascorbyl palmitate, butylated hydroxyanisole (BHA), butylated hydroxytoluene (BHT), lecithin, propyl gallate, alpha-tocopherol and the like; and metal-chelating agents such as citric acid, ethylenediamine tetraacetic acid (EDTA), sorbitol, tartaric acid, phosphoric acid and the like.

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art. Examples of filling agents include lactose monohydrate, lactose anhydrous, and various starches; examples of binding agents include various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™). Suitable lubricants, including agents that act on the flowability of the powder to be compressed, may include colloidal silicon dioxide, such as Aerosil® 200, talc, stearic acid, magnesium stearate, calcium stearate, and silica gel. Examples of sweeteners may include any natural or artificial sweetener, such as sucrose, xylitol, sodium saccharin, cyclamate, aspartame, and acesulfame. Examples of flavoring agents are Magnasweet® (trademark of MAFCO), bubble gum flavor, and fruit flavors, and the like. Examples of preservatives include potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quaternary compounds such as benzalkonium chloride.

Any pharmaceutical used for therapeutic administration can be sterile. Sterility is readily accomplished by for example filtration through sterile filtration membranes (e.g., 0.2 micron membranes). Any pharmaceutically acceptable sterility method can be used in the compositions of the invention.

The pharmaceutical composition comprising an aminosterol derivatives or salts thereof will be formulated and dosed in a fashion consistent with good medical practice, taking into account the clinical condition of the individual patient, the method of administration, the scheduling of administration, and other factors known to practitioners.

F. Kits

Aminosterol formulations or compositions of the invention may be packaged together with, or included in a kit along with instructions or a package insert. Such instructions or package inserts may address recommended storage conditions, such as time, temperature and light, taking into account the shelf-life of the aminosterol or derivatives or salts thereof. Such instructions or package inserts may also address the particular advantages of the aminosterol or derivatives or salts thereof, such as the ease of storage for formulations that may require use in the field, outside of controlled hospital, clinic or office conditions.

The invention also provides a pharmaceutical pack or kit comprising one or more containers filled with one or more amino sterol pharmaceutical compositions disclosed herein. The kits may include, for instance, containers filled with an appropriate amount of an aminosterol pharmaceutical composition, either as a powder, a tablet, to be dissolved, or as a sterile solution. Associated with such container(s) can be a notice in the form prescribed by a governmental agency regulating the manufacture, use or sale of pharmaceuticals or biological products, which notice reflects approval by the agency of manufacture, use or sale for human administration. In addition, the aminosterol or a derivative or salt thereof may be employed in conjunction with other therapeutic compounds.

In other aspects, a kit comprising a nasal spray device as described herein is disclosed. In one aspect, the kit may comprise one or more devices as disclosed herein, comprising a disclosed low dose aminosterol composition, wherein the device is sealed within a container sufficient to protect the device from atmospheric influences. The container may be, for example, a foil, or plastic pouch, particularly a foil pouch, or heat sealed foil pouch. Suitable containers sufficient to adequately protect the device will be readily appreciated by one of skill in the art.

In one aspect, the kit may comprise one or more devices as disclosed herein, wherein the device may be sealed within a first protective packaging, or a second protective packaging, or a third protective packaging, that protects the physical integrity of the product. One or more of the first, second, or third protective packaging may comprise a foil pouch. The kit may further comprise instructions for use of the device. In one aspect, the kit contains two or more devices.

In one aspect, the kit may comprise a device as disclosed herein, and may further comprise instructions for use. In one aspect, the instructions may comprise visual aid/pictorial and/or written directions to an administrator of the device.

G. Patient Populations

The disclosed compositions can be used to treat a range of subjects, including human and non-human animals, including mammals, as well as immature and mature animals, including human children and adults. The human subject to be treated can be an infant, toddler, school-aged child, teenager, young adult, adult, or elderly patient.

In embodiments disclosed herein relating to prevention, particular patient populations may be selected based on being “at risk for” the development of one or more ASD disorders. For example, family history may be used as signs to identify subjects likely to develop ASD. Thus, in some embodiments relating to disorders for which certain genetic or hereditary signs are known, prevention may involve first identifying a patient population based on one of the signs. Alternatively, certain symptoms are considered early signs of particular disorders. Thus, in some embodiments, a patient population may be selected for being “at risk” for developing ASD based on age and experiencing constipation. Further genetic or hereditary signs may be used to refine the patient population, such as having a parent or sibling diagnosed with ASD.

Patient populations may also be identified based on the presence of one or more genes known to increase the risk of ASD. In 2014, researchers identified 60 genes with more than a 90% chance of increasing the risk of autism in a child. In addition, in 2017, an analysis published in Nature Neuroscience identified 18 new genes linked with autism.

Further, recent reports indicate that brain scans, e.g., MRIs, and artificial intelligence can predict with extremely high accuracy (96%) which 6 month old infants would go on to be diagnosed with autism as toddlers. R. Emerson 2017. Thus, such techniques can be used to screen and identify subjects who can benefit from the methods of the invention. For example, patient population to be treated with the methods of the invention can be less than about 1 year, less than about 1.5 years, less than about 2 years, less than about 2.5 years, less than about 3 years, less than about 3.5 years, less than about 4 years, less than about 4.5 years, less than about 5 years, less than about 5.5 years, less than about 6 years, less than about 7 years, less than about 8 years, less than about 10 years, less than about 15 years, less than about 20 years, or an adult of about 20 years or older.

### IV. Methods of ASD Prevention and/or Treatment with a “Fixed Dose” of Aminosterol

Aspects of this disclosure relate to methods of treating, preventing, and/or slowing the progression or onset of ASD and/or a related symptom by administration of a “fixed dose” of aminosterol as disclosed herein. As noted herein, one or more of the symptoms disclosed herein can be used to determine the fixed dose of the aminosterol or a salt or derivative thereof during the dose escalation process.

Example 1 provides a detailed protocol for determining a “fixed dose” based on improvement of one symptom associated with Parkinson's disease (PD), e.g., constipation. This example further details how this “fixed dose” successfully treated not only constipation, but also other non-dopamine related symptoms of PD, which therefore are applicable to treatment of ASD.

As dopaminergic activity distinguishes PD from other neurodegenerative disorders and these data relate to symptoms that do not relate to this distinguishing feature, this dosing regime is believed to be extrapolatable both to other symptoms and other disorders including ASD.

Not to be bound by theory, it is believed that establishing a patient-specific “fixed dose” based on hitting a threshold improvement in any of the symptoms listed below and administering this therapeutically effective fixed dose will successfully treat the initial symptom and one or more of the other symptoms. Further, to the extent that these symptoms are tied to an underlying disorder, administration of the therapeutically effective fixed dose is also believed to offer a means of treating, preventing, and/or delaying onset of the underlying disorder.

B. ASD Symptoms

I. Constipation

While often dismissed as strictly a gastrointestinal symptom, constipation is believed to be an early indicator of neurodisease to the extent that ENS degeneration can be indicative of later CNS degeneration. Indeed, not to be bound by theory, but constipation is observed in patients with ASD. Accordingly, method embodiments disclosed herein relate to the treatment of constipation or the treatment and/or prevention of an underlying disorder associated with constipation.

Constipation is defined as a lower than normal frequency of bowel movements in a fixed duration of time (e.g. less than 3 bowel movements per week). Constipation not only constitutes a major economic burden, but it also significantly affects the quality of life of the individual, contributing to social isolation and depression. Furthermore, the severity of the symptoms correlates negatively with patient reported quality of life.

Example 1 describes several tools used to measure and evaluate the effect of amino sterol treatment on constipation, including for example:

(1) Rome-IV Criteria for Constipation (7 criteria, with constipation diagnosis requiring two or more of the following: (i) straining during at least 25% of defecations, (ii) lumpy or hard stools in at least 25% of defecations, (iii) sensation of incomplete evacuation for at least 25% of defecations, (iv) sensation of anorectal obstruction/blockage for at least 25% of defecations; (v) manual maneuvers to facilitate at least 25% of defecations; (vi) fewer than 3 defecations per week; and (vii) loose stools are rarely present without the use of laxatives;

(2) Constipation—Ease of Evacuation Scale (from 1-7, with 7=incontinent, 4=normal, and 1=manual disimpaction);

(3) Bristol Stool Chart, which is a patient-friendly means of categorizing stool characteristics (assessment of stool consistency is a validated surrogate of intestinal motility) and stool diary;

(4) Unified Parkinson's Disease Scale (UPSRS), section 1.11 (Constipation Problems);

(5) Patient Assessment of Constipation Symptoms (PAC-SYM); and

(5) Patient Assessment of Constipation Quality of Life (PAC-QOL).

Examples of characteristics of constipation that can be positively affected by the method of the invention include, but are not limited to, frequency of constipation, duration of constipation symptoms, bowel movement frequency, stool consistency, abdominal pain, abdominal bloating, incomplete evacuation, unsuccessful attempts at evacuation, pain with evacuation, and straining with evacuation. Potentially all of these characteristics can be positively impacted by the methods of the invention. Further, assessments of these characteristics are known in the art, e.g. spontaneous bowel movements (SBMs)/week, stool consistency (Bristol Stool Form Scale) (Lewis and Heaton 1997; Heaton et al. 1992), ease of passage (Ease of Evacuation Scale) (Andresen et al. 2007), rescue medication use and symptoms and quality of life related to bowel function (PAC-SYM (Frank et al. 1999) and PAC-QOL (Marquis et al. 2005)).

The methods of using a therapeutically effective fixed dose of an aminosterol composition according to the invention to treat and/or prevent constipation associated with ASD preferably results in an increase in the number of spontaneous bowel movements per week and/or an improvement in other stool conditions. The increase can be, for example, an increase of between 1 to 3 spontaneous bowel movements in a week, or, optionally, full restoration of regular bowel function.

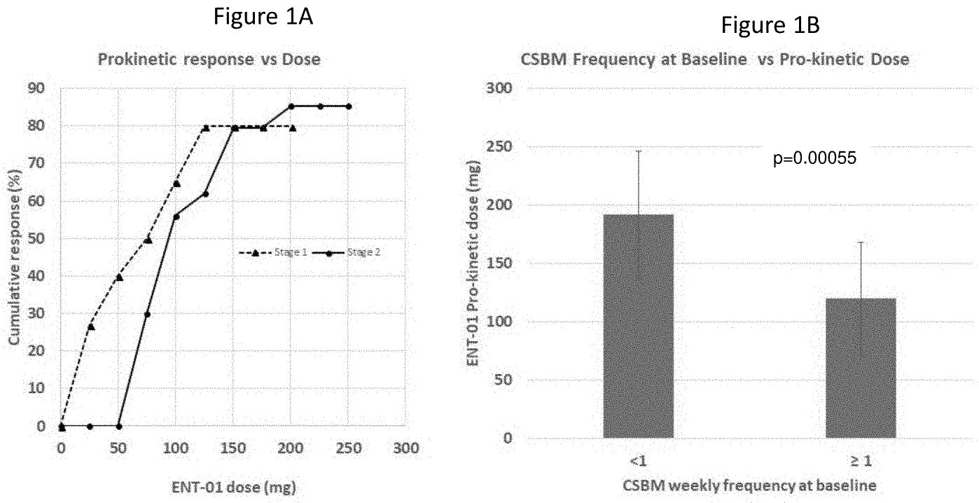

Data detailed in Example 1 shows that 80% of subjects responded to aminosterol treatment with improved bowel function (see FIG. 1A), with the cumulative response rate increasing in a dose-dependent fashion from 25% at 25 mg to a maximum of 80% at 200 mg (Stage 1, FIG. 1A). In Stage 2 of the study, the response rate increased in a dose-dependent fashion from 26% at 75 mg to 85.3% at 250 mg (FIG. 1A). The dose required for a bowel response was patient-specific and varied from 75 mg to 250 mg. The median efficacious dose was 100 mg.

The average CSBM/week increased from 1.2 at baseline to 3.8 at fixed dose (216% improvement) and SBM increased from 2.6 at baseline to 4.5 at fixed dose (73% improvement). Use of rescue medication decreased from 1.8/week at baseline to 0.3 at fixed dose (83% decrease). Consistency based on the Bristol stool scale also improved, increasing from mean 2.7 to 4.1 (52% improvement) and ease of passage increased from 3.2 to 3.7 (16% improvement). Subjective indices of wellbeing (PAC-QOL) and constipation symptoms (PAC-SYM) also improved during treatment.

The dose that proved efficacious in inducing a bowel response was strongly related to constipation severity at baseline (FIG. 1B); patients with baseline constipation of <1 CSBM/week required higher doses for a response (mean 192 mg) than patients with ≥1 CSBM/week (mean 120 mg).

In one embodiment of the invention, treatment of an ASD subject having constipation with an aminosterol in a method described herein results in an improvement of one or more characteristics of constipation associated with ASD. The improvement can be, for example, about 5, about 10, about 15, about 20, about 25, about 30, about 35, about 40, about 45, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290, about 300, about 325, about 350, about 375 or about 400%. Examples of constipation characteristics that can be improved by the methods of the invention include, but are not limited to, frequency of constipation, duration of constipation symptoms, bowel movement frequency, stool consistency, abdominal pain, abdominal bloating, incomplete evacuation, unsuccessful attempts at evacuation, pain with evacuation, and straining with evacuation. Measurement of a constipation characteristic can be done using any clinically recognized scale or tool.

One surprising discovery that resulted from the experiments described herein related to aminosterol dosing. It was surprisingly discovered that the dose of aminosterol required to obtain a positive impact on a symptom being evaluated, referred to herein as a “fixed escalated aminosterol dose,” is patient specific. Moreover, it was discovered that the fixed escalated aminosterol dose is not dependent upon age, size, or weight but rather is individually calibrated. Further, it was discovered that the severity of constipation correlates with a higher required “fixed escalated aminosterol dose.” It is theorized that the aminosterol dose required to obtain a positive effect in a subject for the symptom being evaluated correlates with the extent of neuronal damage. Thus, it is theorized that greater neuronal damage correlates with a higher required aminosterol dose to obtain a positive effect in a subject for the symptom being evaluated. The observation that the aminosterol dose required to achieve a desired response increases with constipation severity supports the hypothesis that the greater the burden of αS impeding neuronal function, the higher the dose of aminosterol required to restore normal bowel function. Moreover, the data described in Example 1 confirms the hypothesis that gastrointestinal dysmotility in PD results from the progressive accumulation of αS in the ENS, and that amino sterol treatment can restore neuronal function by displacing αS and stimulating enteric neurons. These results demonstrate that the ENS in PD is not irreversibly damaged and can be restored to normal function.

In calibrating the fixed aminosterol dose for a specific ASD patient, the starting dose is varied based upon the severity of the constipation (when constipation is used as the ASD symptom to be evaluated). Thus, for subjects with severe constipation, e.g., subjects with 1 or less CSBM or SMB per week, oral aminosterol dosing is started at about 100 to about 175 mg or more (or any amount in-between these values as described herein). For subjects with less severe constipation, e.g., more than 1 CSBM or SBM per week, oral aminosterol dosing is started at about 25 to about 75 mg (or any amount in-between these values as described herein). Dosing for both patients is then escalated by defined amounts over a defined period of time until the fixed escalated dose for the patient is identified. Aminosterol doses can also be de-escalated (reduced) if any given amino sterol dose induces a persistent undesirable side effect, such as diarrhea, vomiting, or nausea.

For example, for ASD patients with severe constipation, a starting oral amino sterol dosage can be from 75 mg up to about 300 mg, or any amount in-between these two values. In other embodiments, the starting oral aminosterol dosage for severely constipated patients can be, for example, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, or about 300 mg. A “fixed escalated” oral aminosterol dose for a severely constipated patient is likely to range from about 75 mg up to about 500 mg. As described in Example 1, a positive effect was defined as a dose that resulted in a CSBM within 24 hours of dosing on at least 2 of 3 days at a given dose.

For ASD patients with less severe constipation, oral aminosterol dosing is started at about 10 to about 75 mg, or any amount in-between these two values as described herein. For example, starting oral aminosterol dosage for patients with moderate to mild constipation can be about 1, about 5, about 10, about 15, about 20, about 25, about 30, about 35, about 40, about 45, about 50, about 55, about 60, about 65, about 70, up to less than or equal to about 75 mg. A fixed escalated oral aminosterol dose for a mild or moderately constipated patient is likely to range from about 5 mg up to about 350 mg, or any amount in-between these two values as described herein.

2. Sleep Disturbance/Sleep Problems Associated with ASD (e.g., Decreased REM Sleep or Decreased REM Sleep Latency)

Sleep disturbances can be associated with ASD. Normal sleep is critically important for the proper functioning of many organ systems, the most important of which is the brain. Disturbances in normal sleep patterns are closely associated with the normal aging process, with the development of cognitive impairment, with impaired memory deposition and consolidation and with the occurrence of neurodevelopmental, neuroaffective and neurodegenerative disorders. The alternating pattern of sleep and wakefulness occurring every 24 hours is known as the circadian rhythm. The rhythm is set by the “Zeitgeber” (time setter), an entity known as the suprachiasmatic nucleus (SCN) and located in the hypothalamus. The SCN is normally “entrained” or synchronized by the external light-dark cycle. This relationship between external light and dark and the sleep wake cycle synchronized to it by the SCN can be over ridden during periods of hunger by neural signals emanating in the gut and relayed to the hypothalamus. The circadian sleep-wake cycle can also shift in response to changes in external light-dark cycles, such as the desynchronization that occurs during travel from one time zone to another (jet-lag). Under such circumstances, a progressive adjustment occurs until the SCN is resynchronized with the external light-dark cycle. A similar “phase-shift” and adjustment occurs in night-shift workers.

Under normal circumstances, the properly functioning SCN, synchronized to the external light-dark cycle and to neural signals emanating from the enteric nervous system, will regulate the sleep-wake cycle by sending neural and chemical signals to the surrounding structures and to portions of the brain stem involved in sleep and wakefulness. An individual with a properly functioning hypothalamus and brain stem will go to bed and fall asleep within minutes, remain asleep throughout the night, wake up in the morning and remain awake and alert throughout the day. During the night, the asleep individual will experience several cycles of sleep, beginning with light sleep, progressing through rapid eye movement sleep (REM-sleep) to deep sleep and back. Each complete sleep period lasts about 90 minutes. Periods of REM-sleep are closely associated with dreaming. During REM-sleep, neural signals emanating from certain parts of the brain stem ensure that skeletal muscles become “atonic” or are paralyzed, such that the individual can't “act out” their dreams.

Certain diseases and conditions may impair the normal functioning of the “Zeitgeber” or circadian clock, including ASD. These conditions may be reversible, such as desynchronization resulting from ASD.

Dysfunction of the circadian rhythm manifests first and foremost by abnormal sleep patterns. Such abnormalities typically are mild at onset and worsen progressively over time. A common symptom of sleep disorder is a delay in the onset of sleep. This delay can be as long as several hours, and the individual may not be able to fall asleep until the early hours of the morning. Another common symptom is sleep fragmentation, meaning that the individual awakens several times during the course of the night. Once awakened, the individual may not be able to get back to sleep, and each awake fragment may last an hour or more, further reducing “total sleep time,” which is calculated by subtracting total time of the awake fragments from total time spent in bed. Total sleep time also diminishes with age, from about 14 to about 16 hours a day in newborns, to about 12 hours by one year of age, to about 7 to about 8 hours in young adults, progressively declining to about 5 to about 6 hours in elderly individuals. Total sleep time can be used to calculate an individual's “sleep age” and to compare it to their chronologic age. Significant discrepancies between sleep age and chronologic age are a reflection of the severity of the sleep disorder. “Sleep efficiency,” defined as the percentage of the time spent in bed asleep is another index that can be used to determine the severity of the sleep disorder. Sleep efficiency is said to be abnormal when the percentage is below about 70%.

Sleep disorders and/or sleep disturbances include but are not limited to decreased quantity of REM sleep, increased undifferentiated sleep, immature organization of eye movements into discrete bursts during REM sleep, decreased time in bed, decreased total sleep time, decreased REM sleep latency, circadian rhythm disruption, and increased proportion of stage 1 sleep, or any combination thereof.

Sleep is increasingly recognized as important to public health, with sleep insufficiency linked to motor vehicle crashes, industrial disasters, and medical and other occupational errors. Unintentionally falling asleep, nodding off while driving, and having difficulty performing daily tasks because of sleepiness all may contribute to these hazardous outcomes. Persons experiencing sleep insufficiency are also more likely to suffer from chronic diseases such as hypertension, diabetes, depression, and obesity, as well as from cancer, increased mortality, and reduced quality of life and productivity. Sleep insufficiency may be caused by broad scale societal factors such as round-the-clock access to technology and work schedules, but sleep disorders such as insomnia or obstructive sleep apnea also play an important role. An estimated 50-70 million US adults have a sleep or wakefulness disorder.

A “normal” or “restful” sleep period is defined as a sleep period uninterrupted by wakefulness. Alternatively, a said period can be defined by the recommended or appropriate amount of sleep for the subject's age category, e.g., (i) infants 0-3 months=about 11 to about 19 hours; (ii) infants about 4 to about 11 months=about 12 to about 18 hours; (iii) toddlers about 1 to about 2 years=about 9 to about 16 hours; (iv) preschoolers about 3 to about 5 years=about 10 to about 14 hours; (v) school-aged children about 6 to about 13 years=about 7 to about 12 hours; (v) teenagers about 14 to about 17 years=about 7 to about 11 hours; (vi) young adults about 18 to about 25 years=about 6 to about 11 hours; (vii) adults about 26 to about 64 years=about 6 to about 10 hours; and (viii) older adults ≥65 years=about 5 to about 9 hours. Thus, for treating sleep disturbance in a subject, the treatment can result in a restful sleep period of at least about 4, about 5, about 6, about 7, about 8, about 9, about 10, about 11, or about 12 hours.

How much sleep is needed by a subject varies between individuals but generally changes with age. The National Institutes of Health suggests that school-age children need at least 10 hours of sleep daily, teens need 9-10 hours, and adults need 7-8 hours. According to data from the National Health Interview Survey, nearly 30% of adults reported an average of <6 hours of sleep per day in 2005-2007. Further, in 2009, only 31% of high school students reported getting at least 8 hours of sleep on an average school night. Similar recommendations are provided by the National Sleep Foundation (https://sleepfoundation.org/press-release/national-sleep-foundation-recommends-new-sleep-times/page/0/1):

TABLE 1

| | | May be | |
|---|---|---|---|
| Age | Recommended | appropriate | Not recommended |
| Newborns | 14 to 17 hours | 11 to 13 hours | Less than 11 hours |
| 0-3 months | | 18 to 19 hours | More than 19 hours |
| Infants | 12 to 15 hours | 10 to 11 hours | Less than 10 hours |
| 4-11 months | | 16 to 18 hours | More than 18 hours |
| Toddlers | 11 to 14 hours | 9 to 10 hours | Less than 9 hours |
| 1-2 years | | 15 to 16 hours | More than 16 hours |
| Preschoolers | 10 to 13 hours | 8 to 9 hours | Less than 8 hours |
| 3-5 years | | 14 hours | More than 14 hours |
| School-aged | 9 to 11 hours | 7 to 8 hours | Less than 7 hours |
| Children | | 12 hours | More than 12 hours |
| 6-13 years | | | |
| Teenagers | 8 to 10 hours | 7 hours | Less than 7 hours |
| 14-17 years | | 11 hours | More than 11 hours |
| Young Adults | 7 to 9 hours | 6 hours | Less than 6 hours |
| 18-25 years | | 10 to 11 hours | More than 11 hours |
| Adults | 7 to 9 hours | 6 hours | Less than 6 hours |
| 26-64 years | | 10 hours | More than 10 hours |
| Older Adults | 7 to 8 hours | 5 to 6 hours | Less than 5 hours |
| ≥65 years | | 9 hours | More than 9 hours |

There are several different scientifically acceptable ways to measure a sleep period uninterrupted by wakefulness. First, electrodes attached to the head of a subject can measure electrical activity in the brain by electroencephalography (EEG). This measure is used because the EEG signals associated with being awake are different from those found during sleep. Second, muscle activity can be measured using electromyography (EMG), because muscle tone also differs between wakefulness and sleep. Third, eye movements during sleep can be measured using electro-oculography (EOG). This is a very specific measurement that helps to identify Rapid Eye Movement or REM sleep. Any of these methods, or a combination thereof, can be used to determine if a subject obtains a restful sleep period following administration of at least one aminosterol or a salt or derivative thereof to the subject.

Further, circadian rhythm regulation can be monitored in a variety of ways, including but not limited to monitoring wrist skin temperature as described by Sarabia et al. 2008.

In some embodiments, administration of a therapeutically effective fixed dose of an aminosterol composition to an ASD patient with disturbed sleep results in improvement in frequency of normal or restful sleep as determined by a clinically recognized assessment scale for one or more types of sleep dysregulation, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

Example 1 describes several tools used to measure and evaluate the effect of amino sterol treatment on sleep, including for example:

(1) Sleep Diary (participants completed a sleep diary on a daily basis throughout the study. The diaries included time into bed and estimated time to sleep as well as wake time and duration during the night);

(2) I-Button Temperature Assessment. The I-Button is a small, rugged self-sufficient system that measures temperature and records the results in a protected memory section. The Thermochron I-Button DS1921H (Maxim Integrated, Dallas, Tex.) was used for skin temperature measurement. I-Buttons were programmed to sample every 10 mins., and attached to a double-sided cotton sport wrist band using Velcro, with the sensor face of the I-Button placed over the inside of the wrist, on the radial artery of the dominant hand. Subjects removed and replaced the data logger when necessary (i.e., to have a bath or shower). The value of skin temperature assessment in sleep research is that the endogenous skin warming resulting from increased skin blood flow is functionally linked to sleep propensity. From the collected data, the mesor, amplitude, acrophase (time of peak temperature), Rayleight test (an index of interdaily stability), mean waveforms are calculated);

(3) Unified Parkinson's Disease Rating Scale (UPDRS), sections 1.7 (sleep problems), 1.8 (daytime sleepiness) and 1.13 (fatigue);

(4) Parkinson's Disease Fatigue Scale (PFS-16);

(5) REM Sleep Behavior Disorder Screening Questionnaire; and

(6) Parkinson's Disease Sleep Scale.

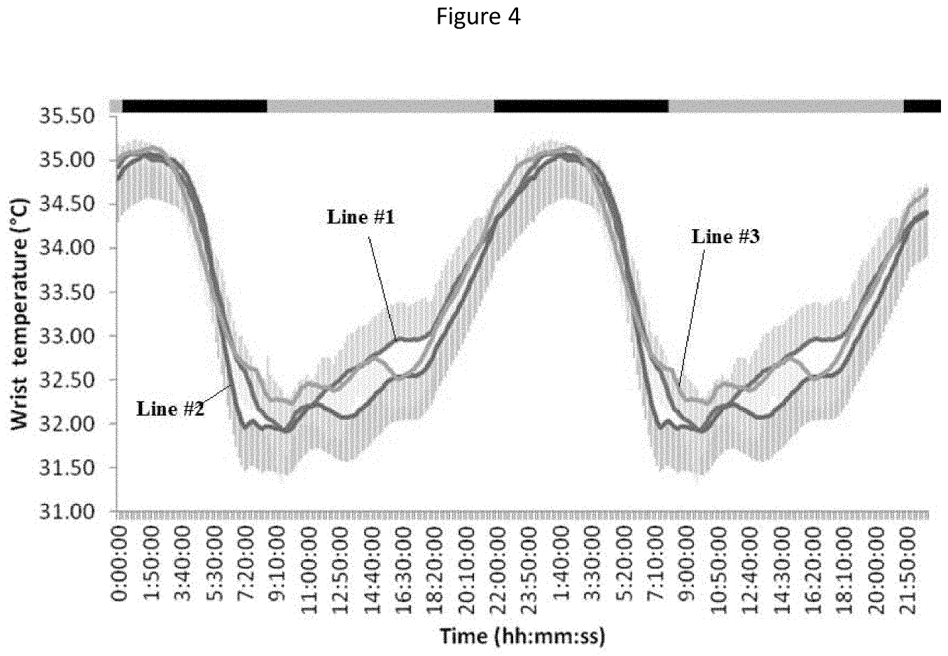

The data detailed in Example 1 described how circadian system status was evaluated by continuously monitoring wrist skin temperature (Thermochron iButton DS1921H; Maxim, Dallas) following published procedures (Sarabia et al. 2008). Further, an analysis was done with respect to the sleep data, the body temperature data, and fatigue data. The frequency of arm or leg thrashing reported in the sleep diary diminished progressively from 2.2 episodes/week at baseline to 0 at maximal dose (100% improvement). Total sleep time increased progressively from 7.1 hours at baseline to 8.4 hours at 250 mg (an 18% increase) and was consistently higher than baseline beyond 125 mg (FIG. 4). Unlike stool-related indices, the improvement in many CNS symptoms persisted during wash-out.

Circadian rhythm of skin temperature was evaluable in 12 patients (i.e., those who had recordings that extended from baseline through washout). Circadian system functionality was evaluated by continuously monitoring wrist skin temperature using a temperature sensor (Thermochron iButton DS1921H; Maxim, Dallas, Tex.) (Sarabia et al. 2008). Briefly, this analysis includes the following parameters: (i) the inter-daily stability (the constancy of 24-hour rhythmic pattern over days, IS); (ii) intra-daily variability (rhythm fragmentation, IV); (iii) average of 10-minute intervals for the 10 hours with the minimum temperature (L10); (iv) average of 10-minute intervals for the 5 hours with the maximum temperature (M5) and the relative amplitude (RA), which was determined by the difference between M5 and L10, divided by the sum of both. Finally, the Circadian Function Index (CFI) was calculated by integrating IS, IV, and RA. Consequently, CFI is a global measure that oscillates between 0 for the absence of circadian rhythmicity and 1 for a robust circadian rhythm.

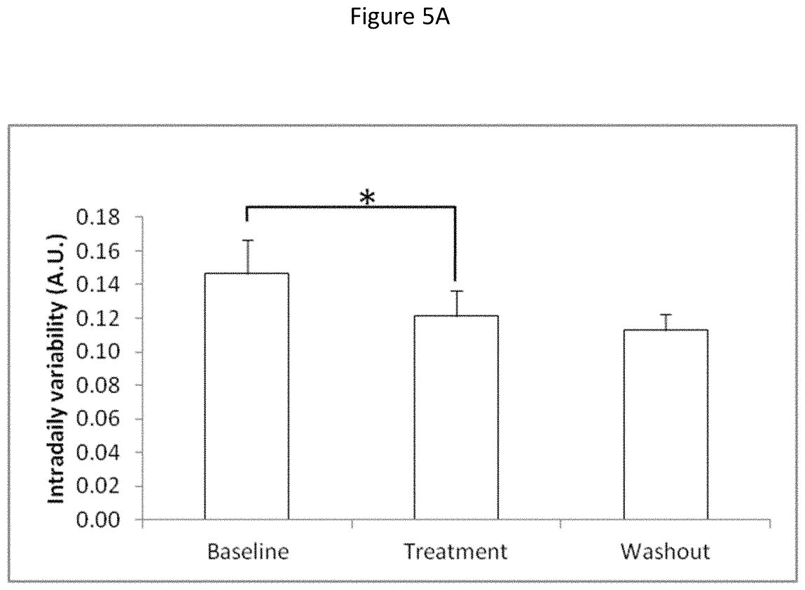

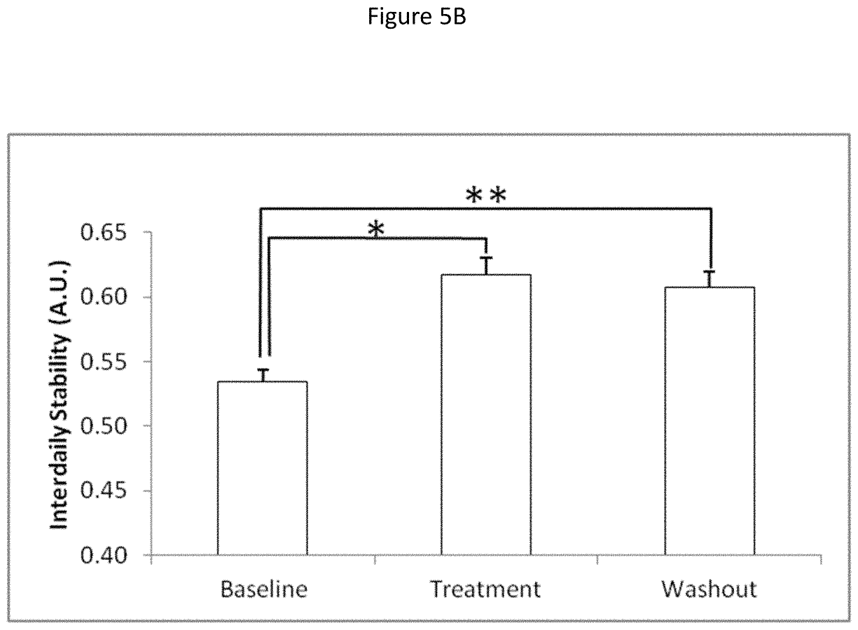

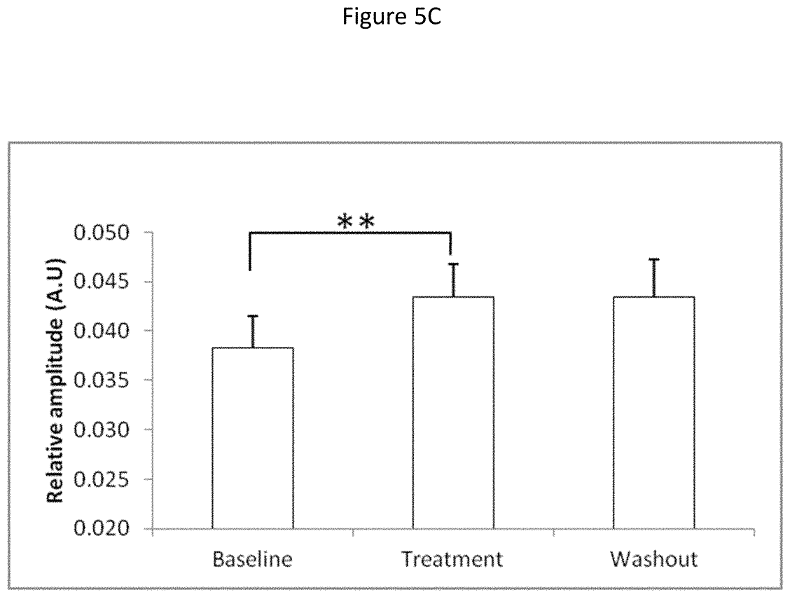

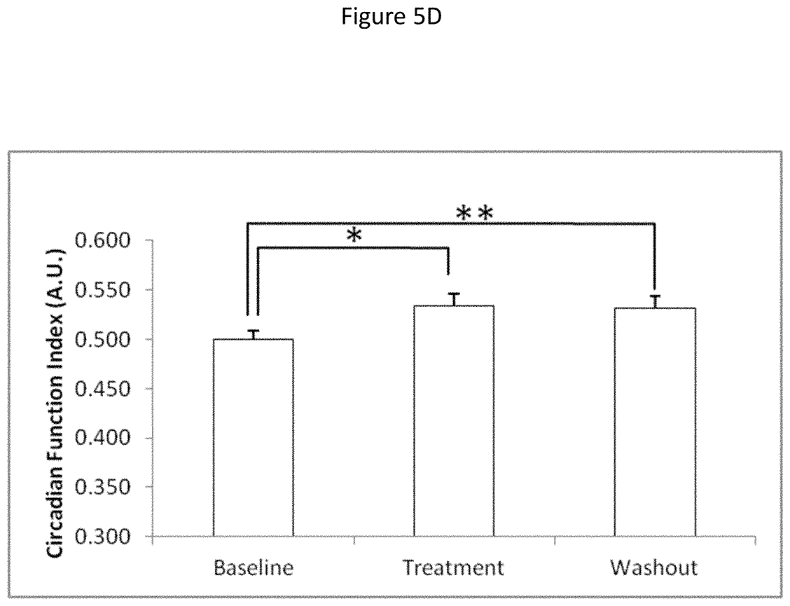

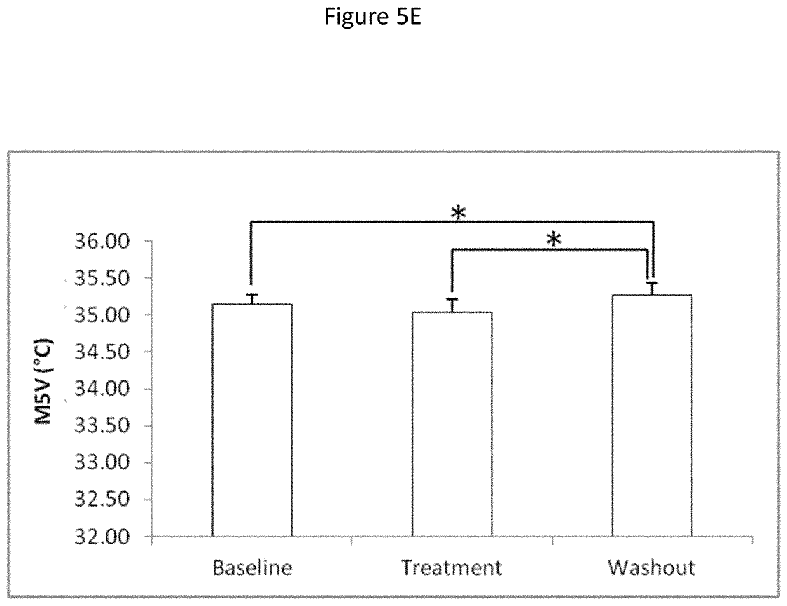

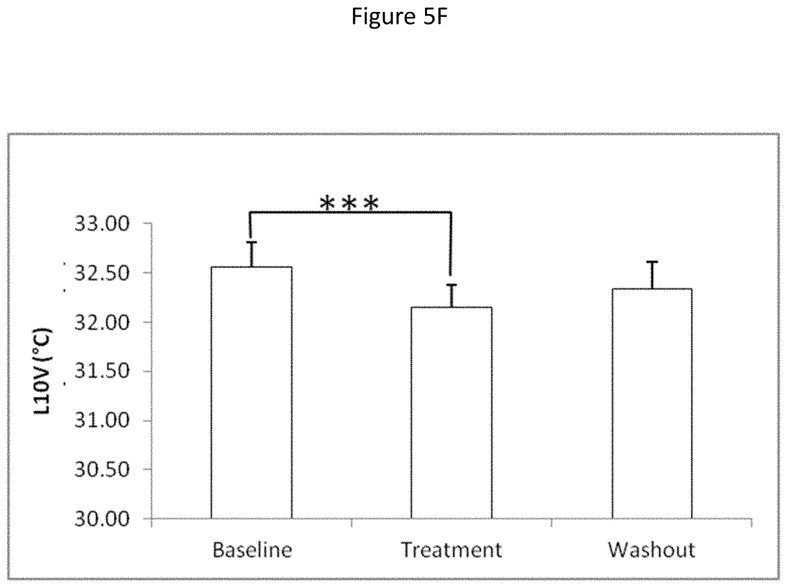

A comparison was performed of circadian rhythm parameters during the baseline, fixed dose and washout periods. Aminosterol administration improved all markers of healthy circadian function, including increasing rhythm stability, relative amplitude, and circadian function index, while reducing rhythm fragmentation. The improvement persisted for several of these circadian parameters during the wash-out period. (FIG. 5). Improvements were also seen in REM-behavior disorder (RBD) and sleep. RBD and total sleep time also improved progressively in a dose-dependent manner.

3. Cognitive Impairment

Another symptom associated with ASD is cognitive impairment. Cognitive impairment, including mild cognitive impairment (MCI), is characterized by increased memory or thinking problems exhibited by a subject as compared to a normal subject of the same age. Some degree of cognitive impairment is present in the majority of individuals with classical autism or synucleopathies like Parkinson's disease (PD).

Cognitive impairment may entail memory problems including a slight but noticeable and measurable decline in cognitive abilities, including memory and thinking skills. When MCI primarily affects memory, it is known as “amnestic MCI.” A person with amnestic MCI may forget information that would previously have been easily recalled, such as appointments, conversations, or recent events, for example. When MCI primarily affects thinking skills other than memory, it is known as “nonamnestic MCI.” A person with nonamnestic MCI may have a reduced ability to make sound decisions, judge the time or sequence of steps needed to complete a complex task, or with visual perception, for example.

Mild cognitive impairment is a clinical diagnosis. A combination of cognitive testing and information from a person in frequent contact with the subject is used to fully assess cognitive impairment. A medical workup includes one or more of an assessment by a physician of a subject's medical history (including current symptoms, previous illnesses, and family history), assessment of independent function and daily activities, assessment of mental status using brief tests to evaluate memory, planning, judgment, ability to understand visual information, and other key thinking skills, neurological examination to assess nerve and reflex function, movement, coordination, balance, and senses, evaluation of mood, brain imaging, or neuropsychological testing. Diagnostic guidelines for MCI have been developed by various groups, including the Alzheimer's Association partnered with the National Institute on Aging (NIA), an agency of the U.S. National Institutes of Health (NIH). Jack et al., 2011; McKhann et al., 2011; Albert et al., 2011. Recommendations for screening for cognitive impairment have been issued by the U.S. Preventive Services Task Force. Screening for Cognitive Impairment in Older Adults, U.S. Preventive Services Task Force (March 2014), https://www.uspreventiveservicestaskforce.org/Home/GetFileByID/1882. For example, the Mini Mental State Examination (MMSE) may be used. (Palsetia et al., 2018; Kirkevold, 0. & Selbaek, G., 2015). With the MMSE, a score of 24 or greater (out of 30) may indicate normal cognition, with lower scores indicating severe (less than or equal to 9 points), moderate (10-18 points), or mild (19-23 points) cognitive impairment. Other screening tools include the Informant Questionnaire on Cognitive Decline in the Elderly (IQCODE), in which an average score of 3 indicates no cognitive decline and a score greater than 3 indicates some decline. Jorm, A. F. 2004. Alternatively, the 7-Minute Screener, Abbreviated Mental Test Score (AMTS), Cambridge Cognitive Examination (CAMCOG), Clock Drawing Test (CDT), General Practitioner Assessment of Cognition (GPCOG), Mini-Cog, Memory Impairment Screen (MIS), Montreal Cognitive Assessment (MoCA), Rowland Universal Dementia Assessment (RUDA), Self-Administered Gerocognitive Examination (SAGE), Short and Sweet Screening Instrument (SAS-SI), Short Blessed Test (SBT), St. Louis Mental Status (SLUMS), Short Portable Mental Status Questionnaire (SPMSQ), Short Test of Mental Status (STMS), or Time and Change Test (T&C), among others, are frequently employed in clinical and research settings. Cordell et al. 2013. Numerous examinations may be used, as no single tool is recognized as the “gold standard,” and improvements in score on any standardized examination indicate successful treatment of cognitive impairment, whereas obtaining a score comparable to the non-impaired population indicates total recovery.

In some embodiments, administration of a therapeutically effective fixed dose of an aminosterol composition to an ASD subject in need results in improvement of cognitive impairment as determined by a clinically recognized assessment scale, by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

As detailed in Example 1, cognitive impairment and the improvement following aminosterol treatment were assessed using several tools (1) Mini Mental State Examination (MMSE); (2) Trail Making Test (TMT) Parts A and B; and (3) Unified Parkinson's Disease Rating Scale (UPDRS), sections 1.1 (cognitive impairment).

Assessments were made at baseline and at the end of the fixed dose and washout periods for Example 1, and an analysis was done with respect to the cognition symptoms. The results showed that the total UPDRS score was 64.4 at baseline, 60.6 at the end of the fixed dose period and 55.7 at the end of the wash-out period (a 13.5% improvement). Part 1 of the UPDRS (which includes section 1.1, cognitive impairment) had a mean baseline score of 11.6, a fixed aminosterol dose mean score of 10.6, and a wash-out mean score of 9.5, demonstrating an almost 20% improvement (UPDRS cognitive impairment is rated from 1=slight improvement to 4=severe impairment, so lower scores correlate with better cognitive function). In addition, MMSE improved from 28.4 at baseline to 28.7 during treatment and to 29.3 during wash-out (the MMSE has a total possible score of 30, with higher scores correlating with better cognitive function). Unlike stool-related indices, the improvement in many CNS symptoms persisted during wash-out.

4. Depression

Depression is another ASD symptom that can be used as a marker to determine effective aminosterol dosing according to the methods of the invention. Clinical depression can be associated with ASD and is characterized by a sad, blue mood that goes above and beyond normal sadness or grief. Major depression is an episode of sadness or apathy along with other symptoms that lasts at least two consecutive weeks and is severe enough to interrupt daily activities. Depressive events feature not only negative thoughts, moods, and behaviors but also specific changes in bodily functions (like, eating, sleeping, energy and sexual activity, as well as potentially developing aches or pains). Doctors clinically diagnose depression; there is no laboratory test or X-ray for depression.

Increasingly sophisticated forms of brain imaging, such as positron emission tomography (PET), single-photon emission computed tomography (SPECT), and functional magnetic resonance imaging (fMRI), permit a much closer look at the working brain than was possible in the past. An fMRI scan, for example, can track changes that take place when a region of the brain responds during various tasks. A PET or SPECT scan can map the brain by measuring the distribution and density of neurotransmitter receptors in certain areas. Use of this technology has led to a better understanding of which brain regions regulate mood and how other functions, such as memory, may be affected by depression. Areas that play a significant role in depression are the amygdala, the thalamus, and the hippocampus.

Research shows that the hippocampus is smaller in some depressed people. For example, in one fMRI study published in The Journal of Neuroscience, investigators studied 24 women who had a history of depression. On average, the hippocampus was 9% to 13% smaller in depressed women as compared with those who were not depressed. The more bouts of depression a woman had, the smaller the hippocampus. Stress, which plays a role in depression, may be a key factor, since experts believe stress can suppress the production of new neurons (nerve cells) in the hippocampus.

Researchers are exploring possible links between sluggish production of new neurons in the hippocampus and low moods. An interesting fact about antidepressants supports this theory. These medications immediately boost the concentration of chemical messengers in the brain (neurotransmitters). Yet people typically don't begin to feel better for several weeks or longer. Experts have long wondered why, if depression were primarily the result of low levels of neurotransmitters, people don't feel better as soon as levels of neurotransmitters increase. The answer may be that mood only improves as nerves grow and form new connections, a process that takes weeks. In fact, animal studies have shown that antidepressants do spur the growth and enhanced branching of nerve cells in the hippocampus. So, the theory holds, the real value of these medications may be in generating new neurons (a process called neurogenesis), strengthening nerve cell connections, and improving the exchange of information between nerve circuits.

Thus, in one embodiment of the invention, encompassed are methods of treating, preventing, and/or slowing the onset or progression of depression associated with ASD comprising administering therapeutically effective fixed dose of an amino sterol composition according to the invention to an ASD subject in need. While not wishing to be bound by theory, it is theorized that the aminosterol compositions of the invention trigger neurogenesis, which functions to combat depression.

In some embodiments, the methods of the invention produce an improvement in an ASD subject's clinical depression. An improvement in an ASD subject's depression can be measured using any clinically-recognized measurement. For example, improvement can be measured using a depression rating scale. In one embodiment of the invention, following treatment an ASD subject experiences an about 5, about 10, about 15, about 20, about 25, about 30, about 35, about 40, about 45, about 50, about 55, about 60, about 65, about 70, about 75, about 80, about 85, about 90, about 95 or an about 100% improvement. The improvement can be measured using any clinically recognized tool or assessment.

As detailed in Example 1, depression and/or mood and the improvement following aminosterol treatment in PD subjects were assessed using several tools:

(1) Beck Depression Inventory (BDI-II);

(2) Unified Parkinson's Disease Rating Scale (UPDRS), sections 1.3 (depressed mood), 1.4 (anxious mood), 1.5 (apathy), and 1.13 (fatigue); and

(3) Parkinson's Disease Fatigue Scale (PFS-16).

Assessments were made at baseline and at the end of the fixed dose and washout periods. An analysis was done with respect to depression and mood scores. Total UPDRS score was 64.4 at baseline, 60.6 at the end of the fixed dose period and 55.7 at the end of the wash-out period, demonstrating a 13.5% improvement, and Part 1 of the UPDRS (which includes mood and depression scores) went from a mean score of 11.6 at baseline, to a mean of 10.6 during the fixed aminosterol dose period, with a mean score of 9.5 during the washout period, demonstrating an improvement of 18%. In addition, BDI-II scores decreased from 10.9 at baseline to 9.9 during treatment and 8.7 at wash-out, showing an improvement in depression scoring of 20%. Unlike stool-related indices, the improvement in many CNS symptoms persisted during wash-out.

5. Alpha-Synuclein Aggregation

Alpha-synuclein is a potent pro-inflammatory hormone. Inflammation can be blocked by either of two strategies. First, inflammation can be blocked by reducing the tissue concentration of alpha-synuclein by decreasing or stopping production of alpha-synuclein. Alternatively, inflammation can be blocked by interrupting the signaling between alpha-synuclein and inflammatory cells that express CD11b. The subject of the methods of the invention can be any mammal, including a human. The inflammatory disease or condition caused by excessive expression of neuronal alpha synuclein can be a neurodegenerative disorder (NDD), such as ASD.

In some embodiments of the invention, ASD patient populations particularly susceptible to excessive production or secretion of alpha-synuclein can benefit from the methods of the invention and are targeted for therapy, including for example preventative therapy. For example, a patient population having a mutated form of alpha-synuclein resulting in increased amounts of alpha-synuclein in tissues can be treated using the methods of the invention.

The methods of the invention can result in a decrease in intensity of inflammation, blood levels of inflammatory markers, inflammatory markers in tissue, or number of inflammatory cells in tissue, or a combination thereof, as compared to a control or as compared to the qualitative or quantitative amount from the same PD patient or subject prior to treatment. For example, the decrease can be about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The improvement can be measured using any clinically recognized tool or assessment.

In some embodiments of the invention, ASD patient populations particularly susceptible to excessive production or secretion of alpha-synuclein can benefit from the methods of the invention and are targeted for therapy, including for example preventative therapy. For example, a patient population having a mutated form of alpha-synuclein resulting in increased amounts of alpha-synuclein in tissues can be treated using the methods of the invention. Another example of a patient population susceptible for high levels of alpha-synuclein are patients having chronic inflammatory conditions or diseases. A still further example is a patient population having elevated levels of alpha-synuclein aggregation in their enteric nerve cells, manifesting as a constipation.

Based on the data detailed in Example 1, it is believed that administration of an aminosterol reduces the formation of neurotoxic αS aggregates in vivo, and stimulates gastrointestinal motility in patients with neurodiseases such as ASD and constipation. The observation that the dose required to achieve a prokinetic response increases with constipation severity supports the hypothesis that the greater the burden of αS impeding neuronal function, the higher the dose of aminosterol required to restore normal bowel function as well as address other symptoms of alpha-synuclein aggregation. The data detailed in Example 1 is the first proof of concept demonstration that directly targeting αS pharmacologically can achieve beneficial GI, autonomic and CNS responses.

This data in Example 1 supports the hypothesis that gastrointestinal dysmotility in neurodiseases such as ASD results from the progressive accumulation of αS in the ENS, and that aminosterols can restore neuronal function by displacing αS and stimulating enteric neurons. Improvements were also seen in cognitive function (MMSE scores), hallucinations, REM-behavior disorder (RBD) and sleep. These improvements are unlikely to be due to improved gastric motility and increased absorption of dopaminergic medications, since improvement persisted during the 2-week wash-out period, i.e., in the absence of study drug, thus indicating the likely improvement based upon aminosterol treatment restoring neuronal function by displacing αS and stimulating enteric neurons. These results demonstrate that the ENS in neurodisease such as PD is not irreversibly damaged and can be restored to normal function using the methods of the invention.

6. Avoidance of Eye Contact

Individuals with autism spectrum disorder (ASD) often report that looking in the eyes of others is uncomfortable for them or that it is terribly stressful. One of the earliest recognizable symptoms in young developing children noticed as an indicator of ASD is the avoidance of eye contact by the subject with ASD. The subcortical pathway, which consists of superior colliculus, pulvinar nucleus of the thalamus, and amygdala, enables rapid and automatic face processing. A specific component of this pathway—i.e., the amygdala—has been shown to be abnormally activated in paradigms where individuals had to specifically attend to the eye-region. Functional MRI, has been used to investigate the effect of constraining gaze in the eye-region during dynamic emotional face perception in groups of participants with ASD and typical controls. Differences in activation in the subcortical face processing system (superior colliculus, pulvinar nucleus of the thalamus and amygdala) were computed for the same stimuli seen freely or with the gaze constrained in the eye-region. When constrained to look in the eyes, individuals with ASD show abnormally high activation in the subcortical system, which may be at the basis of their eye avoidance in daily life (Hadjikhani et al., 2017).

In some embodiments of the method described herein, the ASD symptom to be evaluated is a avoidance of eye contact, wherein (a) the method results in a positive change in the amount of eye contact engaged in by the subject; (b) the method results in a positive change in the amount of eye contact engaged in by the subject, wherein the positive change is defined as (i) an increase in the amount of time of eye contact of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of instances in which the subject avoids eye contact selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. A positive change in the amount of eye contact may refer to (a) number of instances of engagement in eye-contact by the subject increasing, or (b) amount of time that subject is able to sustain eye-contact once engaged increasing.

7. Echolalia and/or Palilalia

Echolalia is meaningless repetition of another person's spoken words, and palilalia is a meaningless repetition of the same person's words. It is estimated that up to 75% of persons on the autism spectrum have exhibited echolalia and/or palilalia. A symptom of some children with ASD is the struggle to produce spontaneous speech. Studies have shown that in some cases echolalia is used as a coping mechanism allowing a person with autism to contribute to a conversation when unable to produce spontaneous speech (Volkmar et al., 2005).

In some embodiments of the method herein, the ASD symptom to be evaluated is echolalia and/or palilalia wherein (a) the method results in a decreased number of instances in which the subject engages in echolalia and/or palilalia over a defined period of time; (b) the method results in a decreased number of instances in which the subject engages in echolalia and/or palilalia over a defined period of time and the decrease is defined as a reduction in instances of engagement in echolalia and/or palilalia selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject ceasing to engage in echolalia and/or palilalia. An instance may comprise the utterance of a single word or a phrase.

8. Self-Injury

“Self-injury” refers to purposeful injury to the self, by a subject with ASD. About one in four children in the U.S. with autism hit, scratch or otherwise hurt themselves (Soke et al., 2016). Children who engage in self-injury tend to have mood and behavioral challenges, as well as cognitive impairment. Self-injury may be accomplished by hitting, scratching, or biting one's self. The release of endorphins upon self-injury has been postulated as a reason for why ASD subjects may self-injure, seeking the anesthetic and calming effects. Self-injurious behavior may also be the result of involuntary movement associated with seizures.

In some embodiments of the method described herein, the ASD symptom to be evaluated is self-injury wherein (a) the method results in a decreased number of instances in which the subject self-injures over a defined period of time; (b) the method results in a decreased number of instances in which the subject self-injures over a defined period of time selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject ceasing to self-injure.

In some embodiments, (a) the method results in a decreased severity of self-injuries over a defined period of time, wherein the decreased severity is measured using a medically recognized technique; (b) the method results in a decreased severity of self-injuries over a defined period of time, wherein the decrease in severity is selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject ceasing to self-injure; wherein the decreased severity is measured using a medically recognized technique. The medically recognized technique may be one or more techniques selected from the group consisting of Barell Matrix, The Abbreviated Injury Scale, and the Glasgow Coma Scale.

9. Repetitive Motion

DSM-5 specifies repetitive motion or repetitive and restricted behaviors (RRBs) as one of two domains required for the diagnosis of an autism spectrum disorder. Repetitive-motion is among the first signs of autism to emerge in toddlers. Non-limiting examples of repetitive-motion include hand-flapping, rocking the body back and forth, foot or hand tapping, teeth grinding, and nail biting.

In some embodiments of the method described herein, the ASD symptom to be evaluated is repetitive motion wherein (a) the method results in a decreased number of instances in which the subject engages in repetitive motion over a defined period of time; (b) the method results in a decreased number of instances in which the subject engages in repetitive motion over a defined time period, wherein the decrease in number is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject ceasing to engage in repetitive motion.

In some embodiments, (a) the method results in a decreased severity of repetitive motion over a defined period of time; (b) the method results in a decreased severity of repetitive motion over a defined period of time, wherein the decrease in severity is by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (c) the method results in the subject ceasing to engage in repetitive motion; wherein the decrease in severity comprises a decrease in number of repetitions per minute (RPM) for the repetitive motion.

### V. Definitions

The following definitions are provided to facilitate understanding of certain terms used throughout this specification.

Technical and scientific terms used herein have the meanings commonly understood by one of ordinary skill in the art, unless otherwise defined. Any suitable materials and/or methodologies known to those of ordinary skill in the art can be utilized in carrying out the methods described herein.

As used in the description of the invention and the appended claims, the singular forms “a”, “an” and “the” are used interchangeably and intended to include the plural forms as well and fall within each meaning, unless the context clearly indicates otherwise. Also, as used herein, “and/or” refers to and encompasses any and all possible combinations of one or more of the listed items, as well as the lack of combinations when interpreted in the alternative (“or”).

As used herein the term “amino sterol” refers to an amino derivative of a sterol. Non-limiting examples of suitable aminosterols for use in the composition and methods disclosed herein are Aminosterol 1436, squalamine, aminosterols isolated from Squalus acanthias, and isomers, salts, and derivatives each thereof.

The term “administering” as used herein includes prescribing for administration as well as actually administering, and includes physically administering by the subject being treated or by another.

As used herein “subject,” “patient,” or “individual” refers to any subject, patient, or individual, and the terms are used interchangeably herein. In this regard, the terms “subject,” “patient,” and “individual” includes mammals, and, in particular humans. When used in conjunction with “in need thereof,” the term “subject,” “patient,” or “individual” intends any subject, patient, or individual having or at risk for a specified symptom or disorder.

As used herein, the phrase “therapeutically effective” or “effective” in context of a “dose” or “amount” means a dose or amount that provides the specific pharmacological effect for which the compound or compounds are being administered. It is emphasized that a therapeutically effective amount will not always be effective in achieving the intended effect in a given subject, even though such dose is deemed to be a therapeutically effective amount by those of skill in the art. For convenience only, exemplary dosages are provided herein. Those skilled in the art can adjust such amounts in accordance with the methods disclosed herein to treat a specific subject suffering from a specified symptom or disorder. The therapeutically effective amount may vary based on the route of administration and dosage form.

The terms “treatment,” “treating,” or any variation thereof includes reducing, ameliorating, or eliminating (i) one or more specified symptoms and/or (ii) one or more symptoms or effects of a specified disorder. The terms “prevention,” “preventing,” or any variation thereof includes reducing, ameliorating, or eliminating the risk of developing (i) one or more specified symptoms and/or (ii) one or more symptoms or effects of a specified disorder.

### EXAMPLES

### Example 1

This example describes an exemplary method of treating and/or preventing symptoms of Parkinson's disease (PD) in a clinical trial setting. The methods used in Example 1 to determine the dose of aminosterol may be used to determine the aminosterol dose in subsequent examples relating to ASD or symptoms of ASD.

Overview: The subjects of the trial all had PD and experienced constipation, which is a characteristic of PD. The primary objectives of the trial involving patients with PD and constipation were to evaluate the safety and pharmacokinetics of oral squalamine (ENT-01) and to identify the dose required to improve bowel function, which was used as a clinical endpoint.

Several non-constipation PD symptoms were also assessed as endpoints, including, for example, (1) sleep problems, including daytime sleepiness; (2) non-motor symptoms, such as (i) depression (including apathy, anxious mood, as well as depression), (ii) cognitive impairment (e.g., using trail making test and the UPDRS), (iii) hallucinations (e.g., using The University of Miami Parkinson's Disease Hallucinations Questionnaire (UM-PDHQ) and the UPDRS, (iv) dopamine dysregulation syndrome (UPDRS), (v) pain and other sensations, (vi) urinary problems, (vii) light headedness on standing, and (viii) fatigue (e.g., using Parkinson's Disease Fatigue Scale 9PFS-1t and the UPDRS); (3) motor aspects of experiences of daily living, such as (i) speech, (ii) saliva and drooling, (iii) chewing and swallowing, (iv) eating tasks, (v) dressing, (vi) hygiene, (vii) handwriting; (viii) doing hobbies and other activities, (ix) turning in bed, (x) tremor, (xi) getting out of bed, a car, or a deep chair, (xii) walking and balance, (xiii) freezing; (4) motor examination, such as (i) speech, (ii) facial expression, (iii) rigidity, (ix) finger tapping, (v) hand movements, (vi) pronation-supination movements of hands, (vii) toe tapping, (viii) leg agility, arising from chair, (ix) gait, (x) freezing of gait, (xi) postural stability, (xii) posture, (xiii) global spontaneity of movement (body bradykinesia), (xiv) postural tremor of the hands, (xv) kinetic tremor of the hands, (xvi) rest tremor amplitude, (xvii) constancy of rest tremor; (5) motor complications, such as (i) time spent with dyskinesias, (ii) functional impact of dyskinesias, (iii) time spent in the off state, (iv) functional impact of fluctuations, (v) complexity of motor fluctuations, and (vi) painful off-state dystonia.

Active Agent & Dosing:

Squalamine (ENT-01; Enterin, Inc.) was formulated for oral administration in the trial. The active ion of ENT-01, squalamine, an amino sterol originally isolated from the dogfish shark, has been shown to reverse gastrointestinal dysmotility in several mouse models of PD. In addition, ENT-01 has been shown to inhibit the formation of aggregates of αS both in vitro, and in a C. elegans model of PD in vivo (Perni et al., 2017). In the C. elegans model, squalamine produced a complete reversal of muscle paralysis.

ENT-01 is the phosphate salt of squalamine. For this study it has been formulated as a small 25 mg coated tablet. Dosing ranged from 25 mg to 250 mg, with dosages greater than 25 mg requiring multiple pills (e.g., 50 mg=two 25 mg pills). Dosing instructions=take 60 mins before breakfast with 8 oz. water. The dose was taken by each patient upon awakening on an empty stomach along with 8 oz. of water simultaneously to dopamine. The subject was not allowed to ingest any food for at least 60 minutes after study medication. The compound is highly charged and will adsorb to foodstuffs, so it was administered prior to feeding.

The phosphate salt of squalamine (ENT-01) is weakly soluble in water at neutral pH but readily dissolves at pH<3.5 (the pH of gastric fluid). Squalamine, as the highly water soluble dilactate salt has been extensively studied in over three Phase 1 and eight Phase 2 human clinical trials as an intravenous agent for the treatment of cancer and diabetic retinopathy. The compound is well tolerated in single and repeat intravenous administration, alone or in combination with other agents, to doses of at least 300 mg/m²).

In the current clinical trial, squalamine (ENT-01) was administered orally to subjects with PD who have long standing constipation. Although this trial was the first in man oral dosing study of ENT-01, humans have long been exposed to low doses of squalamine (milligram to microgram) in the various commercial dogfish shark liver extracts available as nutraceuticals (e.g., Squalamax). In addition, following systemic administration squalamine is cleared by the liver and excreted as the intact molecule (in mice) into the duodenum through the biliary tract. Drug related GI toxicology has not been reported in published clinical trials involving systemic administration of squalamine.

Squalamine (ENT-01) has limited bioavailability in rats and dogs. Based on measurement of portal blood concentrations following oral dosing of radioactive ENT-01 to rat's absorption of ENT-01 from the intestine is low. As a consequence, the principal focus of safety is on local effects on the gastrointestinal tract. However, squalamine (ENT-01) appears to be well tolerated in both rats and dogs.

The starting dose in the Stage 1 segment of the trial was 25 mg (0.33 mg/kg for a 75 kg subject). The maximum single dose in Stage 1 was 200 mg (2.7 mg/kg for a 75 kg subject). The maximum dose evaluated in Stage 2 of the trial was 250 mg/day (3.3 mg/kg/day for a 75 kg subject), and the total daily dosing exposure lasted no longer than 25 days.

The daily dosing range in the clinical trial was from 25 mg (14.7 mg/m²) to 250 mg (147 mg/m²). Oral dosing of squalamine (ENT-01), because of its low oral bioavailability, is not anticipated to reach significant plasma concentrations in human subjects. In preclinical studies, squalamine (ENT-01) exhibited an oral bioavailability of about 0.1% in both rats and dogs. In Stage 1 of this phase 2 study, oral dosing up to 200 mg (114 mg/m²) yielded an approximate oral bioavailability of about 0.1%, based on a comparison of a pharmacokinetic data of the oral dosing and the pharmacokinetic data measured during prior phase 1 studies of IV administration of squalamine.

Study Protocol:

The multicenter Phase 2 trial was conducted in two Stages: a dose-escalation toxicity study in Stage 1 and a dose range-seeking and proof of efficacy study in Stage 2.

PD symptoms were assessed using a number of different tools:

(1) Numeric Rating Scales for Pain and Swelling (scale of 0-10, with 0=no pain and 10=worst pain ever experienced);

(2) Rome-IV Criteria for Constipation (7 criteria, with constipation diagnosis requiring two or more of the following: (i) straining during at least 25% of defecations, (ii) lumpy or hard stools in at least 25% of defecations, (iii) sensation of incomplete evacuation for at least 25% of defecations, (iv) sensation of anorectal obstruction/blockage for at least 25% of defecations; (v) manual maneuvers to facilitate at least 25% of defecations; (vi) fewer than 3 defecations per week; and (vii) loose stools are rarely present without the use of laxatives;

(3) Constipation—Ease of Evacuation Scale (from 1-7, with 7=incontinent, 4=normal, and 1=manual disimpaction);

(4) Bristol Stool Chart, which is a patient-friendly means of categorizing stool characteristics (assessment of stool consistency is a validated surrogate of intestinal motility) and Stool Diary;

(5) Sleep Diary (participants completed a sleep diary on a daily basis throughout the study. The diaries included time into bed and estimated time to sleep as well as wake time and duration during the night);

(6) I-Button Temperature Assessment. The I-Button is a small, rugged self-sufficient system that measures temperature and records the results in a protected memory section. The Thermochron I-Button DS1921H (Maxim Integrated, Dallas, Tex.) was used for skin temperature measurement. I-Buttons were programmed to sample every 10 mins., and attached to a double-sided cotton sport wrist band using Velcro, with the sensor face of the I-Button placed over the inside of the wrist, on the radial artery of the dominant hand. Subjects removed and replaced the data logger when necessary (i.e., to have a bath or shower). The value of skin temperature assessment in sleep research is that the endogenous skin warming resulting from increased skin blood flow is functionally linked to sleep propensity. From the collected data, the mesor, amplitude, acrophase (time of peak temperature), Rayleight test (an index of interdaily stability), mean waveforms are calculated);

(7) Non-motor Symptoms Questionnaire (NMSQ);

(8) Beck Depression Inventory (BDI-II);

(9) Unified Parkinson's Disease Rating Scale (UPDRS), which consists of 42 items in four subscales (Part I=Non-Motor Aspects of Experiences of Daily Living (nM-EDL) (1.1 cognitive impairment, 1.2 hallucinations and psychosis, 1.3 depressed mood, Part II=Motor Aspects of Experiences of Daily Living (M-EDL), Part III=Motor Examination, and Part IV=Motor Complications;

(10) Mini Mental State Examination (MMSE);

(11) Trail Making Test (TMT) Parts A and B;

(12) The University of Miami Parkinson's Disease Hallucinations Questionnaire (UM-PDHQ);

(13) Parkinson's Disease Fatigue Scale (PFS-16);

(14) Patient Assessment of Constipation Symptoms (PAC-SYM);

(15) Patient Assessment of Constipation Quality of Life (PAC-QOL);

(16) REM Sleep Behavior Disorder Screening Questionnaire; and

(17) Parkinson's Disease Sleep Scale.

Exploratory end-points, in addition to constipation, included for example, (i) depression assessed using the Beck Depression Inventory (BDI-II) (Steer et al. 2000) and Unified Parkinson's Disease Rating Scale (UPDRS); (ii) cognition assessed using the Mini Mental State Examination (MMSE) (Palsteia et al., 2018), Unified Parkinson's Disease Rating Scale (UPDRS), and Trail Making Test (TMT); (iii) sleep and REM-behavior disorder (RBD) using a daily sleep diary, I-Button Temperature Assessment, a REM sleep behavior disorder (RBD) questionnaire (RBDQ) (Stiasny-Kolster et al. 2007), and the UPDRS; (iv) hallucinations assessed using the PD hallucinations questionnaire (PDHQ) (Papapetropoulos et al. 2008), the UPDRS, and direct questioning; (v) fatigue using the Parkinson's Disease Fatigue Scale (PFS-16) and the UPDRS; (vi) motor functions using the UPDRS; and (vii) non-motor functions using the UPDRS.

Assessments were made at baseline and at the end of the fixed dose and washout periods. Circadian system status was evaluated by continuously monitoring wrist skin temperature (Thermochron iButton DS1921H; Maxim, Dallas) following published procedures (Sarabia et al. 2008).

Based on these data, it is believed that administration of squalamine (ENT-01), a compound that can displace αS from membranes in vitro, reduces the formation of neurotoxic αS aggregates in vivo, and stimulates gastrointestinal motility in patients with PD and constipation. The observation that the dose required to achieve a prokinetic response increases with constipation severity supports the hypothesis that the greater the burden of αS impeding neuronal function, the higher the dose of squalamine (ENT-01) required to restore normal bowel function.

Study Design:

A multicenter Phase 2 trial was conducted in two Stages: a dose-escalation toxicity study in Stage 1 and a dose range-seeking and proof of efficacy study in Stage 2. The protocol was reviewed and approved by the institutional review board for each participating center and patients provided written informed consent.

Following successful screening, all subjects underwent a 14-day run-in period where the degree of constipation was assessed through a validated daily log (Zinsmeister et al., 2013) establishing baseline CSBMs/week. Subjects with an average of <3 CSBMs/week proceeded to dosing.

In Stage 1, ten (10) PD patients received a single escalating dose of squalamine (ENT-01) every 3-7 days beginning at 25 mg and continuing up to 200 mg or the limit of tolerability, followed by 2-weeks of wash-out. Duration of this part of the trial was 22-57 days. The 10 subjects in the sentinel group were assigned to Cohort 1 and participated in 8 single dosing periods. Tolerability limits included diarrhea or vomiting. A given dose was considered efficacious in stimulating bowel function (prokinetic) if the patient had a complete spontaneous bowel movement (CSBM) within 24 hours of dosing.

Each dose period was staggered, so that subjects 1-2 were administered a single dose of the drug at the lowest dose of 25 mg. Once 24 hours have elapsed, and provided there are no safety concerns, the patient was sent home and brought back on day 4-8 for the next dose. During the days the subjects are home, they completed the daily diaries and e-mailed them to the study coordinators. Subjects 3-10 were dosed after the first 2 subjects have been observed for 72 hours, i.e. on Day 4. Subjects 1-2 were also brought back on Day 4-8 and given a single dose of 50 mg. Once another 24 hours have elapsed and provided there are no safety concerns, the patients were all sent home and instructed to return on Day 7 for the next dosing level. This single dosing regimen was continued until each subject was given a single dose of 200 mg or has reached a dose limiting toxicity (DLT). DLT was the dose which induces repeated vomiting, diarrhea, abdominal pain or symptomatic postural hypotension within 24 hours of dosing.

In Stage 2, 34 patients were evaluated. First, 15 new PD patients were administered squalamine (ENT-01) daily, beginning at 75 mg, escalating every 3 days by 25 mg to a dose that had a clear prokinetic effect (CSBM within 24 hours of dosing on at least 2 of 3 days at a given dose), or the maximum dose of 175 mg or the tolerability limit. This dose was then maintained (“fixed dose”) for an additional 3-5 days. After the “fixed dose”, these patients were randomly assigned to either continued treatment at that dose or to a matching placebo, for an additional 4-6 days prior to a 2-week wash-out.

A second cohort of 19 patients received squalamine (ENT-01) escalating from 100 mg/day to a maximum of 250 mg/day without subsequent randomization to squalamine (ENT-01) or placebo. Criteria for dose selection and efficacy were identical to those used in the previous cohort.

Patient Population:

Patients were between 18 and 86 years of age and diagnosed with PD by a clinician trained in movement disorders following the UK Parkinson's Disease Society Brain Bank criteria (Fahn et al., 1987). Patients were required to have a history of constipation as defined by <3 CSBMs/week and satisfy the Rome IV criteria for functional constipation (Mearin et al., 2016) at screening, which requires 2 or more of the following: Straining during at least 25% of defecations; lumpy or hard stools in at least 25% of defecations; sensation of incomplete evacuation in at least 25% of defecations; sensation of anorectal obstruction/blockage in at least 25% of defecations; and/or manual maneuvers to facilitate at least 25% of defecations.

Baseline characteristics of patients are shown in Table 2. Patients in Stage 2 had somewhat longer duration of Parkinson's disease and higher UPDRS scores than participants in Stage 1.

TABLE 2
Baseline Characteristics of Dosed Patients

| | | | |
|---|---|---|---|
| | Stage 1** | Stage 2*** | Total |
| Characteristic | (n = 10) | (n = 34) | (n = 44) |

Sex-no. (%)

| | | | |
|---|---|---|---|
| Male | 5 (50) | 25 (73.5) | 30 (68.1) |
| Female | 5 (50) | 9 (26.5) | 14 (31.8) |
| White race-no. (%) | 8 (80) | 34 (100) | 42 (95.54) |

Age-yr

| | | | |
|---|---|---|---|
| Mean | 65.0 | 74.5 | 72.5 |
| Range | 58-70.5 | 60.6-84.2 | 58-84.2 |

Age at PD diagnosis-yr

| | | | |
|---|---|---|---|
| Mean | 61.1 | 67.7 | 66.2 |
| Range | 54.2-69 | 50.6-82.5 | 50.6-82.5 |

Duration of PD-yr

| | | | |
|---|---|---|---|
| Mean | 4.2 | 6.8 | 6.2 |
| Range | 1-11 | 0.3-17.3 | 0.3-17.3 |

Duration of constipation-yr

| | | | |
|---|---|---|---|
| Mean | 25.8 | 16.8 | 18.9 |
| Range | 1-65 | 0.5-66.0 | 0.5-66.0 |

UPDRS score

| | | | |
|---|---|---|---|
| Mean | 53.4 | 63.2 | 61.3 |
| Range | 33-88 | 24-122 | 24.0-122.0 |

Hoehn and Yahr-Stage

| Mean | 2.0 | 2.4 | 2.3 |
|---|---|---|---|
| Range | 2.0 | 1.0-5.0 | 1.0-5.0 |
| Constipation severity* - CSBM/wk- no. (%) | | | |
| 0-1 | 8(80) | 14(41.2) | 22 (50) |
| 1.1-2 | 2 (20) | 17(50) | 19 (43.2) |
| 2.1-3 | 0 | 3 (8.8) | 3 (6.8) |

*At baseline. Baseline value is the average number of CSBMs per week calculated at the end of the 2-week run-in period.
**In Stage 1, 10 patients received single escalating doses every 3-7 days starting at 25 mg and escalating up to dose limiting toxicity (DLT) or 200 mg, whichever came first, followed by a 2-week wash-out period.
***In Stage 2, 15 patients received daily doses starting at 75 mg and escalating every 3 days up to prokinetic dose (dose producing CSBMs on at least 2 of 3 days) or 175 mg, whichever came first, followed by an additional 2-4 days at that dose (“fixed dose” period) and were then randomized to treatment at the “fixed-dose” or placebo for 4-6 days. Wash-out lasted 2 weeks. The remaining 19 patients were escalated from 100 mg to prokinetic dose or 250 mg, whichever came first, followed by an additional 2-4 days at that dose and then a 2-week wash-out period.

Safety and Adverse Event (AE) Profile:

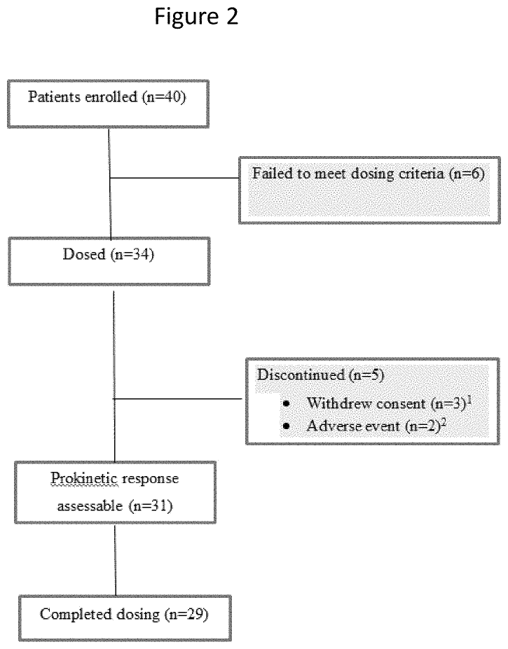

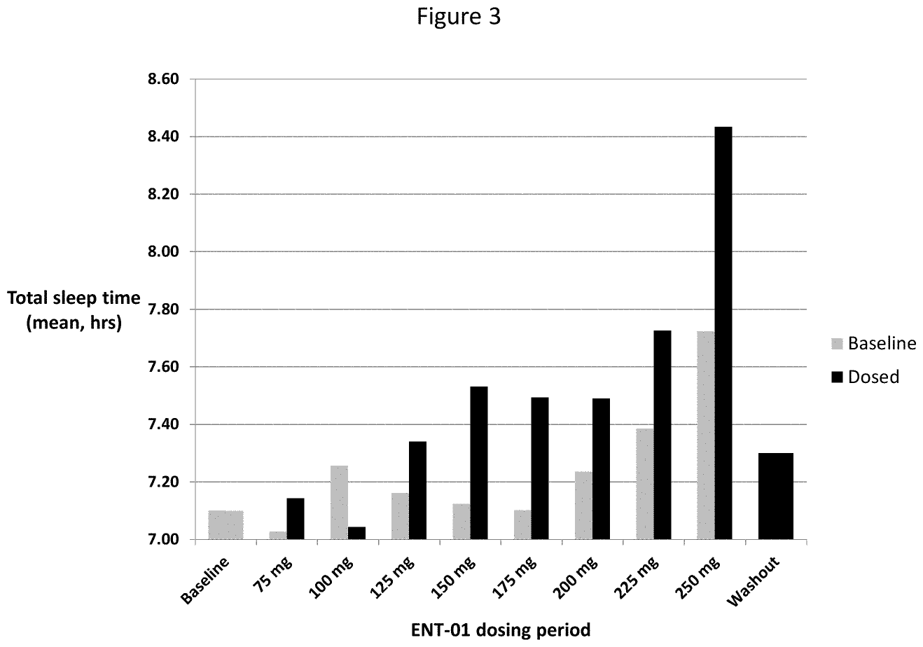

Fifty patients were enrolled and 44 were dosed. In Stage 1, 10 patients were dosed, 1 (10%) withdrew prior to completion and 9 (90%) completed dosing. In stage 2, 6 (15%) patients had ≥3 CSBM/week at the end of the run-in period and were excluded, 34 patients were dosed and bowel response was assessable in 31 (91%). Two patients (5.8%) were terminated prior to completion because of recurrent dizziness, and 3 others withdrew during dosing (8.8%): 2 because of diarrhea and 1 because of holiday. Fifteen patients were randomized. Study-drug assignments and patient disposition are shown in Table 3 and FIG. 2.

TABLE 3
Study drug assignments and adherence to treatment

| | Stage 1 | Stage 2 |
|---|---|---|
| Enrolled | 10 | 40 |
| Failed prior to dosing | 0 | 6 |
| Dosed | 10 | 34 |
| 25-200 mg | 10 | |
| 75-175 mg | | 19 |
| 100-250 mg | | 15 |
| Terminated (%) | 0 (0) | 2* (5.8) |
| Withdrew (%) | 1 (10) | 3 (8.8) |
| Completed dosing (%) | 9 (90) | 31** (91) |
| Randomized | | 15 |
| Treatment | | 6 |
| Placebo | | 9 |

The 2 patients who were terminated **29 patients completed dosing but an additional 2 who withdrew had an assessable prokinetic end-point.

Most AEs were confined to the GI tract (88% in Stage 1 and 63% in Stage 2). The most common AE was nausea which occurred in 4/10 (40%) patients in Stage 1 and in 18/34 (52.9%) in Stage 2 (Table 2). Diarrhea occurred in 4/10 (40%) patients in Stage 1 and 15/34 (44%) in Stage 2. One patient withdrew because of recurrent diarrhea. Other GI related AEs included abdominal pain 11/44 (32%), flatulence 3/44 (6.8%), vomiting 3/44 (6.8%), worsening of acid reflux 2/44 (4.5%), and worsening of hemorrhoids 1/44 (2.2%). One patient had a lower GI bleed (Serious adverse event, SAE) during the withdrawal period. This patient was receiving aspirin, naproxen and clopidogrel at the time of the bleed, and colonoscopy revealed large areas of diverticulosis and polyps. This SAE was considered unrelated to study medication. The only other noteworthy AE was dizziness 8/44 (18%). Dizziness was graded as moderate in one patient who was receiving an alpha-adrenergic blocking agent (Terazosin). This patient was withdrawn from the study and recovered spontaneously. All other AEs resolved spontaneously without discontinuation of squalamine (ENT-01). The relationship between dose and AEs is shown in Table 4.

TABLE 4
All adverse events (n, %)

| Enrolled | Stage 1 (n = 10) | Stage 2 (n = 40) |
|---|---|---|
| Dosed | 10 | 34 |
| GI: | | |
| Nausea | | |
| Mild | 4(40) | 18(52) |
| Moderate | 0 | 1(2.9) |
| Diarrhea | | |
| Mild | 1(10) | 12(35) |
| Moderate | 3(30) | 2(5.8) |
| Severe | 0 | 1(2.9) |
| Vomiting | | |
| Mild | 1(10) | 2(5.8) |
| Moderate | 0 | 0 |
| Abdominal pain | | |
| Mild | 2(20) | 4(11.7) |
| Moderate | 3(30) | 2(5.8) |
| Flatulence | | |
| Mild | 2(20) | 1(3) |
| Moderate | 0 | 0 |
| Loss of appetite* | | |
| Mild | 1(10) | 0 |
| Moderate | 0 | 0 |
| Worsening acid reflux | | |
| Mild | 0 | 4(11.7) |
| Moderate | 0 | 0 |
| Worsening hemorrhoid | | |
| Mild | 0 | 1(3) |
| Moderate | 0 | 0 |
| Lower GI bleed** | | |
| Severe | 0 | 1(2.5) |
| Non-GI: | | |
| Dizziness | | |
| Mild | 0 | 7(20.5) |
| Moderate | 0 | 1(2.9) |
| Blood in urine* | | |
| Mild | 1(10) | 0 |
| Moderate | 0 | 0 |
| Headache | | |
| Mild | 1(10) | 3(8.8) |
| Moderate | 0 | 0 |
| Urinary retention | | |
| Mild | 0 | 1(3) |
| Dosed | 10 | 34 |
| Moderate | 0 | 0 |
| Urinary tract infection | | |
| Mild | 0 | 1(3) |
| Moderate | 0 | 2(5.8) |
| Increased urinary frequency | | |
| Mild | 0 | 2(5.8) |
| Moderate | 0 | 0 |
| Skin lesions-rash | | |
| Mild | 0 | 3(8.8) |
| Moderate | 0 | 0 |
| Eye infection | | |
| Mild | 0 | 1(3) |
| Moderate | 0 | 0 |
| Difficulty falling asleep | | |
| Mild | 0 | 1(3) |
| Moderate | 0 | 0 |

*Unrelated to ENT-01
**colonic diverticulosis, polyp, patient on aspirin, Plavix and naproxen. Unrelated to ENT-01

TABLE 5
Common adverse events by dose

| Dose | Stage 1 | | | Stage 2 | | |
|---|---|---|---|---|---|---|
| (mg) | Diarrhea | Nausea | Vomiting | Diarrhea | Nausea | Dizziness* |
| 0 | 0 | 0 | 0 | 1 | 0 | 2 |
| 25 | 1 | 0 | 0 | — | — | — |
| 50 | 1 | 0 | 0 | — | — | — |
| 75 | 1 | 0 | 0 | 7 | 3 | 8 |
| 100 | 0 | 1 | 1 | 10 | 12 | 7 |
| 125 | 1 | 2 | 1 | 3 | 4 | 8 |
| 150 | 1 | 0 | 0 | 2 | 11 | 2 |
| 175 | 1 | 1 | 0 | 1 | 12 | 0 |
| 200 | 0 | 2 | 0 | 3 | 6 | — |
| 225 | — | — | — | 3 | 1 | |
| 250 | — | — | — | 2 | — | |

*lightheadedness included

TABLE 6
Dose limiting toxicity criteria

| | |
|---|---|
| Diarrhea | Increase 4-6 stools/day over baseline |
| Vomiting | 3-5 episodes in 24 hours |
| Abdominal pain | Moderate pain limiting daily activities |
| Postural hypotension | Moderately symptomatic and limiting daily |
| | activities or BP <80/40 |

No formal sample size calculation was performed for Stage 1. The number of subjects (n=10) was based on feasibility and was considered sufficient to meet the objectives of the study; which was to determine the tolerability of the treatment across the range of tested doses. For Stage 2, assuming the highest proportion of spontaneous resolution of constipation with no treatment to be 0.10, 34 evaluable subjects who have measurements at both baseline and at the end of the fixed dose period provided 80% power to detect the difference between 0.10 (proportion expected if patients are not treated) and a squalamine (ENT-01) treated proportion of 0.29.

No randomization was performed for Stage 1. During the randomization period of Stage 2, subjects were randomly allocated in equal proportion (1:1) to 1 of 2 double-blind treatment groups in a block size of 4: (1) squalamine (ENT-01) at the identified fixed dose level, or (2) placebo at the identified fixed dose level.

Adverse events were coded using the current version of MedDRA. Severity of AEs were assessed by investigators according to CTCAE (v4.03): Grade 1 is labeled as Mild, Grade 2 as Moderate, and Grade 3 and above as Severe. AEs that have a possible, probable or definite relationship to study drug were defined to be related to the study drug while others were defined as “not related.” The number (percentage) of subjects who experienced an AE during escalation and fixed dosing periods were summarized by dose level and overall for each stage. The denominator for calculating the percentages were based on the number of subjects ever exposed to each dose and overall.

Effect on Bowel Function:

Cumulative responder rates of bowel function are shown in FIG. 1A. In Stage 1 (single dose), cumulative response rate increased in a dose-dependent fashion from 25% at 25 mg to a maximum of 80% at 200 mg.

In Stage 2 (daily dosing), the response rate increased in a dose-dependent fashion from 26% at 75 mg to 85.3% at 250 mg. The dose required for a bowel response was patient-specific and varied from 75 mg to 250 mg. Median efficacious dose was 100 mg. Average CSBM/week increased from 1.2 at baseline to 3.8 at fixed dose (p=2.3×10⁻⁸) and SBM increased from 2.6 at baseline to 4.5 at fixed dose (p=6.4×10⁻⁶) (Table 7). Use of rescue medication decreased from 1.8/week at baseline to 0.3 at fixed dose (p=1.33×10⁻⁵). Consistency based on the Bristol stool scale also improved, increasing from mean 2.7 to 4.1 (p=0.0001) and ease of passage increased from 3.2 to 3.7 (p=0.03). Subjective indices of wellbeing (PAC-QOL) and constipation symptoms (PAC-SYM) also improved during treatment (p=0.009 and p=0.03 respectively).

TABLE 7
Stool related indices Stage 2 (Dosed patients, n = 34)

| | Baseline | Fixed dose | |
|---|---|---|---|
| | (mean, SD) | (mean, SD) | P-value |
| CSBM* | 1.2 (0.90) | 3.8 (2.40) | 2.3 × 10⁻⁸ |
| SBM* | 2.6 (1.45) | 4.5 (2.21) | 6.4 × 10⁻⁶ |
| Suppository use* | 1.8 (1.92) | 0.3 (0.67) | 1.33 × 10⁻⁵ |
| Consistency*** | 2.7 (1.20) | 4.1 (2.13) | 0.0001 |
| Ease of passage** | 3.2 (0.73) | 3.7 (1.19) | 0.03 |
| PAC-QOL total | 1.4 (0.49) | 1.2 (0.59) | 0.009 |
| PAC-SYM | 1.3 (0.45) | 1.1 (0.49) | 0.03 |

*weekly average;
**Ease of evacuation scale, where 1-manual disimpaction and 7 = incontinent;
***Bristol stool scale 1-7, where 1 = separate hard lumps and 7 = liquid consistency

The dose that proved efficacious in inducing a bowel response was strongly related to constipation severity at baseline (p=0.00055) (FIG. 1B); patients with baseline constipation of <1 CSBM/week required higher doses for a response (mean 192 mg) than patients with ≥1 CSBM/week (mean 120 mg).

While the improvement in most stool-related indices did not persist beyond the treatment period, CSBM frequency remained significantly above baseline value (Table 8).

TABLE 8
Reversal of stool indices to baseline during the wash-out period (Stage 2)

| | | | | P-value |
|---|---|---|---|---|
| | Baseline | Fixed dose | Wash-out | (wash-out vs. |
| | (Mean, SD) | (Mean, SD) | (Mean, SD) | baseline) |
| CSBM | 1.2 (0.90) | 3.8 (2.4) | 1.8 (1.19) | 0.01 |
| SBM | 2.6 (1.45) | 4.5 (2.21) | 3.2 (1.80) | 0.16 |
| Ease | 3.2 (0.73) | 3.7 (1.19) | 3.3 (0.81) | 0.78 |
| Consistency | 2.7 (1.20) | 4.1 (2.13) | 2.8 (1.39) | 0.85 |
| Rescue meds | 1.8 (1.92) | 0.3 (0.67) | 1.0 (1.40) | 0.13 |
| PAQ-QOL | 1.4 (0.49) | 1.2 (0.59 | 1.2 (0.63) | 0.04 |
| PAQ-SYM | 1.3 (0.45) | 1.1 (0.49) | 1.1 (0.60) | 0.11 |

The primary efficacy outcome variable was whether or not a subject was a “success” or “failure.” This is an endpoint based on subject diary entries for the “fixed dose” period prior to the endpoint assessment defined as average complete stool frequency increase by 1 or more over baseline, or 3 or more complete spontaneous stools/week. The subject was deemed a “success” if s/he met one or more of the criteria listed above, otherwise the subject was deemed a “failure.” The primary analysis was based on all subjects with a baseline assessment and an assessment at the end of the “fixed-dose” period and was a comparison of the proportion of successes with 0.10 (the null hypothesis corresponding to no treatment effect).

The proportion of subjects for whom the drug was a success was estimated with a binomial point estimate and corresponding 95% confidence interval. A secondary analysis compared the proportions of subjects who are deemed a success at the end of the randomized fixed-dose period between those randomized to the squalamine (ENT-01) arm and those randomized to the placebo arm. A Fisher's exact test was used to compare the proportions of subjects who were deemed a success at the end of randomization period between the two randomized arms

Subgroup Analysis:

Fifteen patients were randomized to treatment (n=6) or placebo (n=9) after the fixed dose period. During the 4-6 days of randomized treatment, the mean CSBM frequency in the treatment group remained higher than baseline as compared to those receiving placebo who returned to their baseline values (Table 9).

TABLE 9
CSBM frequency in the randomized cohort

| CSBM/week | Baseline | Fixed dose | Randomized | Washout |
|---|---|---|---|---|
| Treatment (n = 6) | 0.8 | 3.2 | 2.4 | 0.9 |
| Placebo (n = 9) | 1.6 | 3.3 | 1.4 | 1.6 |

CSBM increased in both groups during the treatment period and remained high in the treatment group during the randomized period but fell to baseline values in the placebo group.

Pharmacokinetics:

PK data were collected on the 10 patients enrolled in Stage 1 and 10 patients enrolled in Stage 2 to determine the extent of systemic absorption. In Stage 1, PK data were obtained at each visit, pre-medication, at 1, 2, 4, 8 and 24 hours (Table 10). In Stage 2, PK was measured on days 1 and 6 of the randomization period pre-medication, at 1, 2, 4 and 8 hours (Table 11). Based on the pharmacokinetic behavior of intravenously administered squalamine determined in prior clinical studies it is estimated that squalamine (ENT-01) exhibited oral bioavailability of less than 0.3% (Bhargava et al., 2001; Hao et al., 2003).

TABLE 10
Pharmacokinetics of orally administered
squalamine (ENT-01) in Stage 1.
Stage 1

| | | | Tₘₐₓ (hour) | T_{1/2} | AUC_{0-8 hr} | AUC_{0-16 hr} |
|---|---|---|---|---|---|---|
| Dose | # of | Cₘₐₓ | (Median | (hours) | (ng * | (ng * |
| (mg) | patients | (ng/ml) | Value) | (n) | hour/ml | hour/ml |
| 25 | 9 | 2.84 | 1.0 | 2.6 (3) | 10.8 | 19.6 |
| 50 | 10 | 3.73 | 2.0 | 3.4 (3) | 18.5 | 33.1 |
| 75 | 9 | 4.33 | 2.0 | 2.8 (2) | 18.4 | 29.8 |
| 100 | 9 | 6.18 | 2.0 | 3.9 (5) | 29.6 | 51.5 |
| 125 | 9 | 9.63 | 2.0 | 3.9 (4) | 43.1 | 77.7 |
| 150 | 7 | 6.27 | 2.0 | 5.6 (4) | 31.5 | 64.0 |
| 175 | 7 | 10.3 | 2.0 | 9.1 (6) | 49.7 | 91.2 |
| 200 | 6 | 15.1 | 2.0 | 9.0 (5) | 78.3 | 157 |

TABLE 11
Pharmacokinetics of orally administered squalamine
(ENT-01) in Stage 2.
Stage 2

| | # of | | | | |
|---|---|---|---|---|---|
| | patients | | Tₘₐₓ (hour) | T_{1/2} | |
| Dose | (2 visits | Cₘₐₓ | (Median | (hours) | AUC_{0-8 hr} |
| (mg) | each) | (ng/ml) | Value) | (n) | (ng * hour/ml |
| 75 | 1 | 10.0 | 3.0 | 5.5 (1) | 59.0 |
| 100 | 4 | 17.7 | 1.0 | 4.8 (5) | 70.3 |
| 125 | | | | | |
| 150 | | | | | |
| 175 | 5 | 11.8 | 2.0 | 10 (6) | 66.8 |

The mean Cₘₐₓ, Tₘₐₓ and T_{1/2} and AUC of the squalamine ion following squalamine (ENT-01) oral dosing for Stage 1 patients. The PK analyses are only approximate, as the lower limit of the validated concentration range was 10 ng/ml; most of the measured concentrations fell below that value. The mean Cₘₐₓ, Tₘₐₓ and T_{1/2} and AUC of the squalamine ion following squalamine (ENT-01) oral dosing for Stage 2 patients. The PK analyses are only approximate, as the lower limit of the validated concentration range was 0.5 ng/ml.

CNS Symptoms in Stage 2:

An exploratory analysis was done with respect to the sleep data, the body temperature data, mood, fatigue, hallucinations, cognition and other motor and non-motor symptoms of PD. Continuous measurements within a subject were compared with a paired t-test and continuous measurements between subject groups were compared with a two-group t-test. Categorical data were compared with a chi-squared test or a Fisher's exact test if the expected cell counts are too small for a chi-squared test.

CNS Symptoms:

CNS symptoms were evaluated at baseline and at the end of the fixed dose period and the wash-out period (Table 12). Total UPDRS score was 64.4 at baseline, 60.6 at the end of the fixed dose period and 55.7 at the end of the wash-out period (p=0.002); similarly, the motor component of the UPDRS improved from 35.3 at baseline to 33.3 at the end of fixed dose to 30.2 at the end of wash-out (p=0.006). MMSE improved from 28.4 at baseline to 28.7 during treatment and to 29.3 during wash-out (p=0.0006). BDI-II decreased from 10.9 at baseline to 9.9 during treatment and 8.7 at wash-out (p=0.10). PDHQ improved from 1.3 at baseline to 1.8 during treatment and 0.9 during wash-out (p=0.03). Hallucinations were reported by κ patients at baseline and delusions in 1 patient. Both hallucinations and delusions improved or disappeared in 5 of 6 patients during treatment and did not return for 4 weeks following discontinuation of squalamine (ENT-01) in 1 patient and 2 weeks in another. The frequency of arm or leg thrashing reported in the sleep diary diminished progressively from 2.2 episodes/week at baseline to 0 at maximal dose. Total sleep time increased progressively from 7.1 hours at baseline to 8.4 hours at 250 mg and was consistently higher than baseline beyond 125 mg (FIG. 4). Unlike stool-related indices, the improvement in many CNS symptoms persisted during wash-out.

TABLE 12
Effect of Squalamine (ENT-01) on neurological symptoms (n = 34)

| | Baseline | Fixed dose | | Wash-out | |
|---|---|---|---|---|---|
| UPDRS | (Mean, SD) | (Mean, SD) | P-value | (Mean, SD) | P-value |
| Part 1 (NMS) | 11.6 (6.51) | 10.6 (6.18)) | 0.28 | 9.5 (5.27) | 0.06 |
| Part 2 (Daily living) | 14.9 (8.11) | 14.7 (9.02) | 0.77 | 14.1 (8.21) | 0.40 |
| Part 3 (Motor) | 35.3 (14.35) | 33.3 (15.20) | 0.13 | 30.2 (13.23) | 0.005 |
| Total | 64.4 (23.72) | 60.6 (25.60) | 0.09 | 55.7 (23.69) | 0.002 |
| MMSE | 28.4 (1.75) | 28.7 (1.9) | 0.21 | 29.3 (1.06) | 0.0006 |
| PDHQ | 1.3 (2.99) | 1.8 (3.34) | 0.45 | 0.9 (2.33) | 0.03 |
| BDI-II | 10.9 (7.12) | 9.9 (6.45) | 0.14 | 8.7 (5.19) | 0.10 |

UPDRS: Unified Parkinson's Disease Severity Score; NMS: Non-motor symptoms; BDI: Beck Depression Index-II; MMSE: Mini-mental State exam. PDHQ: Parkinson's Disease Hallucination Questionnaire

Circadian rhythm of skin temperature was evaluable in 12 patients (i.e., those who had recordings that extended from baseline through washout). Circadian system functionality was evaluated by continuously monitoring wrist skin temperature using a temperature sensor (Thermochron iButton DS1921H; Maxim, Dallas, Tex.) (Sarabia et al. 2008). A nonparametric analysis was performed for each participant to characterize DST as previously described (Sarabia et al. 2008; Ortiz-Tudela et al. 2010).

Briefly, this analysis includes the following parameters: (i) the inter-daily stability (the constancy of 24-hour rhythmic pattern over days, IS); (ii) intra-daily variability (rhythm fragmentation, IV); (iii) average of 10-minute intervals for the 10 hours with the minimum temperature (L10); (iv) average of 10-minute intervals for the 5 hours with the maximum temperature (M5) and the relative amplitude (RA), which was determined by the difference between M5 and L10, divided by the sum of both. Finally, the Circadian Function Index (CFI) was calculated by integrating IS, IV, and RA. Consequently, CFI is a global measure that oscillates between 0 for the absence of circadian rhythmicity and 1 for a robust circadian rhythm (Ortiz-Tudela et al., 2010).

A comparison was performed of circadian rhythm parameters during the baseline, fixed dose and washout periods. ENT-01 administration improved all markers of healthy circadian function, increasing rhythm stability (IS, p=0.026), relative amplitude (RA, p=0.001) and circadian function index (CFI, p=0.016), while reducing rhythm fragmentation (IV, p=0.031). The improvement persisted for several of these circadian parameters during wash-out period (IS, p=0.008 and CFI, p=0.004). (FIG. 5).

Conclusions:

This Phase 2 trial involving 50 patients with PD assessed the safety of orally administered ENT-01, and the effect on bowel function and neurologic symptoms of PD. In addition, the study aimed to identify a dose of ENT-01 that normalizes bowel function in each patient. The study achieved the objectives of identifying safety and pharmacodynamics responses of ENT-01 in PD. In addition, the study is the first proof of concept demonstration that directly targeting αS pharmacologically can achieve beneficial GI, autonomic and CNS responses.

The effective dose ranged between 75 mg and 250 mg, with 85% of patients responding within this range. This dose correlated positively with constipation severity at baseline consistent with the hypothesis that gastrointestinal dysmotility in PD results from the progressive accumulation of αS in the ENS, and that squalamine (ENT-01) can restore neuronal function by displacing αS and stimulating enteric neurons. These results demonstrate that the ENS in PD is not irreversibly damaged and can be restored to normal function.

Several exploratory endpoints were incorporated into the trial to evaluate the impact of ENT-01 on neurologic symptoms associated with PD. The UPDRS score, a global assessment of motor and non-motor symptoms, showed significant improvement. Improvement was also seen in the motor component. The improvement in the motor component is unlikely to be due to improved gastric motility and increased absorption of dopaminergic medications, since improvement persisted during the 2-week wash-out period, i.e., in the absence of study drug (Table 12).

Improvements were also seen in cognitive function (MMSE scores), hallucinations, REM-behavior disorder (RBD) and sleep. Six of the patients enrolled had daily hallucinations or delusions and these improved or disappeared during treatment in five. In one patient the hallucinations disappeared at 100 mg, despite not having reached the colonic prokinetic dose at 175 mg. The patient remained free of hallucinations for 1 month following cessation of dosing. RBD and total sleep time also improved progressively in a dose-dependent manner.

The prokinetic effect of the amino sterol squalamine appears to occur through local action of the compound on the ENS, since squalamine, the active zwitterion, is not significantly absorbed into the systemic circulation.

### Example 2—Constipation

This prophetic example describes an exemplary method of (i) treating constipation associated with ASD and/or (ii) treating and/or preventing ASD in which constipation is a known symptom in a subject.

ASD patients are selected based on the constipation criteria described in Example 1. The patients are then subdivided into a control subgroup and a treatment subgroup. A “fixed dose” of an aminosterol or a salt or derivative thereof for each of the patients in the treatment subgroup is determined using the method described in Example 1 and in the application supra. Treatment and wash-out periods mirror Example 1. ASD patients are monitored for changes in the severity or occurrence of the symptoms. ASD patients are also monitored for changes in other symptoms associated with ASD.

ASD patients having more severe constipation, e.g., less than 1 spontaneous bowel movement per week, are started at a dose of 75 mg/day or more. ASD patients having less severe constipation, e.g., 1 or more SBM/week, are started at a lower dose of amino sterol, e.g., a starting dose of less than 75 mg/day, for example a dose of 25 mg/day. Thus, the starting aminosterol dose is dependent upon constipation severity. The full aminosterol dosing range is from about 1 to about 500 mg/day. Once a fixed aminosterol dose has been identified for an ASD patient, the ASD subject is started at that same dose following drug cessation and reintroduction of drug dosing; e.g., there is no need to ramp up dosing once a fixed aminosterol dose for a patient has been identified.

### Example 3—Avoidance of Eye Contact

This prophetic example describes an exemplary method of (i) treating avoidance of eye contact associated with ASD and/or (ii) treating and/or preventing ASD in which avoidance of eye contact is a known symptom.

ASD patients are selected based on displaying avoidance of eye contact. The patients are then subdivided into a control subgroup and a treatment subgroup. A “fixed dose” of an aminosterol or a salt or derivative thereof for each of the patients in the treatment subgroup is determined using the method described in Example 1, using the improvement (increase) in eye contact as an endpoint. Increase in eye contact may include an increases in the number of instances in which the patient engages in eye contact and/or an increase in the amount of time in which the patient stays engaged in eye contact. Treatment and wash-out periods mirror Example 1. ASD patients are monitored for changes in the severity or occurrence of the symptoms.

### Example 4—Sleep Disorder or Sleep Disturbance—Decreased Quantity of REM Sleep

This prophetic example describes an exemplary method of (i) treating decreased quantity of REM sleep in ASD patients and/or (ii) treating and/or preventing ASD in which decreased quantity of REM sleep is a known symptom in an ASD subject having decreased quantity of REM sleep.

ASD patients are selected based on having decreased quantity of REM sleep. The patients are then subdivided into a control subgroup and a treatment subgroup. A “fixed dose” of an aminosterol or a salt or derivative thereof for each of the ASD patients in the treatment subgroup is determined using the method described in Example 1, using improvement (increase) in REM sleep as an endpoint. Treatment and wash-out periods mirror Example 1. ASD patients are monitored for changes in the severity or occurrence of the symptom.

### Example 5—Echolalia

This prophetic example describes an exemplary method of (i) treating echolalia and/or palilalia in an ASD subject and/or (ii) treating and/or preventing ASD in which engagement in echolalia and/or palilalia is a known symptom in an ASD subject that engages in echolalia and/or palilalia.

ASD patients are selected based on engagement in echolalia and/or palilalia. The patients are then subdivided into a control subgroup and a treatment subgroup. A “fixed dose” of an aminosterol or a salt or derivative thereof for each of the ASD patients in the treatment subgroup is determined using the method described in Example 1, using the improvement (decrease in instances) of echolalia and/or palilalia symptoms as an endpoint. Treatment and wash-out periods mirror Example 1. Patients are monitored for changes in the severity or occurrence of the symptoms.

### Example 6—Self-Injury

This prophetic example describes an exemplary method of (i) treating self-injury in an ASD subject and/or (ii) treating and/or preventing ASD in which self-injury is a known symptom in an ASD subject that engages in self-injury.

ASD patients are selected based on engagement in self-injury. The patients are then subdivided into a control subgroup and a treatment subgroup. A “fixed dose” of an amino sterol or a salt or derivative thereof for each of the ASD patients in the treatment subgroup is determined using the method described in Example 1, using the improvement (decrease in instances) of self-injury as an endpoint. Treatment and wash-out periods mirror Example 1. Patients are monitored for changes in the severity or occurrence of the symptoms.

### Example 7—Repetitive Motion

This prophetic example describes an exemplary method of (i) treating repetitive motion in an ASD subject and/or (ii) treating and/or preventing ASD in which repetitive motion is a known symptom in an ASD subject that engages in repetitive motion.

ASD patients are selected based on engagement in repetitive motion. The patients are then subdivided into a control subgroup and a treatment subgroup. A “fixed dose” of an aminosterol or a salt or derivative thereof for each of the ASD patients in the treatment subgroup is determined using the method described in Example 1, using the improvement (decrease in instances) of repetitive motion as an endpoint. Treatment and wash-out periods mirror Example 1. Patients are monitored for changes in the severity or occurrence of the symptoms.

### Example 8—ASD

This prophetic example describes an exemplary method of treating and/or preventing ASD in a subject in need thereof.

ASD patients are selected based on being diagnosed with ASD, i.e., having ASD or exhibiting known risk factors of ASD, i.e., familial history and/or genetic risk of ASD. Patients are grouped based on having ASD or at risk for developing ASD. The groups are then subdivided into a control subgroup and a treatment subgroup. A “fixed dose” of an amino sterol or a salt or derivative thereof for each of the patients in the treatment subgroup is determined using the method described in Example 1, using either the improvement of constipation or another symptom of ASD as an endpoint. Treatment and wash-out periods mirror Example 1. Patients are monitored for changes in the severity or occurrence of the symptoms. Patients having ASD are monitored for changes in other symptoms associated with the disorder. Patients at risk for developing ASD are monitored for the development of AD.

### Example 9—Cognitive Impairment

This prophetic example describes an exemplary method of (i) treating cognitive impairment in ASD subjects/or (ii) treating and/or preventing ASD in which cognitive impairment is a known symptom.

ASD patients are selected based on having cognitive impairment. The patients are then subdivided into a control subgroup and a treatment subgroup. A “fixed dose” of an amino sterol or a salt or derivative thereof for each of the patients in the ASD treatment subgroup is determined using the method described in Example 1, using the improvement of cognitive impairment symptoms as an endpoint. Treatment and wash-out periods mirror Example 1. Patients are monitored for changes in the severity or occurrence of the symptoms. ASD patients are also monitored for changes in other symptoms associated with AD.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, or compositions, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

### REFERENCES

Albert et al., “The Diagnosis of Mild Cognitive Impairment Due to Alzheimer's Disease: Recommendations from the National Institute on Aging—Alzheimer's Association Workgroups on Diagnostic Guidelines for Alzheimer's Disease,” Alzheimer's & Dementia, 7(3):270-279 (2011).
Andresen et al., “Effect of 5 days linaclotide on transit and bowel function in females with constipation-predominant irritable bowel syndrome,” Gastroenterology, 133:761-8 (2007).
Antonio-Rubio et al., “Abnormal thermography in Parkinson's disease,” Parkinsonism Relat. Disord., 21:852-7 (2015).
“Autism Spectrum Disorder (ASD)—NCBDDD—CDC”. https://www.cdc.gov/ncbddd/autism/index.html, 4 May 2018
Bhargava et al., “A phase I and pharmacokinetic study of squalamine, a novel antiangiogenic agent, in patients with advanced cancers,” Clin. Cancer Res., 7:3912-9 (2001).
Breen et al., “Sleep and circadian rhythm regulation in early Parkinson disease,” JAMA Neurol., 71:589-95 (2014).
Cheng et al., “The role of alpha synuclein in neurotransmission and synaptic plasticity,” J. of Chem. Neuroanatomy, 42(4):242-248 (2011).
Cordell et al., “Alzheimer's Association Recommendations for Operationalizing the Detection of Cognitive Impairment During the Medicare Annual Wellness Visit in a Primary Care Setting,” Alzheimer's & Dementia, 9(2):141-150 (2013).
Emerson et al., “Functional neuroimaging of high-risk 6-month-old infants predicts a diagnosis of autism at 24 months of age” Science Translational Medicine 7 Jun. 2017: Vol. 9, Issue 393.
Fahn S E R, Members of the UPDRS Development Committee. UNIFIED PARKINSON'S DISEASE RATING SCALE. Florham Park, N.J.: Macmillan Health Care Information (1987).
Frank et al., “Psychometric validation of a constipation symptom assessment questionnaire,” Scand. J. Gastroenterol., 34:870-7 (1999).
Frith et al., “Autism spectrum disorder” Current Biology. 15 (19): R786-R790 2005.
Glessner et al., “Autism-wide copy number variation reveals ubiquitin and neuronal genes,” Nature, 459(7246):569-573 (2009).
Hadjikhani et al., “Look me in the eyes: constraining gaze in the eye-region provokes abnormally high subcortical activation in autism” Scientific Reports volume 7, Article number: 3163 (2017).
Hao et al., “A Phase I and pharmacokinetic study of squalamine, an amino sterol angiogenesis inhibitor,” Clin. Cancer Res., 9:2465-71 (2003).
Heaton et al., “Defecation frequency and timing, and stool form in the general population: a prospective study,” Gut, 33:818-24 (1992).
Jack et al., “Introduction to the Recommendations from the National Institute on Aging—Alzheimer's Association Workgroups on Diagnostic Guidelines for Alzheimer's Disease,” Alzheimer's & Dementia, 7(3):257-262 (2011).
Kadak et al., “Low Serum Level alpha-synuclein and Tau Protein in Autism Spectrum Disorder Compared to Controls,” Neuropediatrics., 46(6):410-415 (2015).
Kirkevold, O. & Selbaek, G., “The Agreement Between the MMSE and IQCODE Tests in a Community-Based Sample of Subjects Aged 70 Years or Older Receiving In-Home Nursing: An Explorative Study,” Dement Geriatr. Cogn. Dis. Extra, 5(1):32-41 (2015).
Leigh et al., “Brief Report: Forecasting the Economic Burden of Autism in 2015 and 2025 in the United States” J. Autism Dev Disord. 45(12):4135-9 (2015).
Lewis S J, Heaton K W., “Stool form scale as a useful guide to intestinal transit time,” Scand. J. Gastroenterol., 32:920-4 (1997).
Madrid-Navarro et al., “Multidimensional Circadian Monitoring by Wearable Biosensors in Parkinson's Disease,” Front. Neurol., 9:157 (2018).
Mahlknecht et al., “Prodromal Parkinson's Disease as Defined per MDS Research Criteria in the General Elderly Community,” Mov. Disord., 31:1405-1408 (2016). Marquis et al., “Development and validation of the Patient Assessment of Constipation Quality of Life questionnaire,” Scand. J. Gastroenterol., 40:540-51 (2005).
Mash & Barkley (2003). Child Psychopathology. New York: The Guilford Press. pp. 409-454.
Mearin et al., “Bowel Disorders,” Gastroenterology, 150(6):1393-1407 (2016).
McKhann et al., “The Diagnosis of Dementia Due to Alzheimer's Disease: Recommendations from the National Institute on Aging—Alzheimer's Association Workgroups on Diagnostic Guidelines for Alzheimer's Disease,” Alzheimer's & Dementia, 7(3):263-269 (2011).
Newschaffer C J, Croen L A, Daniels J, et al. The epidemiology of autism spectrum disorders. Annu Rev Public Health. 2007; 28:235-58.
Ortiz-Tudela et al., “Ambulatory circadian monitoring (ACM) based on thermometry, motor activity and body position (TAP): a comparison with polysomnography,” Physiol. Behay., 126:30-8 (2014).
Palsetia et al., “The Clock Drawing Test versus Mini-mental Status Examination as a Screening Tool for Dementia: A Clinical Comparison,” Indian J. Psychol. Med., 40:1-10 (2018).
Papapetropoulos et al., “A questionnaire-based (UM-PDHQ) study of hallucinations in Parkinson's disease,” BMC Neurol., 8:21 (2008).
Perni et al., “A natural product inhibits the initiation of alpha-synuclein aggregation and suppresses its toxicity,” PNAS, USA, 114:E1009-E17 (2017).
Sarabia et al., “Circadian rhythm of wrist temperature in normal-living subjects A candidate of new index of the circadian system,” Physiol. Behay., 95:570-80 (2008).
Shehata et al., “Neuronal stimulation induces autophagy in hippocampal neurons that is involved in AMPA receptor degradation after chemical long-term depression,” J. Neurosci., 32:10413-22 (2012).
Soke et al. J. Autism Dev. Disord. 46, 3607-3614 (2016)
W. Sriwimol and P. Limprasert, “Significant Changes in Plasma Alpha-Synucelin and Beta-Synuclein Levels in Male Children with Autism Spectrum Disorder,” BioMed Res. Int., vol. 2018, Article ID 4503871 (2018).
Starkstein et al., “High rates of parkinsonism in adults with autism” Journal of Neurodevelopmental Disorders Advancing Interdisciplinary Research, 7:29 (2015)
Steer et al., “Use of the Beck Depression Inventory-II with depressed geriatric inpatients,” Behay. Res. Ther., 38:311-8 (2000).
Stiasny-Kolster et al., “The REM sleep behavior disorder screening questionnaire—a new diagnostic instrument,” Movement disorders: Official J. of the Movement Dis. Soc., 22:2386-93 (2007).
Videnovic A, Golombek D., “Circadian Dysregulation in Parkinson's Disease,” Neurobiol. Sleep Circadian Rhythms, 2:53-8 (2017).
Zinsmeister et al., “Pharmacodynamic and clinical endpoints for functional colonic disorders: statistical considerations,” Dig. Dis. Sci., 58:509-18 (2013).
Volkmar, et al., (2005). Handbook of Autism and Pervasive Developmental Disorders (Third ed.). John wiley and Sons. p. 255-2557.
Yun et al., “Identification of squalamine in the plasma membrane of white blood cells in the sea lamprey, Petromyzon marinus” December 2007 The Journal of Lipid Research, 48, 2579-2586.

What is claimed is:

1. A method of treating, preventing, and/or slowing the onset or progression of autism spectrum disorder (ASD) and/or a related symptom in a subject in need comprising administering to the subject a therapeutically effective amount of at least one amino sterol, or a salt or derivative thereof, provided that the administering does not comprise oral administration.

2. The method of claim 1, wherein administering comprises a method of administration selected from nasal, sublingual, buccal, rectal, vaginal, intravenous, intra-arterial, intradermal, intraperitoneal, intrathecal, intramuscular, epidural, intracerebral, intracerebroventricular, transdermal, or any combination thereof.

3. The method of claim 1, wherein the method of administration comprises nasal administration.

4. The method of claim 1, wherein the therapeutically effective amount of at least one amino sterol, or a salt or derivative thereof:
(a) comprises about 0.1 to about 20 mg/kg body weight of the subject; and/or
(b) comprises about 0.1 to about 15 mg/kg body weight of the subject; and/or
(c) comprises about 0.1 to about 10 mg/kg body weight of the subject; and/or
(d) comprises about 0.1 to about 5 mg/kg body weight of the subject; and/or
(e) comprises about 0.1 to about 2.5 mg/kg body weight of the subject; and/or
(f) comprises about 0.001 to about 500 mg/day; and/or
(g) comprises about 0.001 to about 250 mg/day; and/or
(h) comprises about 0.001 to about 125 mg/day; and/or
(i) comprises about 0.001 to about 50 mg/day; and/or
(j) comprises about 0.001 to about 25 mg/day; and/or
(k) comprises about 0.001 to about 10 mg/day; and/or
(l) comprises about 0.001 to about 6 mg/day administered intranasal; and/or
(m) comprises about 0.001 to about 4 mg/day administered intranasal; and/or
(n) comprises about 0.001 to about 2 mg/day administered intranasal; and/or
(o) comprises about 0.001 to about 1 mg/day administered intranasal; and/or
(p) comprises about 1 to about 300 mg/day administered orally; and/or
(q) comprises about 25 to about 300 mg/day administered orally.

5. The method of claim 1, wherein:
(a) the aminosterol or a salt or derivative thereof is taken on an empty stomach, optionally within two hours of the subject waking; and/or
(b) no food is taken or consumed after about 60 to about 90 minutes of taking the aminosterol or a salt or derivative thereof; and/or
(c) the aminosterol or a salt or derivative thereof is a pharmaceutically acceptable grade of at least one aminosterol or a pharmaceutically acceptable salt or derivative thereof; and/or
(d) the aminosterol is comprised in a composition further comprising one or more of the following: an aqueous carrier; a buffer; a sugar; and/or a polyol compound; and/or
(e) the subject is human; and/or
(f) the subject is a member of a patient population or an individual at risk for developing ASD.

6. The method of claim 1, wherein the amino sterol or the salt or derivative thereof is:
(a) isolated from the liver of Squalus acanthias; and/or
(b) squalamine or a pharmaceutically acceptable salt thereof; and/or
(c) a squalamine isomer; and/or
(d) the phosphate salt of squalamine; and/or
(e) aminosterol 1436 or a pharmaceutically acceptable salt thereof; and/or
(f) an isomer of amino sterol 1436; and/or
(g) the phosphate salt of aminosterol 1436; and/or
(h) comprises a sterol nucleus and a polyamine attached at any position on the sterol, such that the molecule exhibits a net charge of at least +1; and/or
(i) comprises a bile acid nucleus and a polyamine, attached at any position on the bile acid, such that the molecule exhibits a net charge of at least +1; and/or
(j) a derivative modified to include one or more of the following:
(i) substitutions of the sulfate by a sulfonate, phosphate, carboxylate, or other anionic moiety chosen to circumvent metabolic removal of the sulfate moiety and oxidation of the cholesterol side chain;
(ii) replacement of a hydroxyl group by a non-metabolizable polar substituent, such as a fluorine atom, to prevent its metabolic oxidation or conjugation; and
(iii) substitution of one or more ring hydrogen atoms to prevent oxidative or reductive metabolism of the steroid ring system; and/or
(k) a derivative of squalamine modified through medicinal chemistry to improve bio-distribution, ease of administration, metabolic stability, or any combination thereof; and/or
(l) a synthetic amino sterol; and/or
(m) is selected from the group consisting of:

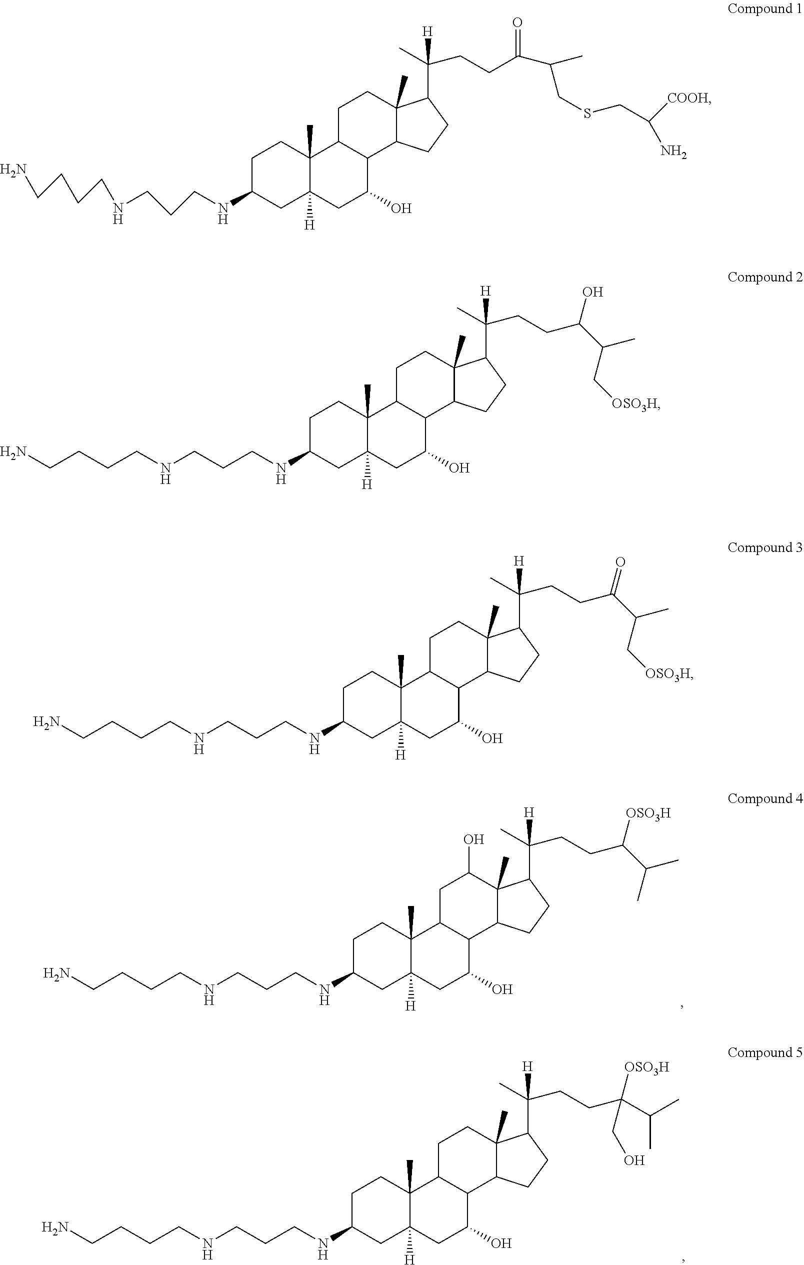

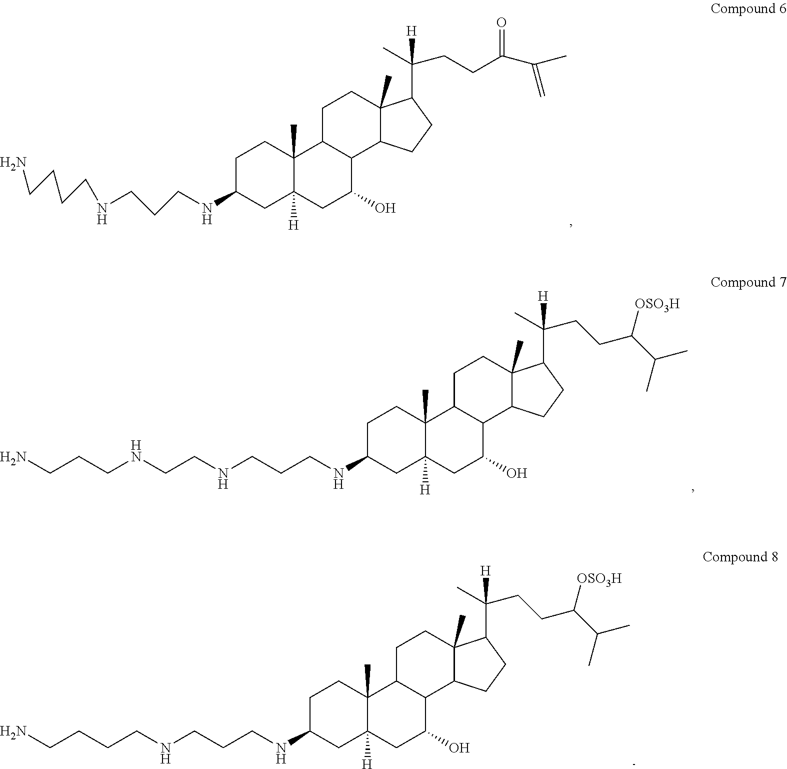

7. A method of treating, preventing, and/or slowing the onset or progression of autism spectrum disorder (ASD) and/or a related symptom in a subject in need comprising:
(a) determining a dose of an aminosterol or a salt or derivative thereof for the subject, wherein the aminosterol dose is determined based on the effectiveness of the aminosterol dose in improving or resolving an ASD symptom being evaluated,
(b) followed by administering the aminosterol dose to the subject for a defined period of time, wherein the method comprises:
(i) identifying an ASD symptom to be evaluated;
(ii) identifying a starting aminosterol dose for the subject; and
(iii) administering an escalating dose of the amino sterol to the subject over a defined period of time until an effective dose for the ASD symptom being evaluated is identified, wherein the effective dose is the aminosterol dose where improvement or resolution of the ASD symptom is observed, and fixing the aminosterol dose at that level for that particular ASD symptom in that particular subject; and
(c) optionally wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

8. The method of claim 7, wherein the amino sterol or a salt or derivative thereof is administered orally, intranasally, or a combination thereof.

9. The method of claim 8, wherein the amino sterol or a salt or derivative thereof is administered orally and:
(a) the starting dose of the aminosterol or a salt or derivative thereof ranges from about 1 mg up to about 175 mg/day; and/or
(b) the starting oral aminosterol dose is about 25 mg/day; and/or
(c) the dose of the amino sterol or a salt or derivative thereof for the subject following escalation is fixed at a range of from about 1 mg up to about 500 mg/day; and/or
(d) the dose of the amino sterol or a salt or derivative thereof for the subject following escalation is fixed at a dose of about 1, about 5, about 10, about 15, about 20, about 25, about 30, about 35, about 40, about 45, about 50, about 55, about 60, about 65, about 70, about 75, about 80, about 85, about 90, about 95, about 100, about 105, about 110, about 115, about 120, about 125, about 130, about 135, about 140, about 145, about 150, about 155, about 160, about 165, about 170, about 175, about 180, about 185, about 190, about 195, about 200, about 205, about 210, about 215, about 220, about 225, about 230, about 235, about 240, about 245, about 250, about 255, about 260, about 265, about 270, about 275, about 280, about 285, about 290, about 295, about 300, about 305, about 310, about 315, about 320, about 325, about 330, about 335, about 340, about 345, about 350, about 355, about 360, about 365, about 370, about 375, about 380, about 385, about 390, about 395, about 400, about 405, about 410, about 415, about 420, about 425, about 430, about 435, about 440, about 445, about 450, about 455, about 460, about 465, about 470, about 475, about 480, about 485, about 490, about 495, or about 500 mg/day; and/or
(e) the starting oral aminosterol dose is about 10, about 15, about 20, about 25, about 30, about 35, about 40, about 45, about 60, about 65, about 70, or about 75 mg/day; and/or
(f) the dose of the amino sterol or a salt or derivative thereof is escalated in about 25 mg increments; and/or
(g) the aminosterol or a salt or derivative thereof is formulated for oral administration in a composition which is a liquid, capsule, or tablet designed to disintegrate in either the stomach, upper small intestine, or more distal portions of the intestine.

10. The method of claim 8, wherein the amino sterol or a salt or derivative thereof is administered intranasally and:
(a) the starting dose of the aminosterol or a salt or derivative thereof ranges from about 0.001 mg to about 3 mg/day; and/or
(b) the dose of the amino sterol or a salt or derivative thereof for the subject following escalation is fixed at a range of from about 0.001 mg up to about 6 mg/day; and/or
(c) the dose of the aminosterol or a salt or derivative thereof for the subject following escalation is a dose which is subtherapeutic when administered orally or by injection; and/or
(d) the dose of the amino sterol or a salt or derivative thereof is escalated in increments of about 0.1, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, about 1, about 1.1, about 1.2, about 1.3, about 1.4, about 1.5, about 1.6, about 1.7, about 1.8, about 1.9, or about 2 mg; and/or
(e) the aminosterol or a salt or derivative thereof is formulated for intranasal administration in a composition which is a dry powder nasal spray or liquid nasal spray.

11. The method of claim 7, wherein the dose of the aminosterol or a salt or derivative thereof is escalated:
(a) every about 3 to about 5 days; and/or
(b) every about 1 to about 14 days; and/or
(c) every about 1, about 2, about 3, about 4, about 5, about 6, about 7, about 8, about 9, about 10, about 11, about 12, about 13, or about 14 days; and/or
(d) about 1×/week, about 2×/week, about every other week, or about 1×/month.

12. The method of claim 7, wherein the fixed dose of the aminosterol or a salt or derivative thereof:
(a) is administered once per day, every other day, once per week, twice per week, three times per week, four times per week, five times per week, six times per week, every other week, or every few days; and/or
(b) is administered for a first defined period of time of administration, followed by a cessation of administration for a second defined period of time, followed by resuming administration upon recurrence of ASD or a symptom of ASD; and/or
(c) is incrementally reduced after the fixed dose of aminosterol or a salt or derivative thereof has been administered to the subject for a defined period of time; and/or
(d) is varied plus or minus a defined amount to enable a modest reduction or increase in the fixed dose; and/or
(e) is varied plus or minus a defined amount to enable a modest reduction or increase in the fixed dose, and the fixed amino sterol dose is increased or decreased by about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%; and/or
(f) is administered once per day, every other day, once per week, twice per week, three times per week, four times per week, five times per week, six times per week, every other week, or every few days; and/or
(g) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

13. The method of claim 7, wherein:
(a) the starting dose of the aminosterol or a salt or derivative thereof is higher if the ASD-related symptom being evaluated is severe; and/or
(b) progression or onset of ASD is slowed, halted, or reversed over a defined period of time following administration of the fixed escalated dose of the aminosterol or a salt or derivative thereof, as measured by a medically-recognized technique; and/or
(c) the progression or onset of ASD is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or
(d) the ASD is positively impacted by the fixed escalated dose of the amino sterol or a salt or derivative thereof, as measured by a medically-recognized technique; and/or
(e) the positive impact on and/or progression of ASD is measured quantitatively or qualitatively by one or more techniques selected from the group consisting of computed tomography (CT), magnetic resonance imaging (MRI), magnetic resonance spectroscopy, functional MRI (fMRI), diffusion tensor imaging, single photon emission computed tomography (SPECT), and positron emission tomography (PET); and/or
(f) the fixed escalated dose of the amino sterol or a salt or derivative thereof reverses dysfunction caused by the ASD and treats, prevents, improves, and/or resolves the ASD-related symptom being evaluated; and/or
(g) the improvement or resolution of the ASD-related symptom is measured using a clinically recognized scale or tool; and/or
(h) the improvement or resolution of the ASD-related symptom is measured using a clinically recognized scale or tool and the clinical scale or tool is selected from the group consisting of the medically-recognized technique is one or more selected from the group consisting of Autism Spectrum Rating Scales (ASRS™), Autism Diagnostic Observation Schedule (ADOS), and Autism Diagnostic Interview-Revised (ADI-R); and/or
(i) the improvement in the ASD-related symptom is at least about 3%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 100%, as measured using a clinically recognized scale or tool; and/or
(j) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

14. The method of claim 7, wherein the symptom to be evaluated is selected from the group consisting of:
(a) a symptom from the Autism Spectrum Rating Scales (ASRS™) selected from the group consisting of social skills, communication skills, unusual behavior, self-regulation ability, peer socialization, adult socialization, atypical language, and stereotypy;
(b) a symptom from the Autism Diagnostic Observation Schedule (ADOS) selected from the group consisting of performance in Module 1, performance in Module 2, performance in Module 3, and performance in Module 4;
(c) a symptom from the Autism Diagnostic Interview-Revised (ADI-R) wherein the symptom is selected from the group consisting of emotional sharing, offering and seeking comfort, social smiling, responding to other children, stereotyped utterances, pronoun reversal, social usage of language, preoccupation with unusual things, hand and finger mannerism, unusual sensory interests, self-injury, aggression, and overactivity;
(d) failure to respond to name;
(e) failure to point at objects of interest;
(f) inability to role play;
(g) avoidance of eye contact;
(h) preference to be alone;
(i) inability to understand feelings of others;
(j) no speech or delayed development of speech;
(k) echolalia and/or palilalia;
(l) answering questions with unrelated answers;
(m) upset by minor changes;
(n) obsessive interests;
(o) lining-up or stacking of objects;
(p) repetitive motion;
(q) avoidance of physical contact with others;
(r) lack of awareness of danger;
(s) sleep disorder or sleep disturbance;
(t) constipation;
(u) cognitive impairment;
(v) gastrointestinal (GI) problems;
(w) epilepsy;
(x) feeding issues;
(y) Attention-deficit/hyperactivity disorder (ADHD);
(z) anxiety;
(aa) depression;
(bb) Obsessive compulsive disorder (OCD);
(cc) schizophrenia; and
(dd) Bipolar Disorder.

15. The method of claim 14, wherein the ASD symptom to be evaluated is a sleep disorder or sleep disturbance and wherein:
(a) the sleep disorder or sleep disturbance is selected from the group consisting of decreased quantity of REM sleep, increased undifferentiated sleep, immature organization of eye movements into discrete bursts during REM sleep, decreased time in bed, decreased total sleep time, decreased REM sleep latency, circadian rhythm disruption, and increased proportion of stage 1 sleep, or any combination thereof; and/or
(b) the method results in a positive change in the sleeping pattern of the subject over a defined period of time; and/or
(c) the method results in a positive change in the sleeping pattern of the subject, wherein the positive change is defined as:
(i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(d) as a result of the method the subject obtains the total number of hours of sleep recommended by a medical authority for the age group of the subject; and/or
(e) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

16. The method of claim 14, wherein the ASD symptom to be evaluated is avoidance of eye contact, and wherein:
(a) the method results in a positive change in the amount of eye contact engaged in by the subject over a defined period of time; and/or
(b) the method results in a positive change in the amount of eye contact engaged in by the subject over a defined period of time, wherein the positive change is defined as an increase in the amount of time of eye contact of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(c) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

17. The method of claim 14, wherein the ASD symptom to be evaluated is echolalia and/or palilalia and wherein:
(a) the method results in a decreased number of instances in which the subject engages in echolalia and/or palilalia over a defined period of time; and/or
(b) the method results in a decreased number of instances in which the subject engages in echolalia and/or palilalia over a defined period of time and the decrease is defined as a reduction in instances of engagement in echolalia and/or palilalia selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(c) the method results in the subject ceasing to engage in echolalia and/or palilalia; and/or
(d) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

18. The method of claim 14, wherein the ASD symptom to be evaluated is self-injury and wherein:
(a) the method results in a decreased number of instances in which the subject self-injures over a defined period of time; and/or
(b) the method results in a decreased number of instances in which the subject self-injures over a defined period of time, selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(c) the method results in the subject ceasing to self-injure; and/or
(d) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

19. The method of claim 14, wherein the ASD symptom to be evaluated is self-injury and wherein:
(a) the method results in a decreased severity of self-injuries over a defined period of time; and/or
(b) the method results in a decreased severity of self-injuries over a defined period of time, wherein the decrease in severity is selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(c) the decreased severity is measured using a medically recognized technique; and/or
(d) the method results in the subject ceasing to self-injure; and/or
(e) the medically recognized technique is one or more techniques selected from the group consisting of Barell Matrix, The Abbreviated Injury Scale, and the Glasgow Coma Scale; and/or
(f) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

20. The method of claim 14, wherein the ASD symptom to be evaluated is repetitive motion and wherein:
(a) the method results in a decreased number of instances in which the subject engages in repetitive motion over a defined period of time; and/or
(b) the method results in a decreased number of instances in which the subject engages in repetitive motion over a defined period of time, wherein the decrease in number is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(c) the method results in the subject ceasing to engage in repetitive motion; and/or
(d) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

21. The method of claim 14, wherein the ASD symptom to be evaluated is repetitive motion and wherein:
(a) the method results in a decreased severity of repetitive motion over a defined period of time; and/or
(b) the method results in a decreased severity of repetitive motion over a defined period of time, wherein the decrease in severity is by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(c) the method results in the subject ceasing to engage in repetitive motion; wherein the decrease in severity comprises a decrease in number of repetitions per minute (RPM) of the repetitive motion; and/or
(d) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

22. The method of claim 14, wherein the ASD symptom to be evaluated is constipation, and wherein:
(a) the fixed escalated aminosterol dose causes the subject to have a bowel movement; and/or
(b) the method results in an increase in the frequency of bowel movement in the subject over a defined period of time; and/or
(c) the method results in an increase in the frequency of bowel movement in the subject and the increase in the frequency of bowel movement is defined as:
(i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(d) as a result of the method the subject has the frequency of bowel movement recommended by a medical authority for the age group of the subject; and/or
(e) the starting aminosterol dose is determined by the severity of the constipation, wherein:
(i) if the average complete spontaneous bowel movement (CSBM) or spontaneous bowel movement (SBM) is one or less per week, then the starting amino sterol dose is at least about 150 mg; and
(ii) if the average CSBM or SBM is greater than one per week, then the starting aminosterol dose is about 75 mg or less; and/or
(f) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

23. The method of claim 14, wherein the ASD symptom to be evaluated is cognitive impairment, and wherein:
(a) progression or onset of the cognitive impairment is slowed, halted, or reversed over a defined period of time following administration of the fixed escalated dose of the aminosterol or a salt or derivative thereof, as measured by a medically-recognized technique; and/or
(b) the cognitive impairment is positively impacted by the fixed escalated dose of the aminosterol or a salt or derivative thereof, as measured by a medically-recognized technique; and/or
(c) the cognitive impairment is positively impacted by the fixed escalated dose of the aminosterol or a salt or derivative thereof, as measured by a medically-recognized technique and the positive impact on and/or progression of cognitive impairment is measured quantitatively or qualitatively by one or more techniques selected from the group consisting of ADASCog, Mini-Mental State Exam (MMSE), Mini-cog test, Woodcock-Johnson Tests of Cognitive Abilities, Leiter International Performance Scale, Miller Analogies Test, Raven's Progressive Matrices, Wonderlic Personnel Test, IQ tests, and a computerized tested selected from Cantab Mobile, Cognigram, Cognivue, Cognision, or Automated Neuropsychological Assessment Metrics Cognitive Performance Test (CPT); and/or
(d) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or
(e) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

24. The method of claim 14, wherein the ASD symptom to be evaluated is depression and wherein:
(a) treating the depression prevents and/or delays the onset and/or progression of ASD; and/or
(b) the method results in improvement in a subject's depression over a defined period of time, as measured by one or more clinically-recognized depression rating scale; and/or
(c) the method results in improvement in a subject's depression, as measured by one or more clinically-recognized depression rating scale and the improvement is in one or more depression characteristics selected from the group consisting of mood, behavior, bodily functions such as eating, sleeping, energy, and sexual activity, and/or episodes of sadness or apathy; and/or
(d) the method results in improvement in a subject's depression, as measured by one or more clinically-recognized depression rating scale, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%; and/or
(e) the one or more clinically-recognized depression rating scale is selected from the group consisting of Patient Health Questionnaire-9 (PHQ-9); the Beck Depression Inventory (BDI); Zung Self-Rating Depression Scale; Center for Epidemiologic Studies-Depression Scale (CES-D); and the Hamilton Rating Scale for Depression (HRSD); and/or
(f) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

25. The method of claim 14, wherein the ASD symptom to be evaluated is neurodegeneration correlated with ASD, and wherein:
(a) treating the neurodegeneration prevents and/or delays the onset and/or progression of the ASD; and/or
(b) the method results in treating, preventing, and/or delaying the progression and/or onset of neurodegeneration in the subject; and/or
(c) progression or onset of the neurodegeneration is slowed, halted, or reversed over a defined period of time following administration of the fixed escalated dose of the amino sterol or a salt or derivative thereof, as measured by a medically-recognized technique; and/or
(d) the neurodegeneration is positively impacted by the fixed escalated dose of the aminosterol or a salt or derivative thereof, as measured by a medically-recognized technique; and/or
(e) the positive impact and/or progression of neurodegeneration is measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or
(f) the progression or onset of neurodegeneration is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or
(g) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

26. The method of claim 7, wherein:
(a) the aminosterol or a salt or derivative thereof is administered in combination with at least one additional active agent to achieve either an additive or synergistic effect; and/or
(b) the additional active agent is administered via a method selected from the group consisting of concomitantly; as an admixture; separately and simultaneously or concurrently; and separately and sequentially; and/or
(c) the additional active agent is a different aminosterol from that administered in the method of claim 7;
(d) the method of claim 7 comprises a first aminosterol which is aminosterol 1436 or a salt or derivative thereof administered intranasally and a second amino sterol which is squalamine or a salt or derivative thereof administered orally; and/or
(e) the additional active agent is an active agent used to treat ASD or a symptom thereof; and/or
(f) the additional active agent is an active agent used to treat ASD or a symptom thereof wherein the additional active agent is selected from the group consisting of a serotonin-norepinephrine reuptake inhibitor such as venlafaxine, (Effexor®); selective serotonin reuptake inhibitor such as fluoxetine (Prozac®) or citalopram (Celexa®); N-methyl D-aspartate (NMDA) antagonists such as memantine (Namenda®); dopamine receptor antagonists such as haloperidol (Haldol®); a loop diuretic such as bumetanide; an acetylcholinesterase inhibitor such as rivastigmine (Exelon®); a central nervous system stimulant such as methylphenidate (Ritalin®) or amphetamine (Adderall®); and/or atypical antipsychotics such as risperidone (Risperdol®), aripiprazole (Abilify®), ziprasidone (Geodon®), paliperidone (Invega®), or clozapine (Clozaril®); and/or
(g) the aminosterol or a salt or derivative thereof is taken on an empty stomach, optionally within two hours of the subject waking; and/or
(h) no food is taken after about 60 to about 90 minutes of taking the aminosterol or a salt or derivative thereof; and/or
(i) the aminosterol or a salt or derivative thereof is a pharmaceutically acceptable grade of at least one aminosterol or a pharmaceutically acceptable salt or derivative thereof; and/or
(j) the aminosterol or a salt or derivative thereof is comprised in a composition further comprising one or more of the following: an aqueous carrier; a buffer; a sugar; and/or a polyol compound; and/or
(k) the subject is a human; and/or
(l) the subject is a member of a patient population or an individual at risk for developing ASD.

27. The method of claim 7, wherein the amino sterol or the salt or derivative thereof is:
(a) isolated from the liver of Squalus acanthias; and/or
(b) squalamine or a pharmaceutically acceptable salt thereof; and/or
(c) a squalamine isomer; and/or
(d) the phosphate salt of squalamine; and/or
(e) aminosterol 1436 or a pharmaceutically acceptable salt thereof; and/or
(f) an isomer of amino sterol 1436; and/or
(g) the phosphate salt of aminosterol 1436; and/or
(h) comprises a sterol nucleus and a polyamine attached at any position on the sterol, such that the molecule exhibits a net charge of at least +1; and/or
(i) comprises a bile acid nucleus and a polyamine, attached at any position on the bile acid, such that the molecule exhibits a net charge of at least +1; and/or
(j) a derivative modified to include one or more of the following:
(i) substitutions of the sulfate by a sulfonate, phosphate, carboxylate, or other anionic moiety chosen to circumvent metabolic removal of the sulfate moiety and oxidation of the cholesterol side chain;
(ii) replacement of a hydroxyl group by a non-metabolizable polar substituent, such as a fluorine atom, to prevent its metabolic oxidation or conjugation; and
(iii) substitution of one or more ring hydrogen atoms to prevent oxidative or reductive metabolism of the steroid ring system; and/or
(k) a derivative of squalamine modified through medicinal chemistry to improve bio-distribution, ease of administration, metabolic stability, or any combination thereof; and/or
(l) a synthetic amino sterol; and/or
(m) is selected from the group consisting of: 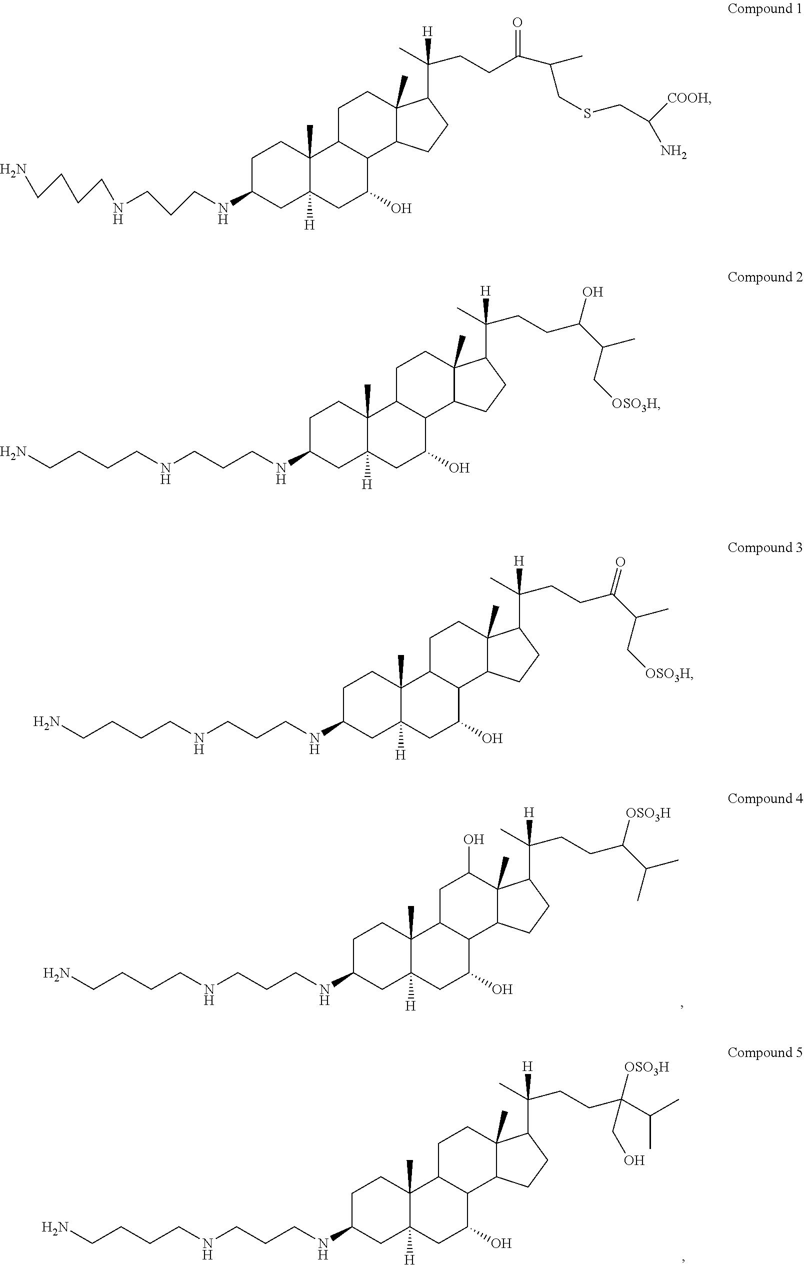 [chemical structure]
